SEQUENCE LISTING (88 sequences)

SEQ ID NO: 1 - protein, 8 aa, Artificial Sequence
  antibody fragment
GYTFTSYN

SEQ ID NO: 2 - protein, 8 aa, Artificial Sequence
  antibody fragment
IYPGNGDT

SEQ ID NO: 3 - protein, 11 aa, Artificial Sequence
  antibody fragment
ARYDYNYAMDY

SEQ ID NO: 4 - protein, 118 aa, Artificial Sequence
  antibody fragment
QAYLQQSGAELVRPGASVKMSCKASGYTFTSYNMHWVKQTPRQGLEWIGGIYPGNGDTSYNQKFKGKATLTVGKSSSTAYMQLSSLTSEDSAVYFCARYDYNYAMDYWGQGTSVTVSS

SEQ ID NO: 5 - protein, 330 aa, Artificial Sequence [sequence omitted]

SEQ ID NO: 6 - protein, 5 aa, Artificial Sequence
  antibody fragment
SSVSY

SEQ ID NO: 7 - protein, 3 aa, Artificial Sequence
  antibody fragment
ATS

SEQ ID NO: 8 - protein, 9 aa, Artificial Sequence
  antibody fragment
QQWTFNPPT

SEQ ID NO: 9 - protein, 106 aa, Artificial Sequence
  antibody fragment
QIVLSQSPAILSASPGEKVTMTCRASSSVSYMHWYQQKPGSSPKPWIYATSNLASGVPARFSGSGSGTSYSFTISRVEAEDAATYYCQQWTFNPPTFGGGTRLEIK

SEQ ID NO: 10 - protein, 106 aa, Artificial Sequence
  antibody fragment
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 9 aa, Artificial Sequence
  antibody fragment
GTFSRSAIS

SEQ ID NO: 12 - protein, 9 aa, Artificial Sequence
  antibody fragment
GTFSGYAIS

SEQ ID NO: 13 - protein, 9 aa, Artificial Sequence
  antibody fragment
GTFWRYAIS

SEQ ID NO: 14 - protein, 9 aa, Artificial Sequence
  antibody fragment
GTFGSYAIS

SEQ ID NO: 15 - protein, 9 aa, Artificial Sequence
  antibody fragment
GTFGTYAIS

SEQ ID NO: 16 - protein, 9 aa, Artificial Sequence
  antibody fragment
GTFSPKAIS

SEQ ID NO: 17 - protein, 9 aa, Artificial Sequence
  antibody fragment
YTLSSHGIT

SEQ ID NO: 18 - protein, 9 aa, Artificial Sequence
  antibody fragment
GTFSSYAIS

SEQ ID NO: 19 - protein, 17 aa, Artificial Sequence
  antibody fragment
VIIPAFGEANYAQKFQG

SEQ ID NO: 20 - protein, 17 aa, Artificial Sequence
  antibody fragment
VIIPAFGTANYAQKFQG

SEQ ID NO: 21 - protein, 17 aa, Artificial Sequence
  antibody fragment
VIIPIWGKANYAQKFQG

SEQ ID NO: 22 - protein, 17 aa, Artificial Sequence
  antibody fragment
GIYPAFGTANYAQKFQG

SEQ ID NO: 23 - protein, 17 aa, Artificial Sequence
  antibody fragment
GIYPRFGTANYAQKFQG

SEQ ID NO: 24 - protein, 17 aa, Artificial Sequence
  antibody fragment
VIIPIFGPANYAQKFQG

SEQ ID NO: 25 - protein, 17 aa, Artificial Sequence
  antibody fragment
WISAHSGHASNAQKVED

SEQ ID NO: 26 - protein, 17 aa, Artificial Sequence
  antibody fragment
GIIPIFGTANYAQKFQG

SEQ ID NO: 27 - protein, 13 aa, Artificial Sequence
  antibody fragment
ARGRQMFGAGIDF

SEQ ID NO: 28 - protein, 13 aa, Artificial Sequence
  antibody fragment
ARVWRALYHGMDV

SEQ ID NO: 29 - protein, 13 aa, Artificial Sequence
  antibody fragment
ARVHAALYHGMDV

SEQ ID NO: 30 - protein, 13 aa, Artificial Sequence
  antibody fragment
ARVHAALYYGMDV

SEQ ID NO: 31 - protein, 13 aa, Artificial Sequence
  antibody fragment
TRSSGSIDSNYVQ

SEQ ID NO: 32 - protein, 11 aa, Artificial Sequence
  antibody fragment
GGNNIGSKGVH

SEQ ID NO: 33 - protein, 7 aa, Artificial Sequence
  antibody fragment
EDNQRPS

SEQ ID NO: 34 - protein, 7 aa, Artificial Sequence
  antibody fragment
DDSDRPS

SEQ ID NO: 35 - protein, 11 aa, Artificial Sequence
  antibody fragment
QSYDSNNRHVI

SEQ ID NO: 36 - protein, 11 aa, Artificial Sequence
  antibody fragment
QVWDSSSDHWV

SEQ ID NO: 37 - protein, 26 aa, Artificial Sequence
  antibody fragment
EVQLVQSGAEVKKPGSSVKVSCKASG

SEQ ID NO: 38 - protein, 26 aa, Artificial Sequence
  antibody fragment
QVQLVQSGAEVKKPGSSVKVSCKASG

SEQ ID NO: 39 - protein, 14 aa, Artificial Sequence
  antibody fragment
WVRQAPGQGLEWMG

SEQ ID NO: 40 - protein, 30 aa, Artificial Sequence
  antibody fragment
RVTITADKSTSTAYMELSSLRSEDTAVYYC

SEQ ID NO: 41 - protein, 11 aa, Artificial Sequence
  antibody fragment
WGQGTLVTVSS

SEQ ID NO: 42 - protein, 22 aa, Artificial Sequence
  antibody fragment
NFMLTQPHSVSESPGKTVTISC

SEQ ID NO: 43 - protein, 15 aa, Artificial Sequence
  antibody fragment
WYQQRPGSAPTTVIY

SEQ ID NO: 44 - protein, 34 aa, Artificial Sequence
  antibody fragment
GVPDRFSGSIDSSSNSASLTISGLKTEDEADYYC

SEQ ID NO: 45 - protein, 10 aa, Artificial Sequence
  antibody fragment
FGGGTKLTVL

SEQ ID NO: 46 - protein, 120 aa, Artificial Sequence
  antibody fragment
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGRQMFGAGIDFWGQGTLVTVSS

SEQ ID NO: 47 - protein, 120 aa, Artificial Sequence
  antibody fragment
QVQLVQSGAEVKKPGSSVKVSCKASGGTFSRSAISWVRQAPGQGLEWMGVIIPAFGEANYAQKFQGRVTITADESTSTAYMELSSLRSEDTAVYYCARGRQMFGAGIDFWGQGTLVTVSS

SEQ ID NO: 48 - protein, 120 aa, Artificial Sequence
  antibody fragment
QVQLVQSGAEVKKPGSSVKVSCKASGGTFSGYAISWVRQAPGQGLEWMGVIIPAFGTANYAQKFQGRVTITADESTSTAYMELSSLRSEDTAVYYCARGRQMFGAGIDFWGQGTLVTVSS

SEQ ID NO: 49 - protein, 120 aa, Artificial Sequence
  antibody fragment
QVQLVQSGAEVKKPGSSVKVSCKASGGTFWRYAISWVRQAPGQGLEWMGVIIPIWGKANYAQKFQGRVTITADESTSTAYMELSSLRSEDTAVYYCARGRQMFGAGIDFWGQGTLVTVSS

SEQ ID NO: 50 - protein, 120 aa, Artificial Sequence
  antibody fragment
QVQLVQSGAEVKKPGSSVKVSCKASGGTFGSYAISWVRQAPGQGLEWMGGIYPAFGTANYAQKFQGRVTITADESTSTAYMELSSLRSEDTAVYYCARGRQMFGAGIDFWGQGTLVTVSS

SEQ ID NO: 51 - protein, 120 aa, Artificial Sequence
  antibody fragment
QVQLVQSGAEVKKPGSSVKVSCKASGGTFGTYAISWVRQAPGQGLEWMGGIYPRFGTANYAQKFQGRVTITADESTSTAYMELSSLRSEDTAVYYCARGRQMFGAGIDFWGQGTLVTVSS

SEQ ID NO: 52 - protein, 120 aa, Artificial Sequence
  antibody fragment
QVQLVQSGAEVKKPGSSVKVSCKASGGTFSPKAISWVRQAPGQGLEWMGVIIPIFGPANYAQKFQGRVTITADESTSTAYMELSSLRSEDTAVYYCARGRQMFGAGIDFWGQGTLVTVSS

SEQ ID NO: 53 - protein, 112 aa, Artificial Sequence
  antibody fragment
NFMLTQPHSVSESPGKTVTISCTRSSGSIDSNYVQWYQQRPGSAPTTVIYEDNQRPSGVPDRFSGSIDSSSNSASLTISGLKTEDEADYYCQSYDSNNRHVIFGGGTKLTVL

SEQ ID NO: 54 - protein, 26 aa, Artificial Sequence
  antibody fragment
EVQLVQSGGEVKKPGASVKVSCKASG

SEQ ID NO: 55 - protein, 30 aa, Artificial Sequence
  antibody fragment
RVTMTTDTSTNTAYMELRSLTADDTAVYYC

SEQ ID NO: 56 - protein, 22 aa, Artificial Sequence
  antibody fragment
LSVLTQPPSVSVAPGQTARITC

SEQ ID NO: 57 - protein, 15 aa, Artificial Sequence
  antibody fragment
WYQQKPGQAPVLVVY

SEQ ID NO: 58 - protein, 32 aa, Artificial Sequence
  antibody fragment
GIPERFSGSNSGNTATLTISRVEAGDEADYYC

SEQ ID NO: 59 - protein, 120 aa, Artificial Sequence
  antibody fragment
EVQLVQSGGEVKKPGASVKVSCKASGYTLSSHGITWVRQAPGQGLEWMGWISAHSGHASNAQKVEDRVTMTTDTSTNTAYMELRSLTADDTAVYYCARVWRALYHGMDVWGQGTLVTVSS

SEQ ID NO: 60 - protein, 120 aa, Artificial Sequence
  antibody fragment
EVQLVQSGGEVKKPGASVKVSCKASGYTLSSHGITWVRQAPGQGLEWMGWISAHSGHASNAQKVEDRVTMTTDTSTNTAYMELRSLTADDTAVYYCARVHAALYHGMDVWGQGTLVTVSS

SEQ ID NO: 61 - protein, 108 aa, Artificial Sequence
  antibody fragment
LSVLTQPPSVSVAPGQTARITCGGNNIGSKGVHWYQQKPGQAPVLVVYDDSDRPSGIPERFSGSNSGNTATLTISRVEAGDEADYYCQVWDSSSDHWVFGGGTKLTVL

SEQ ID NO: 62 - protein, 120 aa, Artificial Sequence
  antibody fragment
EVQLVQSGAEVKKPGSSVKVSCKASGGTFSRSAISWVRQAPGQGLEWMGVIIPAFGEANYAQKFQGRVTITADESTSTAYMELSSLRSEDTAVYYCARGRQMFGAGIDFWGQGTLVTVSS

SEQ ID NO: 63 - protein, 120 aa, Artificial Sequence
  antibody fragment
EVQLVQSGAEVKKPGSSVKVSCKASGGTFSGYAISWVRQAPGQGLEWMGVIIPAFGTANYAQKFQGRVTITADESTSTAYMELSSLRSEDTAVYYCARGRQMFGAGIDFWGQGTLVTVSS

SEQ ID NO: 64 - protein, 120 aa, Artificial Sequence
  antibody fragment
EVQLVQSGAEVKKPGSSVKVSCKASGGTFWRYAISWVRQAPGQGLEWMGVIIPIWGKANYAQKFQGRVTITADESTSTAYMELSSLRSEDTAVYYCARGRQMFGAGIDFWGQGTLVTVSS

SEQ ID NO: 65 - protein, 120 aa, Artificial Sequence
  antibody fragment
EVQLVQSGAEVKKPGSSVKVSCKASGGTFGSYAISWVRQAPGQGLEWMGGIYPAFGTANYAQKFQGRVTITADESTSTAYMELSSLRSEDTAVYYCARGRQMFGAGIDFWGQGTLVTVSS

SEQ ID NO: 66 - protein, 120 aa, Artificial Sequence
  antibody fragment
EVQLVQSGAEVKKPGSSVKVSCKASGGTFGTYAISWVRQAPGQGLEWMGGIYPRFGTANYAQKFQGRVTITADESTSTAYMELSSLRSEDTAVYYCARGRQMFGAGIDFWGQGTLVTVSS

SEQ ID NO: 67 - protein, 120 aa, Artificial Sequence
  antibody fragment
EVQLVQSGAEVKKPGSSVKVSCKASGGTFSPKAISWVRQAPGQGLEWMGVIIPIFGPANYAQKFQGRVTITADESTSTAYMELSSLRSEDTAVYYCARGRQMFGAGIDFWGQGTLVTVSS

SEQ ID NO: 68 - protein, 120 aa, Artificial Sequence
  antibody fragment
EVQLVQSGGEVKKPGASVKVSCKASGYTLSSHGITWVRQAPGQGLEWMGWISAHSGHASNAQKVEDRVTMTTDTSTNTAYMELRSLTADDTAVYYCARVHAALYYGMDVWGQGTLVTVSS

SEQ ID NO: 69 - DNA, 360 bp, Artificial Sequence
  antibody fragment
gaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacaagtccacgagcacagcctacatggagctgagcagcctgagatctgaggacacggcggtgtactactgcgctagaggcagacagatgttcggtgcaggcatcgatttctggggccagggcaccctggtcaccgtctcctca

SEQ ID NO: 70 - DNA, 360 bp, Artificial Sequence
  antibody fragment
caggtgcagctggtgcagtctggggctgaggtgaagaagcctgggtcctcggtgaaggtctcctgcaaggcttctggaggcaccttcagccgttcggctatcagctgggtgcgacaggcccctggacaagggcttgagtggatgggagttatcatccctgcgtttggtgaggcaaactacgcacagaagttccagggcagagtcacgattaccgcggacgaatccacgagcacagcctacatggagctgagcagcctgagatctgaggacacggcggtgtactactgcgctagaggcagacagatgttcggtgcaggcatcgatttctggggccagggcaccctggtcaccgtctcctca

SEQ ID NO: 71 - DNA, 360 bp, Artificial Sequence
  antibody fragment
caggtgcagctggtgcagtctggggctgaggtgaagaagcctgggtcctcggtgaaggtatcatgcaaggcttctggaggcaccttcagcgggtatgctatctcttgggtgcgacaggcccctggacaagggcttgagtggatgggagttatcatccctgcttttggtacagcaaactacgcacagaagttccagggcagagtcacgattaccgcggacgaatccacgagcacagcctacatggagctgagcagcctgagatctgaggacacggcggtgtactactgcgccagaggcagacagatgttcggtgcaggcatcgatttctggggccagggcaccctggtcaccgtctcctca

SEQ ID NO: 72 - DNA, 360 bp, Artificial Sequence
  antibody fragment
caggtgcagctggtgcagtctggggctgaggtgaagaagcctgggtcctcggtgaaggtctcctgcaaggcttctggaggcaccttctggaggtatgctatcagctgggtgcgacaggcccctggacaagggcttgagtggatgggagttatcatccctatctggggtaaagcaaactacgcacagaagttccagggcagagtcacgattaccgcggacgaatccacgagcacagcctacatggagctgagcagcctgagatctgaggacacggcggtgtactactgcgccagaggcagacagatgttcggtgcaggcatcgatttctggggccagggcaccctggtcaccgtctcctca

SEQ ID NO: 73 - DNA, 360 bp, Artificial Sequence
  antibody fragment
caggtgcagctggtgcagtctggggctgaggtgaagaagcctgggtcctcggtgaaggtctcctgcaaggcttctggaggcaccttcgggagctatgctatctcttgggtgcgacaggcccctggacaagggcttgagtggatgggagggatctatcctgcttttggtacagcaaactacgcacagaagttccagggcagagtcacgattaccgcggacgaatccacgagcacagcctacatggagctgagcagcctgagatctgaggacacggcggtgtactactgcgctagaggcagacagatgttcggtgcaggcatcgatttctggggccagggcaccctggtcaccgtctcctca

SEQ ID NO: 74 - DNA, 360 bp, Artificial Sequence
  antibody fragment
caggtgcagctggtgcagtctggggctgaggtgaagaagcctgggtcctcggtgaaggtctcctgcaaggcttctggaggcaccttcgggacgtatgctatcagctgggtgcgacaggcccctggacaagggcttgagtggatgggagggatctatcctaggtttggtacagcaaactacgcacagaagttccagggcagagtcacgattaccgcggacgaatccacgagcacagcctacatggagctgagcagcctgagatctgaggacacggcggtgtactactgcgctagaggcagacagatgttcggtgcaggcatcgatttctggggccagggcaccctggtcaccgtctcctca

SEQ ID NO: 75 - DNA, 360 bp, Artificial Sequence
  antibody fragment
caggtgcagctggtgcagtctggggctgaggtgaagaagcctgggtcctcggtgaaggtctcctgcaaggcttctggaggcaccttcagcccgaaggctatcagctgggtgcgacaggcccctggacaagggcttgagtggatgggagtgatcatccctatctttggtccggcaaactacgcacagaagttccagggcagagtcacgattaccgcggacgaatccacgagcacagcctacatggagctgagcagcctgagatctgaggacacggcggtgtactactgcgctagaggcagacagatgttcggtgcaggcatcgatttctggggccagggcaccctggtcaccgtctcctca

SEQ ID NO: 76 - DNA, 360 bp, Artificial Sequence
  antibody fragment
gaggttcagctggtgcagtctggaggtgaggtgaagaagcctggggcctcagtgaaggtctcctgcaaggcttctggttacacccttagcagccatggtatcacctgggtgcgacaggcccctggacaagggcttgagtggatgggatggatcagcgctcacagtggtcacgcaagcaatgcacagaaggtcgaggacagagtcaccatgaccacagacacatccacgaacacagcctacatggagctgaggagcctgacagctgacgacacggcggtgtactactgcgccagagtccatgccgccttgtactacggtatggacgtctgggggcaagggaccctggtcaccgtctcctca

SEQ ID NO: 77 - DNA, 360 bp, Artificial Sequence
  antibody fragment
gaggttcagctggtgcagtctggaggtgaggtgaagaagcctggggcctcagtgaaggtctcctgcaaggcttctggttacacccttagcagccatggtatcacctgggtgcgacaggcccctggacaagggcttgagtggatgggatggatcagcgctcacagtggtcacgcaagcaatgcacagaaggtcgaggacagagtcaccatgaccacagacacatccacgaacacagcctacatggagctgaggagcctgacagctgacgacacggcggtgtactactgcgccagagtccatgccgccttgtaccacggtatggacgtctgggggcaagggaccctggtcaccgtctcctca

SEQ ID NO: 78 - DNA, 360 bp, Artificial Sequence
  antibody fragment
gaggttcagctggtgcagtctggaggtgaggtgaagaagcctggggcctcagtgaaggtctcctgcaaggcttctggttacacccttagcagccatggtatcacctgggtgcgacaggcccctggacaagggcttgagtggatgggatggatcagcgctcacagtggtcacgcaagcaatgcacagaaggtcgaggacagagtcaccatgaccacagacacatccacgaacacagcctacatggagctgaggagcctgacagctgacgacacggcggtgtactactgcgccagagtgtggagggccttgtaccacggtatggacgtctgggggcaagggaccctggtcaccgtctcctca

SEQ ID NO: 79 - DNA, 336 bp, Artificial Sequence
  antibody fragment
aattttatgctgactcagccccactctgtgtcggagtctccggggaagacggtaaccatctcctgcacccgcagcagtggcagcattgacagcaactatgtgcagtggtaccagcagcgcccgggcagtgcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagcaacaataggcatgtgatattcggcggagggaccaagctgaccgtccta

SEQ ID NO: 80 - DNA, 324 bp, Artificial Sequence
  antibody fragment
ttgtctgtgctgactcagccaccctcagtgtcagtggccccaggacagacggccaggattacctgtgggggaaacaacattggaagtaaaggtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggacagtagtagtgatcattgggtgttcggcggagggaccaagctgaccgtccta

SEQ ID NO: 81 - protein, 120 aa, Artificial Sequence
  antibody fragment
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGRQMFGAGIDFWGQGTLVTVSS

SEQ ID NO: 82 - protein, 120 aa, Artificial Sequence; identical to SEQ ID NO: 68
  antibody fragment

SEQ ID NO: 83 - protein, 451 aa, Artificial Sequence
  antibody fragment
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 215 aa, Artificial Sequence
  antibody fragment
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 11 aa, Artificial Sequence
  antibody fragment
QSYDSNLRHVI

SEQ ID NO: 86 - protein, 11 aa, Artificial Sequence
  antibody fragment
QSYDSNIRHVI

SEQ ID NO: 87 - protein, 34 aa, Homo sapiens
GVPDRFSGSIDSSSNWASLTISGLKTEDEADYYC

SEQ ID NO: 88 - protein, 34 aa, Homo sapiens
GVPDRFSGSIDSSSNVASLTISGLKTEDEADYYC

(19) United States
(12) Patent Application Publication
(10) Pub. No.: US 2019/0247399 A1
(43) Pub. Date: Aug. 15, 2019

(54) COMBINATION OF AN ANTI-CD20 ANTIBODY, PI3 KINASE-DELTA INHIBITOR, AND ANTI-PD-1 OR ANTI-PD-L1 ANTIBODY FOR TREATING HEMATOLOGICAL CANCERS

(71) Applicants: TG Therapeutics, Inc., New York, NY (US); Rhizen Pharmaceuticals SA, La Chaux de Fonds (CH); Laboratoire Francais Du Fractionnement Et Des Biotechnologies, Les Ulis (FR)

(72) Inventors: Michael S. Weiss, New York, NY (US); Hari P. Miskin, New York, NY (US); Peter Sportelli, New York, NY (US)

(21) Appl. No.: 16/331,407

(22) PCT Filed: Sep. 8, 2017
(86) PCT No.: PCT/US2017/050825

§ 371 Date: Mar. 7, 2019

Related U.S. Application Data
(60) Provisional application No. 62/385,723, filed on Sep. 9, 2016.

(51) Int. Cl. A61K 31/519; A61P 35/02; C07K 16/28; A61K 9/00

(52) U.S. Cl. CPC A61K 31/519; A61P 35/02; A61K 9/0053; C07K 16/2818; A61K 9/0019; C07K 16/2827

(57) ABSTRACT

The present disclosure provides methods and kits for treating or slowing the progression of a hematological malignancy, by administering to a subject in need thereof a therapeutically effective amount of: (i) at least one inhibitor of PI3 kinase (PI3K)-delta (e.g., TGR-1202); (ii) at least one anti-CD20 antibody (e.g., ublituximab); and (iii) at least one anti-PD-1 antibody (e.g., pembrolizumab) or anti-PD-Ll antibody (e.g., atezolizumab). Treatment regimens are also provided.

### FIELD OF THE INVENTION

The present invention relates generally to the field of cancer therapy. More particularly, the present invention relates to methods and kits for treating or slowing the progression of hematological cancers, by administering to a subject in need thereof a therapeutically effective amount of: (i) at least one inhibitor of PI3 kinase (PI3K)-delta (e.g., TGR-1202); (ii) at least one anti-CD20 antibody (e.g., ublituximab); and (iii) at least one anti-PD1 antibody (e.g., pembrolizumab) or anti-PD-L1 antibody (e.g., atezolizumab).

### BACKGROUND OF THE INVENTION

Despite more than a century of scientific and clinical research, curing cancer remains a medical challenge. Cancer treatments have mainly relied on the combination of surgery, radiotherapy, and/or cytotoxic chemotherapies. Within the last decade, however, targeted cancer therapies have opened a new era in the field of oncology. Targeted cancer therapies are drugs designed to interfere with specific molecules necessary for tumor growth and progression; they are broadly classified into monoclonal antibodies (mAbs) or small molecules. Some examples of targeted therapies include monoclonal antibodies to CD20 (e.g., rituximab/Rituxan® for treating lymphomas), CD52 (e.g., alemtuzumab/Campath®), VEGF (e.g., bevacizumab/Avastin®), HER2 (e.g., trastuzumab/Herceptin® for treating Her2+ breast and stomach cancers), EGFR (e.g., cetuximab/Erbitux® for treating colorectal cancer), CTLA-4 (e.g., ipilimumab/Yervoy® for treating melanoma), and PD-1 (e.g., nivolumab/Opdivo® for treating squamous cell and non-squamous cell non-small cell lung cancer (NSCLC), and pembrolizumab/Keytruda® for treating NSCLC). Small molecule therapies target dysregulated pathways of cancer cells, e.g., RAS, RAF, PI3K, MEK, JAK, STAT, and BTK.

While effective hematological cancer therapies exist, suboptimal response, relapsed-refractory disease, and/or resistance to one or more therapeutic agents have remained a challenge. Further, patients with higher risk cytogenetic abnormalities still present with a less than optimal response to approved therapies and shorter duration of response and progression free survival. Accordingly, there is a need for more effective, safe, and durable targeted combination therapies for the treatment of hematological malignancies.

### BRIEF SUMMARY OF THE INVENTION

The present disclosure provides an innovative combination treatment and treatment regimen for patients with hematological malignancies.

In one aspect, the present disclosure provides a method for treating a subject afflicted with chronic lymphocytic leukemia (CLL) comprising administering to the subject in a treatment phase: (i) a therapeutically effective amount of a PI3-kinase delta inhibitor, wherein the PI3-kinase delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate, or prodrug thereof; (ii) a therapeutically effective amount of ublituximab; and (iii) a therapeutically effective amount of an anti-PD-1 antibody.

In one aspect, the present disclosure provides a PI3-kinase delta inhibitor and/or ublituximab for use in a method for treating a subject afflicted with chronic lymphocytic leukemia (CLL) comprising administering to the subject in a treatment phase: (i) a therapeutically effective amount of the PI3-kinase delta inhibitor, wherein the PI3-kinase delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate, or prodrug thereof; (ii) a therapeutically effective amount of ublituximab; and (iii) a therapeutically effective amount of an anti-PD-1 antibody.

In some embodiments, the anti-PD-1 antibody is pembrolizumab.

In some embodiments, the PI3-kinase delta inhibitor is in the form of a p-toluenesulfonic acid (PTSA) salt. In some embodiments, the PI3-kinase delta inhibitor is TGR-1202 (umbralisib tosylate).

In some embodiments, the PI3-kinase delta inhibitor, the ublituximab, and the pembrolizumab are administered to the subject simultaneously, sequentially, or both simultaneously and sequentially.

In some embodiments, the PI3-kinase delta inhibitor is administered daily at a dose from: about 200 to about 1200 mg, about 400 to about 1000 mg, about 400 to about 800 mg, about 600 mg, about 700 mg, about 800 mg, about 900 mg, about 1000 mg, or about 1200 mg.

In some embodiments, the PI3-kinase delta inhibitor is administered daily at a dose of about 800 mg. In some embodiments, the PI3-kinase delta inhibitor is micronized. In some embodiments, the PI3-kinase delta inhibitor is formulated for oral administration.

In some embodiments, ublituximab is administered in the treatment phase at a dose from: about 450 to about 1200 mg, about 450 to about 1000 mg, about 500 to about 1200 mg, about 500 to about 1000 mg, about 500 to about 900 mg, about 600 to about 1200 mg, about 600 to about 1000 mg, about 600 to about 900 mg, about 500 mg, about 600 mg, about 700 mg, about 750 mg, about 800 mg, about 900 mg, about 1000 mg, about 1100 mg, or about 1200 mg about once every 4 to 7 weeks, about once every 5 to 7 weeks, about once every 5 to 6 weeks, about once a week, about once every 2 weeks, about once every 3 weeks, about once every 4 weeks, about once every 5 weeks, about once every 6 weeks, or about once every 7 weeks.

In some embodiments, ublituximab is administered at a dose of about 900 mg about once every 6 weeks.

In some embodiments, the first dose of ublituximab is administered on day 1 of the sixth week after the treatment phase is initiated. In some embodiments, ublituximab is formulated for intravenous infusion.

In some embodiments, pembrolizumab is administered at a dose from: about 100 to about 300 mg, about 100 to about 200 mg, about 100 mg, about 150 mg, about 200 mg, or about 250 mg about once every 2 to 4 weeks, or about once every 3 to 4 weeks, or about once every 3 weeks. In some embodiments, pembrolizumab is administered at a dose of about 100 mg or 200 mg about once every 3 weeks. In some embodiments, the first dose of pembrolizumab is administered at a dose of about 100 mg. In some embodiments, pembrolizumab is formulated for intravenous infusion.

In some embodiments, the duration of the treatment phase is up to about 15 weeks, up to about 14 weeks, up to about 13 weeks, or up to about 12 weeks. In some embodiments, the duration of the treatment phase is about 12 weeks.

In some embodiments, the methods described herein further comprise, prior to the treatment phase, an induction phase, comprising administering to the subject: (i) a therapeutically effective amount of a PI3-kinase delta inhibitor, wherein the PI3-kinase delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate, or prodrug thereof; and (ii) a therapeutically effective amount of ublituximab. In some embodiments, the PI3-kinase delta inhibitor is in the form of a p-toluenesulfonic acid (PTSA) salt. In some embodiments, the PI3-kinase delta inhibitor is TGR-1202 (umbralisib tosylate).

In some embodiments, the PI3-kinase delta inhibitor and the ublituximab are administered to the subject simultaneously, sequentially or both simultaneously and sequentially during the induction phase.

In some embodiments, the PI3-kinase delta inhibitor is administered daily at a dose from: about 200 to about 1200 mg, about 400 to about 1000 mg, about 400 to about 800 mg, about 600 mg, about 700 mg, about 800 mg, about 900 mg, about 1000 mg, or about 1200 mg during the induction phase. In some embodiments, the PI3-kinase delta inhibitor is administered daily at a dose of about 800 mg during the induction phase. In some embodiments, the PI3-kinase delta inhibitor is micronized. In some embodiments, the PI3-kinase delta inhibitor is formulated for oral administration during the induction phase.

In some embodiments, ublituximab is administered at a dose from: about 450 to about 1200 mg, about 450 to about 1000 mg, about 600 to about 1200 mg, about 600 to about 1000 mg, about 600 to about 900 mg, about 600 mg, about 700 mg, about 800 mg, or about 900 mg about once every 1 to 3 weeks, about once every 1 to 2 weeks, about once every 1 week, or about once every 2 weeks during the induction phase. In some embodiments, ublituximab is administered at a dose of about 900 mg about once every 1 or 2 weeks during the induction phase. In some embodiments, ublituximab is formulated for intravenous infusion during the induction phase.

In some embodiments, the first dose of ublituximab is administered on day 1 of the induction phase. In some embodiments, the first dose of ublituximab, during the induction phase, is divided into 2 or 3 sub-doses to be administered in 2 or 3 consecutive days during the induction phase, or is divided into 2 sub-doses to be administered in 2 consecutive days.

In some embodiments, the first sub-dose of ublituximab comprises up to 150 mg of ublituximab. In some embodiments, the second sub-dose of ublituximab comprises up to 750 mg of ublituximab.

In some embodiments, the duration of the induction phase is up to about 12 weeks, up to about 11 weeks, up to about 10 weeks, up to about 9 weeks, or up to about 8 weeks. In some embodiments, the duration of the induction phase is about 8 weeks.

In some embodiments, the methods described herein further comprise, after the treatment phase, a maintenance phase, which comprises administering to the subject a therapeutically effective amount of a P13-kinase delta inhibitor, or a pharmaceutically acceptable salt, solvate or prodrug thereof. In some embodiments, the PI3-kinase delta inhibitor is administered daily at a dose from: about 200 to about 1200 mg, about 400 to about 1000 mg, about 400 to about 800 mg, about 600 mg, about 700 mg, about 800 mg, about 900 mg, about 1000 mg, or about 1200 mg during the maintenance phase. In some embodiments, the PI3-kinase delta inhibitor is administered daily at a dose of about 800 mg during the maintenance phase. In some embodiments, the PI3-kinase delta inhibitor is micronized. In some embodiments, the PI3-kinase delta inhibitor is formulated for oral administration during the maintenance phase.

In some embodiments, the duration of the maintenance phase is as long as clinical benefit is observed, or until unmanageable toxicity or disease progression occurs. In some embodiments, the maintenance phase ends when disease progression occurs. In some embodiments, the duration of the maintenance phase is at least 3 weeks.

In some embodiments, the subjects that are treated with the methods described herein are afflicted with relapsed-refractory CLL.

In one aspect, the present disclosure provides a method for treating a subject afflicted with relapsed-refractory chronic lymphocytic leukemia (CLL) comprising administering to the subject during a treatment phase: (i) a daily amount of about 800 mg of a PI3-kinase delta inhibitor, wherein the PI3-kinase delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate or prodrug thereof; (ii) about 900 mg of ublituximab once every 6 weeks, wherein the first dose of ublituximab is administered on day 1 of the sixth week after the treatment phase is initiated; and (iii) about 100 mg or 200 mg of pembrolizumab once every 3 weeks, wherein the first dose of pembrolizumab is administered on day 1 when the treatment phase is initiated; wherein the duration of the treatment phase is about 12 weeks; and wherein the PI3-kinase delta inhibitor, the ublituximab, and the pembrolizumab are administered to the subject simultaneously, sequentially, or both simultaneously and sequentially.

In one aspect, the present disclosure provides a PI3-kinase delta inhibitor and/or ublituximab for use in a method for treating a subject afflicted with relapsed-refractory chronic lymphocytic leukemia (CLL) comprising administering to the subject during a treatment phase: (i) a daily amount of about 800 mg of the PI3-kinase delta inhibitor, wherein the PI3-kinase delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate or prodrug thereof; (ii) about 900 mg of ublituximab once every 6 weeks, wherein the first dose of ublituximab is administered on day 1 of the sixth week after the treatment phase is initiated; and (iii) about 100 mg or 200 mg of pembrolizumab once every 3 weeks, wherein the first dose of pembrolizumab is administered on day 1 when the treatment phase is initiated; wherein the duration of the treatment phase is about 12 weeks; and wherein the PI3-kinase delta inhibitor, the ublituximab, and the pembrolizumab are administered to the subject simultaneously, sequentially, or both simultaneously and sequentially.

In some embodiments, the methods described herein further comprise, prior to the treatment phase, an induction phase, comprising administering to the subject: (i) a daily amount of about 800 mg of a PI3-kinase delta inhibitor, wherein the PI3-kinase delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate or prodrug thereof daily; and (ii) about 900 mg of ublituximab once every 1 or 2 weeks; wherein the first dose of ublituximab is administered on day 1 of the induction phase; wherein the duration of the induction phase is about 8 weeks; and wherein the PI3-kinase delta inhibitor and the ublituximab are administered to the subject simultaneously, sequentially, or both simultaneously and sequentially.

In some embodiments, the first dose of ublituximab is divided into 2 sub-doses during the induction phase, wherein the first sub-dose comprises up to 150 mg of ublituximab; and the second sub-dose comprises up to 750 mg of ublituximab; and wherein the first and second sub-doses are administered on day 1 and day 2 of the induction phase, respectively.

In some embodiments, the methods of the invention further comprise, after the treatment phase, a maintenance phase, comprising administering to the subject daily about 800 mg of a PI3-kinase delta inhibitor, or a pharmaceutically acceptable salt, solvate or prodrug thereof, wherein the duration of the maintenance phase is at least 3 weeks. In some embodiments, the PI3-kinase delta inhibitor is micronized and is formulated for oral administration. In some embodiments, the ublituximab and the pembrolizumab are formulated for intravenous infusion.

In one aspect, the present disclosure provides a kit for treating a subject afflicted with relapsed-refractory CLL, the kit comprising: (i) a single dose or multiple doses of ublituximab; (ii) a single dose or multiple doses of a PI3-kinase delta inhibitor, wherein the PI3-kinase delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate, or prodrug thereof; (iii) a single dose or multiple doses of an anti-PD-1 antibody; and (iv) instructions for using said ublituximab, said PI3-kinase delta inhibitor, and said anti-PD-1 antibody according to the methods described herein. In some embodiments, the anti-PD-1 antibody is pembrolizumab.

In one aspect, the present disclosure provides a method of treating a subject afflicted with a hematologic cancer, comprising administering to the subject in a treatment phase: (i) a therapeutically effective amount of a PI3 kinase-delta inhibitor; (ii) a therapeutically effective amount of an anti-CD20 antibody; and (iii) a therapeutically effective amount of an anti-PD-1 or anti-PD-L1 antibody.

In one aspect, the present disclosure provides a PI3-kinase delta inhibitor and/or ublituximab for use in a method of treating a subject afflicted with a hematologic cancer, comprising administering to the subject in a treatment phase: (i) a therapeutically effective amount of the PI3 kinase-delta inhibitor; (ii) a therapeutically effective amount of an anti-CD20 antibody; and (iii) a therapeutically effective amount of an anti-PD-1 or anti-PD-L1 antibody.

In some embodiments, the PI3 kinase-delta inhibitor is (S)-2-(1-(4-amino-3-(3-fluoro-4-isopropoxyphenyl)-1H-pyrazolo [3,4-d]pyrim-idin-1-yl)ethyl-6-fluoro-3-(3-fluorophenyl)-4H-chromen-4-one, or a pharmaceutically acceptable salt, solvate, or prodrug thereof. In some embodiments, the PI3 kinase-delta inhibitor is in the form of a p-toluenesulfonic acid (PTSA) salt. In some embodiments, the PI3 kinase-delta inhibitor is TGR-1202 (umbralisib tosylate).

In some embodiments, the anti-CD20 antibody is ublituximab or an antibody fragment that binds the same epitope as ublituximab.

In some embodiments, the anti-PD-1 antibody is nivolumab, pembrolizumab, or pidilizumab.

In some embodiments, the anti-PD-L1 antibody is CTI-07, CTI-09, CTI-48, CTI-49, CTI-50, CTI-76, CTI-77, CTI-78, CTI-57, CTI-58, CTI-97, CTI-98, CTI-92, CTI-95, CTI-93, CTI-94, CTI-96, durvalumab, BMS-936559, atezolizumab, or avelumab.

In some embodiments, the anti-PD-L1 antibody is CTI-48.

In some embodiments, the hematological cancer is lymphoma, leukemia, or myeloma.

In some embodiments, the hematological cancer is acute lymphocytic leukemia (ALL), acute myeloid leukemia (AML), chronic lymphocytic leukemia (CLL), small lymphocytic lymphoma (SLL), multiple myeloma (MM), non-Hodgkin's lymphoma (NHL), mantle cell lymphoma (MCL), follicular lymphoma (FL), Waldenstrom's macroglobulinemia (WM), diffuse large B-cell lymphoma (DLBCL), marginal zone lymphoma (MZL), hairy cell leukemia (HCL), Burkitt's lymphoma (BL), or Richter's transformation.

In some embodiments, the hematological cancer expresses PD-1 or PD-L1.

In some embodiments, the hematological cancer is relapsed-refractory disease. In some embodiments, the hematological cancer is relapsed-refractory CLL.

In one aspect, the present disclosure provides a method of treating a subject afflicted with a hematologic cancer, comprising administering to the subject in a treatment phase: (i) a therapeutically effective amount of a PI3-kinase delta inhibitor, wherein the PI3-kinase delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate, or prodrug thereof; (ii) a therapeutically effective amount of ublituximab; and (iii) a therapeutically effective amount of an anti-PD-1 antibody or anti-PD-L1 antibody.

In one aspect, the present disclosure provides a PI3-kinase delta inhibitor and/or ublituximab for use in a method of treating a subject afflicted with a hematologic cancer, comprising administering to the subject in a treatment phase: (i) a therapeutically effective amount of the PI3-kinase delta inhibitor, wherein the PI3-kinase delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate, or prodrug thereof; (ii) a therapeutically effective amount of ublituximab; and (iii) a therapeutically effective amount of an anti-PD-1 antibody or anti-PD-L1 antibody.

In some embodiments, the anti-PD-1 antibody is nivolumab, pembrolizumab, or pidilizumab.

In some embodiments, the anti-PD-L1 antibody is CTI-07, CTI-09, CTI-48, CTI-49, CTI-50, CTI-76, CTI-77, CTI-78, CTI-57, CTI-58, CTI-97, CTI-98, CTI-92, CTI-95, CTI-93, CTI-94, CTI-96, durvalumab, BMS-936559, atezolizumab, or avelumab.

In some embodiments, the anti-PD-L1 antibody is CTI-48.

In some embodiments, the subject treated for a hematological cancer with the methods described herein is a human.

In some embodiments, the method described herein further comprises, prior to the treatment phase, an induction phase, comprising administering to the subject: (i) a therapeutically effective amount of a PI3 kinase-delta inhibitor, wherein the PI3-kinase delta inhibitor is (S)-2-(1-(4-amino-3-(3-fluoro-4-isopropoxyphenyl)-1H-pyrazolo [3,4-d]pyrim-idin-1-yl)ethyl)-6-fluoro-3-(3-fluorophenyl)-4H-chromen-4-one, or a pharmaceutically acceptable salt, solvate, or prodrug thereof; and (ii) a therapeutically effective amount of ublituximab. In some embodiments, the PI3 kinase-delta inhibitor is in the form of a p-toluenesulfonic acid (PTSA) salt. In some embodiments, the PI3 kinase-delta inhibitor is TGR-1202 (umbralisib tosylate).

In some embodiments, the method described herein further comprises, after the treatment phase, a maintenance phase, comprising administering to the subject a therapeutically effective amount of a PI3 kinase-delta inhibitor, or a pharmaceutically acceptable salt, solvate or prodrug thereof.

In some embodiments, the PI3 kinase-delta inhibitor, the ublituximab, and the anti-PD-1 antibody or anti-PD-L1 antibody are administered to the subject simultaneously, sequentially, or both simultaneously and sequentially.

In one aspect, the present disclosure provides a kit for treating a subject afflicted with a hematological cancer, the kit comprising: (i) a single dose or multiple doses of ublituximab; (ii) a single dose or multiple doses of a PI3 kinase-delta inhibitor, wherein the PI3 kinase-delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate, or prodrug thereof; (iii) a single dose or multiple doses of an anti-PD-1 or anti-PD-L1 antibody; and (iv) instructions for using said ublituximab, said PI3 kinase-delta inhibitor, and said anti-PD-1 or anti-PD-L1 antibody, according to the methods of the present invention.

In some embodiments, the anti-PD-1 antibody in the kit is pembrolizumab. In some embodiments, the anti-PD-L1 antibody in the kit is atezolizumab. In some embodiments, the PI3 kinase-delta inhibitor in the kit is in the form of a p-toluenesulfonic acid (PTSA) salt. In some embodiments, the PI3 kinase-delta inhibitor in the kit is TGR-1202 (umbralisib tosylate).

### BRIEF DESCRIPTION OF THE DRAWINGS

FIG. 1 is a graph showing the percent inhibition of PD-1 binding to PD-L-1+cells by anti-PD-L1 antibodies, CTI-09, CTI-48, CTI-50, CTI-58, and a clinical control. The results were obtained using FACS analysis.

FIGS. 2A-2C are graphs showing the binding kinetics of exemplary anti-PD-L1 antibody CTI-48 against human PD-L1 (FIG. 2A), mouse PD-L1 (FIG. 2B), and cyno PD-L1 (FIG. 2C).

FIG. 3 is a bar graph showing that exemplary anti-PD-L1 antibody CTI-48 exhibits ADCC activity on PD-L1+ lymphoma cells with primary NK cells.

FIG. 4 is a graph showing the reversal of T-cell inhibition with PD-L1 in a reporter (NFAT) bioassay of immunoblockade with select anti-PD-L1 antibodies, CTI-48 and CTI-49, and a clinical control mAb.

FIG. 5 is a graph showing blocking of PD-L1 binding to B7.1 by the PD-L1 antibody, CTI-48, and a clinical control mAb.

FIG. 6 is a bar graph showing the effect of the disclosed PD-L1 antibodies on IFN-γ production. Antibodies were dosed into mixed lymphocyte reaction (MLR) cultures at a concentration of 10 μg/mL. Data were normalized to vehicle control and are presented as combined mean+SEM (n=6). *p<0.05**p<0.01,***p<0.001 indicates statistical significance when compared to appropriate isotype control (hIgG1) using Ordinary one-way ANOVA with Dunnett's multiple comparison post-hoc test. This figure shows a side-by-side comparison of PD-L1 antibody, CTI-48, and a clinical control mAb.

FIG. 7 is a Kaplan-Meier plot of months of progression-free survival in 9 patients with CLL according to the phase 1/2 study discussed in Example 1.

FIG. 8 is a graphic parallel representation (a.k.a. “swimmers plot”) of each of the 9 patients with CLL studied in Example 1, and their number of days of progression-free survival during each phase (induction/consolidation/maintenance) of the clinical study. Each bar represents each patient in the study.

### DETAILED DESCRIPTION OF THE INVENTION

I. Definitions

To facilitate an understanding of the present invention, a number of terms and phrases are defined below.

Open terms such as “include,” “including,” “contain,” “containing” and the like mean “comprising.” These open-ended transitional phrases are used to introduce an open ended list of elements, method steps, or the like that does not exclude additional, unrecited elements or method steps.

The term “CD20” (also known as B lymphocyte CD20 antigen, MS4A1, B lymphocyte surface antigen B1, Bp35, Leukocyte surface antigen Leu-16) refers to any native CD20, unless otherwise indicated. The term “CD20” encompasses “full-length,” unprocessed CD20 as well as any form of CD20 that results from processing within the cell. The term also encompasses naturally occurring variants of CD20, e.g., splice variants, allelic variants and isoforms. The CD20 polypeptides described herein can be isolated from a variety of sources, such as from human tissue types or from another source, or prepared by recombinant or synthetic methods. Examples of CD20 sequences include, but are not limited to, NCBI reference numbers NP_068769.2 and NP_690605.1.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

A “blocking” antibody or an “antagonist” antibody is one which inhibits or reduces biological activity of the antigen it binds, such as CD20. In a certain embodiment, blocking antibodies or antagonist antibodies substantially or completely inhibit the biological activity of the antigen. Desirably, the biological activity is reduced by 10%, 20%, 30%, 50%, 70%, 80%, 90%, 95%, or even 100%.

The term “anti-CD20 antibody” or “an antibody that binds to CD20” refers to an antibody that is capable of binding CD20 with sufficient affinity such that the antibody is useful as a diagnostic and/or therapeutic agent in targeting CD20. The extent of binding of an anti-CD20 antibody to an unrelated, non-CD20 protein is less than about 10% of the binding of the antibody to CD20 as measured, e.g., by a radioimmunoassay (RIA). In certain embodiments, an antibody that binds to CD20 has a dissociation constant (Kd) of ≤1 μM, ≤100 nM, ≤10 nM, ≤1 nM, or ≤0.1 nM.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, single chain antibodies, and multispecific antibodies formed from antibody fragments.

A “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

The term “humanized antibody” refers to forms of non-human (e.g., murine) antibodies that are specific immunoglobulin chains, chimeric immunoglobulins, or fragments thereof that contain minimal non-human (e.g., murine) sequences. Typically, humanized antibodies are human immunoglobulins in which residues from the complementary determining region (CDR) are replaced by residues from the CDR of a non-human species (e.g., mouse, rat, rabbit, hamster) that have the desired specificity, affinity, and capability (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody from a non-human species that has the desired specificity, affinity, and capability. The humanized antibody can be further modified by the substitution of additional residues either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in U.S. Pat. Nos. 5,225,539 or 5,639,641.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs), which are also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al., Sequences of Proteins of Immunological Interest, 5^{th} ed., National Institutes of Health, Bethesda, Md. (1991)); and (2) an approach based on crystallographic studies of antigen-antibody complexes (Al-lazikani et al, J. Molec. Biol. 273:927-948 (1997)). In addition, combinations of these two approaches are sometimes used in the art to determine CDRs.

The Kabat numbering system is generally used when referring to a residue in the variable domain (approximately residues 1-107 of the light chain and residues 1-113 of the heavy chain) (e.g., Kabat et al., supra).

The amino acid position numbering as in Kabat, refers to the numbering system used for heavy chain variable domains or light chain variable domains of the compilation of antibodies in Kabat et al., supra. Using this numbering system, the actual linear amino acid sequence can contain fewer or additional amino acids corresponding to a shortening of, or insertion into, a FR or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The term “human antibody” means an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described herein.

“Programmed Death-1” or “PD-1” refers to a cell surface immunoinhibitory receptor belonging to the CD28 family of T-cell regulators. PD-1 is expressed, upon activation, in B cells, T cells, monocytes, and natural killer cells (NKT). PD-1 binds to two ligands, PD-L1 and PD-L2. The term “PD-1,” as used herein, includes human PD-1 (hPD-1), variants, isoforms, and species homologs of hPD-1, and analogs having at least one common epitope with hPD-1. The complete hPD-1 sequence can be found under GenBank Accession No. U64863.

“Programmed Death Ligand-1” or “PD-L1” (also known as B7-H1) is one of two cell surface glycoprotein ligands for PD-1 (the other being PD-L2, also known as B7-DC) that down-regulate T cell activation and cytokine secretion upon binding to PD-1. The term “PD-L1,” as used herein, includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

The phrase “substantially similar,” or “substantially the same,” as used herein, denotes a sufficiently high degree of similarity between two numeric values (generally one associated with an antibody of the invention and the other associated with a reference/comparator antibody) such that one of skill in the art would consider the difference between the two values to be of little or no biological and/or statistical significance within the context of the biological characteristics measured by said values (e.g., Kd values). The difference between said two values is less than about 50%, less than about 40%, less than about 30%, less than about 20%, or less than about 10% as a function of the value for the reference/comparator antibody.

A polypeptide, antibody, polynucleotide, vector, cell, or composition that is “isolated” is in a form not found in nature. Isolated polypeptides, antibodies, polynucleotides, vectors, cells or compositions include those which have been purified to a degree that they are no longer in a form in which they are found in nature. In some embodiments, an antibody, polynucleotide, vector, cell, or composition that is isolated is substantially pure.

As used herein, “substantially pure” refers to material which is at least 50% pure (i.e., free from contaminants), at least 90% pure, at least 95% pure, at least 98% pure, or at least 99% pure.

“Polynucleotide” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide can comprise modified nucleotides, such as methylated nucleotides and their analogs. If present, modification to the nucleotide structure can be imparted before or after assembly of the polymer. The sequence of nucleotides can be interrupted by non-nucleotide components.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain embodiments, the polypeptides can occur as single chains or associated chains.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. One such non-limiting example of a sequence alignment algorithm is the algorithm described in Karlin et al, Proc. Natl. Acad. Sci. 87:2264-2268 (1990), as modified in Karlin et al., Proc. Natl. Acad. Sci. 90:5873-5877 (1993), and incorporated into the NBLAST and XBLAST programs (Altschul et al., Nucleic Acids Res. 25:3389-3402 (1991)). In certain embodiments, Gapped BLAST can be used as described in Altschul et al., Nucleic Acids Res. 25:3389-3402 (1997). BLAST-2, WU-BLAST-2 (Altschul et al., Methods in Enzymology 266:460-480 (1996)), ALIGN, ALIGN-2 (Genentech, South San Francisco, Calif.) or Megalign (DNASTAR) are additional publicly available software programs that can be used to align sequences. In certain embodiments, the percent identity between two nucleotide sequences is determined using the GAP program in GCG software (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch (J. Mol. Biol. 48:444-453 (1970)), can be used to determine the percent identity between two amino acid sequences (e.g., using either a Blossum 62 matrix or a PAM250 matrix, and a gap weight of 16, 14, 12, 10, 8, 6, or 4 and a length weight of 1, 2, 3, 4, 5). Alternatively, in certain embodiments, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. Appropriate parameters for maximal alignment by particular alignment software can be determined by one skilled in the art. In certain embodiments, the default parameters of the alignment software are used. In certain embodiments, the percentage identity “X” of a first amino acid sequence to a second sequence amino acid is calculated as 100 x (Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be longer than the percent identity of the second sequence to the first sequence.

As a non-limiting example, whether any particular polynucleotide has a certain percentage sequence identity (e.g., is at least 80% identical, at least 85% identical, at least 90% identical, and in some embodiments, at least 95%, 96%, 97%, 98%, or 99% identical) to a reference sequence can, in certain embodiments, be determined using the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2: 482 489 (1981), to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed.

In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In certain embodiments, identity exists over a region of the sequences that is at least about 10, about 20, about 40-60 residues in length or any integral value therebetween, or over a longer region than 60-80 residues, at least about 90-100 residues, or the sequences are substantially identical over the full length of the sequences being compared, such as the coding region of a nucleotide sequence for example.

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

The terms “cancer” and “cancerous” refer to or describe the physiological condition in mammals in which a population of cells are characterized by uncontrolled or unregulated cell growth. Examples of cancer include, e.g., carcinoma, lymphoma, blastoma, sarcoma, and leukemia.

As used herein, the term “hematological cancer” or “hematological malignancy” refers to any type of cancer (as defined above) that affects blood cells (e.g., T or B cells), bone marrow, or lymph nodes. One skilled in the art would understand that the three major categories of hematological cancers are lymphomas, leukemias, and myelomas. The malignancy may be indolent or aggressive. Non-limiting examples of hematological cancers that may be treated with the methods or kits of the present invention include acute lymphocytic leukemia (ALL), acute myeloid leukemia (AML), chronic lymphocytic leukemia (CLL), small lymphocytic lymphoma (SLL), multiple myeloma (MM), non-Hodgkin's lymphoma (NHL), mantle cell lymphoma (MCL), follicular lymphoma (FL), Waldenstrom's macroglobulinemia (WM), diffuse large B-cell lymphoma (DLBCL), marginal zone lymphoma (MZL), which includes extranodal MZL, nodal MZL, and splenic MZL, hairy cell leukemia (HCL), Burkitt's lymphoma (BL), and Richter's transformation. In some embodiments, the DLBCL is an activated B-cell DLBCL (ABC-DLBCL), a germinal center B-cell like DLBCL (GBC-DLBCL), a double hit DLBCL (DH-DLBCL), or a triple hit DLBCL (TH-DLBCL). In some embodiments, certain CLLs (or other leukemias, such as the ones described herein) are considered “high risk” due to the presence of one of more genetic mutations. As used herein, “high risk” CLL, for example, means CLL characterized by at least one of the following genetic mutations: 17p del; llq del; p53; unmutated IgVH together with ZAP-70+ and/or CD38+; and trisomy 12.

“Tumor” and “neoplasm” refer to any mass of tissue that results from excessive cell growth or proliferation, either benign (noncancerous) or malignant (cancerous) including pre-cancerous lesions.

The terms “cancer cell,” “tumor cell,” and grammatical equivalents refer to the total population of cells derived from a tumor or a pre-cancerous lesion, including both non-tumorigenic cells, which comprise the bulk of the tumor cell population, and tumorigenic stem cells (cancer stem cells). As used herein, the term “tumor cell” will be modified by the term “non-tumorigenic” when referring solely to those tumor cells lacking the capacity to renew and differentiate to distinguish those tumor cells from cancer stem cells.

The term “relapsed” cancer in a patient refers to patients who have previously achieved either a complete or partial remission, but after a period of 6 or more months, demonstrate evidence of disease progression.

The term “refractory” cancer in a patient refers to patients who have experienced treatment failure or disease progression within six months from the last anti-cancer therapy.

The term “relapsed-refractory CLL,” or “r/r CLL,” refers to CLL that occurs in patients who have previously achieved either a complete or partial remission by the International Workshop on CLL (IWCLL) response criteria (Hallek, M. et al., “Guidelines for the diagnosis and treatment of chronic lymphocytic leukemia: a report from the International Workshop on Chronic Lymphocytic Leukemia updating the National Cancer Institute—Working Group 1996 guidelines,” Blood 111:5446-5456 (2008), erratum in Blood 112: 5259 (2008)), but then develop progressive disease after a period of six months or more.

A tumor which “does not respond” or “responds poorly” to treatment (with, for example, an anti-CD20 antibody) does not show statistically significant improvement in response to that treatment when compared to no treatment or treatment with a placebo in a recognized animal model or human clinical trial, or which responds to an initial treatment, but grows as treatment continues.

The term “pharmaceutical formulation” refers to a preparation that is in such a form as to permit the biological activity of the active ingredient to be effective, and which contains no additional components which are unacceptably toxic to a subject to which the formulation would be administered. Such formulations can be sterile.

The term “therapeutically effective amount” refers to the amount of a therapeutic agent (e.g., an antibody or a small molecule) that is effective to “treat” a disease or disorder in a subject or mammal. In the case of cancer, the therapeutically effective amount of the agent can reduce the number of cancer cells, reduce the tumor size, inhibit (i.e., slow to some extent or stop) cancer cell infiltration into peripheral organs, inhibit (i.e., slow to some extent or stop) tumor metastasis, inhibit (to some extent) tumor growth, and/or relieve (to some extent) one or more of the symptoms associated with the cancer. See the definition herein of “treating.” To the extent the drug can prevent growth and/or kill existing cancer cells, it can be cytostatic and/or cytotoxic. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount may be less than the therapeutically effective amount.

Terms such as “treating,” “treatment,” “to treat,” “having a therapeutic effect,” alleviating,” “to alleviate,” or “slowing the progression of” refer to both 1) therapeutic measures that cure, slow down, lessen symptoms of, and/or halt progression of a diagnosed pathologic condition or disorder, such as a hematological malignancy, and 2) prophylactic or preventative measures that prevent and/or slow the development of a targeted pathologic condition or disorder. Thus, those in need of treatment include those already with the disorder; those prone to have the disorder; and those in whom the disorder is to be prevented. In certain embodiments, a subject is successfully “treated” for cancer according to the methods of the present invention if the patient shows one or more of the following: reduction in cachexia, increase in survival time, elongation in time to tumor progression, reduction in tumor mass, reduction in tumor burden and/or a prolongation in time to tumor metastasis, time to tumor recurrence or progressive disease, tumor response, complete response (CR), partial response (PR), stable disease, progression free survival (PFS), overall survival (OS), each as measured by standards set by the National Cancer Institute (NCI) and the U.S. Food and Drug Administration (FDA) for the approval of new drugs. See Johnson et al, J. Clin. Oncol. 21:1404-1411 (2003). In some embodiments, the “therapeutic effect,” as defined above, also encompasses a reduction in toxicity or adverse side effects, and/or an improvement in tolerability.

“Administering” refers to the physical introduction of a composition comprising a therapeutic agent to a subject, using any of the various methods and delivery systems known to those skilled in the art. Routes of administration include oral, mucosal, topical, intravenous, intramuscular, subcutaneous, intraperitoneal, spinal, or other parenteral routes of administration, for example, by injection or infusion. The phrase “parenteral administration” as used herein means modes of administration includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Administering can be performed, for example, once, a plurality of times, and/or over one or more extended periods.

A “combination” of (1) an anti-CD20 antibody (e.g., ublituximab); (2) a PI3K-delta selective inhibitor (e.g., TGR-1202); and (3) an anti-PD-1 antibody (e.g., pembrolizumab) or anti-PD-L1 antibody (e.g., atezolizumab), refers to the administration of one or more of each of these agents to the same subject simultaneously, sequentially, or both simultaneously and sequentially. Generally, unless inferred differently from context, a “combination of agents” refers to the administration of one or more of each of these three agents to the same subject simultaneously, sequentially, or both simultaneously and sequentially.

By way of example, administration of an anti-CD20 antibody preceding or following (e.g., by hour(s), day(s), week(s), or month(s)) administration of a PI3K-delta selective inhibitor, preceding or following (e.g., by hour(s), day(s), week(s), or month(s)) administration of an anti-PD-1 or anti-PD-L1 antibody, constitutes administration of a combination of agents, regardless of whether the agents are administered together in a single pharmaceutical formulation or are administered in separate pharmaceutical formulations by either the same or different routes of administration. As will be apparent to one skilled in the art from the context, a “combination of agents” can further include administration of one or more additional therapeutic agents, as described herein.

An “induction phase” or “induction therapy,” as used herein, refers to the administration of a first agent, or combination of agents, prior to a treatment phase, as described herein. If the treatment in the induction phase does not result in a complete response or it causes severe side effects, a treatment phase may be initiated, where other agents may be added or used instead (see “treatment phase”). Induction is also called primary therapy, or primary treatment, and is administered with the goal of inducing some initial reduction in disease burden. For example, induction therapy can include administration of an anti-CD20 antibody (e.g., ublituximab) and a PI3K-delta inhibitor (e.g., TGR-1202). In some embodiments, however, induction therapy is not administered as part of a subject's treatment regimen.

A “treatment phase,” as used herein, generally refers to the treatment that is administered to a subject following induction therapy. In some embodiments, the treatment phase is used to kill any remaining malignant hematologic cells following induction therapy. For example, if an anti-CD20 antibody and a PI3K-delta inhibitor are used in the induction phase, the treatment phase can include administration of an additional therapeutic agent, e.g., a PD-1 antibody (e.g., pembrolizumab) or a PD-L1 antibody (e.g., atezolizumab). In some embodiments, however, induction therapy is not administered, and the treatment phase refers to the administration of all agents (e.g., an inhibitor of PI3 kinase (PI3K)-delta, an anti-CD20 antibody, and an anti-PD1 or anti-PD-L1 antibody) in combination. The “treatment phase” is also called “consolidation,” “consolidation therapy,” or “intensification therapy.”

A “maintenance phase” or “maintenance therapy,” as used herein, refers to a phase that occurs subsequent to a treatment phase, as described herein. In the maintenance phase, a patient is given treatment in order to help keep the hematological cancer from returning following successful treatment. Maintenance therapy may include treatment with the same agents that were used previously in the treatment phase. The agents in the maintenance phase may be administered for an extended period of time.

All numbers in this disclosure indicating amounts, ratios, physical properties of materials, and/or use are to be understood as modified by the word “about,” except as otherwise indicated. The term “about,” when referring to a number or numerical range, means that the number or range referred to is an approximation, e.g., within experimental variability (or within statistical experimental error), and thus, the number or numerical range can vary from, e.g., between 1% and 15% of the stated number or numerical range.

The compounds of formula A described herein can contain one or more asymmetric centers (chiral centers) and can thus give rise to enantiomers, diastereomers, and other stereoisomeric forms, in terms of absolute stereochemistry, such as (R)- or (S)-. The present disclosure is meant to encompass all such possible forms, as well as their racemic and resolved forms and mixtures thereof. The individual enantiomers can be separated according to methods known in the art in view of the present disclosure.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive.

The term “resolution” refers to the separation, concentration or depletion of one of the two enantiomeric forms of a molecule.

The present disclosure encompasses solvates of compounds of formula A. Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule, e.g., a disolvate, monosolvate, or hemisolvate, where the ratio of solvent molecule to compound of the present disclosure is about 2:1, about 1:1, or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the invention can be present as solvated forms with a pharmaceutically acceptable solvent, such as water, methanol, ethanol, and the like, and it is intended that the disclosure includes both solvated and unsolvated forms of compounds of the invention. One type of solvate is a hydrate. A “hydrate” relates to a particular subgroup of solvates where the solvent molecule is water. Solvates typically can function as pharmacological equivalents. Preparation of solvates is known in the art. See, e.g., M. Caira et al., J. Pharmaceut. Sci., 93(3):601-611 (2004); E.C. van Tonder et al., AAPS Pharm. Sci. Tech. 5(1):Article 12 (2004). A typical, non-limiting, process of preparing a solvate would involve dissolving a compound of the present disclosure in a desired solvent (organic, water, or a mixture thereof) at temperatures about 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

The term “prodrug” refers to a compound, which is an inactive precursor of a compound, converted into its active form in the body by normal metabolic processes. Prodrug design is discussed generally in Hardma, et al. (eds.), Goodman and Gilman's The Pharmacological Basis of Therapeutics, 9th ed., pp. 11-16 (1996). A thorough discussion is provided in Higuchi et al., Prodrugs as Novel Delivery Systems, Vol. 14, ASCD Symposium Series, and in Roche (ed.), Bioreversible Carriers in Drug Design, American Pharmaceutical Association and Pergamon Press (1987). To illustrate, prodrugs can be converted into a pharmacologically active form through hydrolysis of, for example, an ester or amide linkage, thereby introducing or exposing a functional group on the resultant product. The prodrugs can be designed to react with an endogenous compound to form a water-soluble conjugate that further enhances the pharmacological properties of the compound, for example, increased circulatory half-life. Alternatively, prodrugs can be designed to undergo covalent modification on a functional group with, for example, glucuronic acid, sulfate, glutathione, amino acids, or acetate. The resulting conjugate can be inactivated and excreted in the urine, or rendered more potent than the parent compound. High molecular weight conjugates also can be excreted into the bile, subjected to enzymatic cleavage, and released back into the circulation, thereby effectively increasing the biological half-life of the originally administered compound. Prodrugs of the compounds of the invention are intended to be covered within the scope of this invention.

The present disclosure further encompasses pharmaceutically acceptable salts of the compounds of formula A. Examples of pharmaceutically acceptable addition salts include inorganic and organic acid addition salts and basic salts. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate, succinates, palmoates, benzoates, salicylates, ascorbates, glycerophosphates, ketoglutarates and the like; sulfonates such as methanesulfonate, benzenesulfonate, p-toluenesulfonate and the like; salts of natural amino acids such as glycine, alanine, valine, leucine, isoleucine, norleucine, tyrosine, cystine, cysteine, methionine, proline, hydroxy proline, histidine, ornithine, lysine, arginine, and serine; and salts of non-natural amino acids such as D-isomers or substituted amino acids; salts of guanidine; and salts of substituted guanidine wherein the substituents are selected from nitro, amino, alkyl, alkenyl, alkynyl, ammonium or substituted ammonium salts and aluminum salts.

The dosage amounts described herein are expressed in the amount of a free base agent, and does not include the weight of a counterion (e.g., sulfate) or any water or solvent molecules.

The terms “PI3K-delta selective inhibitor,” “PI3K-δ selective inhibitor,” “PI3K-delta inhibitor,” and “PI3K-δ inhibitor,” are used interchangeably herein, and refer to a compound that selectively inhibits the activity of the PI3K-δ isoform more effectively than other isoforms of the PI3K family (α, β, and γ). For instance, a PI3K-δ selective inhibitor can be a compound that exhibits a 50% inhibitory concentration (IC₅₀) with respect to the δ type PI3-kinase that is at least 20-fold, or lower, than the inhibitor's IC₅₀ with respect to the rest of the other types PI3K isoforms (i.e., α, β, and γ).

The terms “synergy” and “synergistic activity,” as used herein, mean that the combined administration of agents as described herein, e.g., a triple combination of an anti-CD20 antibody, a PI3K-delta inhibitor, and an anti-PD1 or anti-PD-L1 antibody, produces a therapeutic measure that is greater than the additive effects of the agents, when each is used alone and/or when two agents are combined.

As used in the present disclosure and claims, the singular forms “a,” “an,” and “the” include plural forms unless the context clearly dictates otherwise. For example, “a cell” includes a single cell as well as a plurality of cells, including mixtures thereof.

II. PI3K-Delta Selective Inhibitor

The present disclosure provides an innovative combination treatment and treatment regimen for patients with hematological malignancies. The combination treatment includes, inter alia, administering to a subject in need thereof a therapeutically effective amount of at least one PI3K-delta selective inhibitor, e.g., a PI3K-delta selective inhibitor of formula A, or a pharmaceutically acceptable salt, solvate, or prodrug thereof.

The phosphoinositide 3-kinases (PI3Ks) are a family of enzymes that regulate diverse biological functions in every cell type by generating phosphoinositide second-messenger molecules. PI3Ks are involved in various cellular functions, including cell proliferation and survival, cell differentiation, intracellular trafficking, and immunity. The PI3K family is comprised of four different classes: Classes I, II, III, and IV. Classes I-III are lipid kinases and Class IV are serine/threonine protein kinases.

The members of the Class I family of PI3Ks are dimers of a regulatory and a catalytic subunit. The Class I family consists of four isoforms, determined by the 110 kDa catalytic subunits α, β, γ and δ. See Engelman, J. A., Nat. Rev. Genet. 7:606-619 (2006). Class I can be subdivided into two subclasses: Class Ia, formed by the combination of p110 α, β, and δ, and a regulatory subunit (p85, p55 or p50); and Class Ib, formed by p110 γ and p101 regulatory subunits. The delta isoform of PI3K is highly expressed in cells of hematopoietic origin, and strongly upregulated, and often mutated in various hematologic malignancies.

One example of a PI3K-delta selective inhibitor is Idelalisib (trade name Zydelig®), which was approved by the FDA in 2014 for the treatment of relapsed CLL (in combination with Rituxan®; see, Furman, R. R. et al., N Eng. J. Med. 370:997-1007 (2014)), relapsed follicular B-cell non-Hodgkin lymphoma (FL), and relapsed small lymphocytic lymphoma (SLL), another type of non-Hodgkin lymphoma See Zydelig® full prescribing information (Gilead Sciences). Idelalisib has a unique and limiting toxicity profile including immune mediated colitis (grade 3≥5%), pneumonitis (grade 3≥4%), and transaminitis (grade 3≥8%). Therefore the FDA's approval of Zydelie® conies with a boxed warning noting the possbility of fatal and serious toxicities including hepatic, severe diarrhea, cohtis, pneumonitis and intestinal perforation. Id.

Another example of a PI3K-delta selective inhibitor is duvelisib (IPI-145). See O'Brian, S. et al., Blood 124:Abstract No. 3334 (2014). Although duvelisib targets both PI3K delta and gamma, at the dose under development (25 mg twice daily), it primarily inhibits just the delta isoform. Id. Another PI3K-delta selective inhibitor is ACP-319 (previously AMG-319). See Lanasa, M. C. et al., Blood 122:Abstract No. 678 (2013). ACP-319 is currently in development by Acerta Pharma B. V. ME-401 is a new oral PI3K-delta selective inhibitor developed by MEI Pharma. See Moreno, O. et al., poster titled “Clinical Pharmacokinetics and Pharmacodynamics of ME-401, an Oral, Potent, and Selective Inhibitor of Phosphatidylinositol 3-Kinase P110δ, Following Single Ascending Administration to Healthy Volunteers,” which was presented at the American Association for Cancer Research (AACR) Annual Meeting, New Orleans (Apr. 16-20, 2016). INCB-50465 is another PI3K-delta selective inhibitor in development by Incyte Corporation that is in Phase I/II clinical trials for the treatment of B-cell malignancies. See Forero-Torres, A. et al., “Preliminary safety, efficacy, and pharmacodynamics of a highly selective PI3Kδ inhibitor, INCB050465, in patients with previously treated B-cell malignancies” (Abstract No. CT056), presented at the AACR Annual Meeting, New Orleans (Apr. 16-20, 2016).

As provided herein, the PI3K-δ selective inhibitor used in the described methods and kits is a compound of formula A:

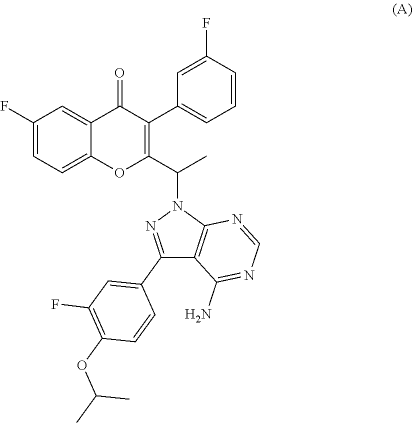 [SMILES: CC(C)Oc1ccc(-c2nn(C(C)c3oc4ccc(F)cc4c(=O)c3-c3cccc(F)c3)c3ncnc(N)c23)cc1F]

or a stereoisomer thereof, or a pharmaceutically acceptable salt, solvate, or prodrug thereof.

In one embodiment, the compound of formula A is (RS)-2-(1-(4-amino-3-(3-fluoro-4-isopropoxyphenyl)-1H-pyrazolo [3,4-d]pyrimidin-1-yl)ethyl-6-fluoro-3-(3-fluorophenyl)-4H-chromen-4-one.

In one embodiment, the compound of formula A is (S)-2-(1-(4-amino-3-(3-fluoro-4-isopropoxyphenyl)-1H-pyrazolo [3,4-d]pyrimidin-1-yl)ethyl-6-fluoro-3-(3-fluorophenyl)-4H-chromen-4-one, or pharmaceutically acceptable salts, solvates, and prodrugs thereof. In another embodiment, the compound of formula A is (R)-2-(1-(4-amino-3-(3-fluoro-4-isopropoxyphenyl)-1H-pyrazolo[3,4-d]pyrimidin-1-yl)ethyl-6-fluoro-3-(3-fluorophenyl)-4H-chromen-4-one, or pharmaceutically acceptable salts, solvates, and prodrugs thereof. The chemical structures of these two compounds are shown below:

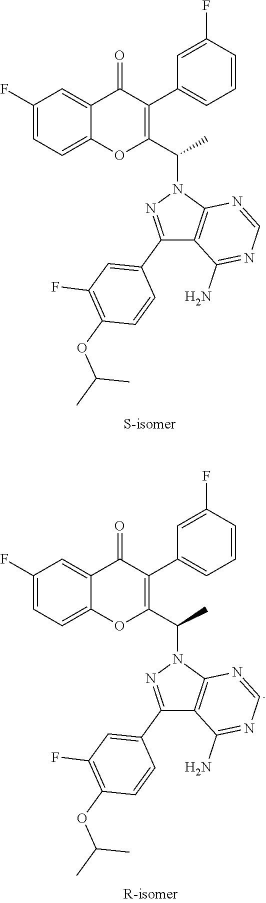 [SMILES: CC(C)Oc1ccc(-c2nn([C@@H](C)c3oc4ccc(F)cc4c(=O)c3-c3cccc(F)c3)c3ncnc(N)c23)cc1F ; CC(C)Oc1ccc(-c2nn([C@H](C)c3oc4ccc(F)cc4c(=O)c3-c3cccc(F)c3)c3ncnc(N)c23)cc1F]

The PI3K-delta inhibitors of formula A can be prepared using the general synthetic methods as disclosed in International Patent Appl. Publ. No. WO 2011/055215 A2 and U.S. Patent Appl. Pub. No. 2011/0118257 A1, each of which is incorporated by reference in its entirety.

In a preferred embodiment, the PI3K-delta inhibitor of Formula A is (S)-2-(1-(4-amino-3-(3-fluoro-4-isopropoxyphenyl)-1H-pyrazolo [3,4-d]pyrim-idin-1-yl)ethyl-6-fluoro-3-(3-fluorophenyl)-4H-chromen-4-one, or a pharmaceutically acceptable salt, solvate, or prodrug thereof. The preparation of (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one and its salts is described in International Publ. No. WO 2014/006572 and U.S. Patent Publ. No. 2014/0011819. In addition to describing the synthesis of (S)-2-(1-(4-amino-3-(3-fluoro-4-isopropoxyphenyl)-1H-pyrazolo [3,4-d]pyrim-idin-1-yl)ethyl-6-fluoro-3-(3-fluorophenyl)-4H-chromen-4-one, WO 2014/006572 and U.S. 2014/0011819 also disclose the therapeutic activity of this molecule to inhibit, regulate and/or modulate the signal transduction of PI3K. This PI3K-delta inhibitor of Formula A is also described in U.S. Pat. No. 9,150,579, which issued October 6, 2015.

In a particularly preferred embodiment, the PI3K-delta inhibitor of Formula A is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one p-toluenesulfonic acid (PTSA) salt, which exhibits enhanced solubility and pharmacokinetics upon oral administration. See International Publ. No. WO 2015/181728. The PTSA salt of (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one is also known as TGR-1202. As used herein, the term “TGR-1202” refers to the PTSA salt of (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one. The generic International Non-Proprietary Name (INN)/U.S.Adopted Name (USAN) of TGR-1202 is umbralisib tosylate. Each of these applications and patents is incorporated by reference in its entirety.

TGR-1202 is a highly specific, orally available PI3K delta inhibitor, targeting the delta isoform with nanomolar inhibitory potency and high selectivity over the α, β, and γ isoforms. The potency of TGR-1202 against human PI3K isoforms in an enzyme based assay is shown in Table 1.

TABLE 1
Potency of TGR-1202 against human PI3K isoforms

| PI3K isoforms (Human) | IC₅₀ (nM) |
|---|---|
| α | >10,000 |
| β | 1,116 |
| γ | 1,065 |
| δ | 22.23 |

The activity of TGR-1202 was evaluated in a single-agent Phase I dose-escalation study in patients with relapsed and refractory hematologic malignancies (see e.g., Burris, H. A. et al., J. Clinical Oncology (ASCO Annual Meeting Abstracts) 32 (15): 2513 (2014)). Burris reported that TGR-1202 was well-tolerated in patients with relapsed or refractory hematologic malignancies with no reported hepatic toxicity and signs of clinical activity at doses ≥800 mg each day.

In some embodiments, for the methods and kits described herein, the PI3K-delta inhibitor of formula A (e.g., TGR-1202) is administered to a subject daily at a dosage from: about 200 mg to about 1200 mg, about 400 mg to about 1000 mg, about 400 mg to about 800 mg, about 400 mg, about 500 mg, about 600 mg, about 700 mg, about 800 mg, about 900 mg, about 1000 mg, or about 1200 mg.

In some embodiments, the PI3K-delta inhibitor of formula A is formulated for oral administration. In some embodiments, TGR-1202, or a pharmaceutically acceptable salt, solvate or prodrug thereof, is formulated for oral administration. In some embodiments, TGR-1202, or a pharmaceutically acceptable salt, solvate or prodrug thereof, is administered to a patient under a fed condition.

In general, administration of TGR-1202 under fed conditions results in a higher bioavailability (e.g., increased AUC and Cₘₐₓ) relative to administration under fasting conditions, as illustrated in Table 2 below.

TABLE 2
Comparison of orally administering TGR-1202 under fasted and
fed conditions (single oral dose of 200 mg)

| | Geometric | | | |
|---|---|---|---|---|
| | LS Means | | % Geometric | |
| Parameters | Fasting | Fed | Mean Ratio | Confidence Interval |
| AUC₀₋ₜ | 6029.87 | 9692.02 | 160.73 | 140.25-184.21 |
| (ng · hr/mL) | | | | |
| AUC_{0-inf} | 8391.35 | 14047.17 | 167.40 | 141.59-197.92 |
| (ng · hr/mL) | | | | |
| Cₘₐₓ (ng/mL) | 176.78 | 483.15 | 273.31 | 234.04-319.17 |

In some embodiments, for the methods and kits described herein, the PI3K-delta inhibitor is micronized. In some embodiments, TGR-1202, or a pharmaceutically acceptable salt, solvate or prodrug thereof, is micronized.

Administration of micronized TGR-1202 results in a higher bioavailability (e.g., increased AUC and Cₘₐₓ) relative to administration of non-micronized TGR-1202, as illustrated in Table 3 below.

TABLE 3
Comparison of micronized and non-micronized TGR-1202 formulations
(single oral dose of 200 mg)

| | Geometric LS Means | | % | |
|---|---|---|---|---|
| | Non- | | Geometric | |
| | Micronized | Micronized | Mean | Confidence |
| Parameters | Formulation | Formulation | Ratio | Interval |
| AUC₀₋ₜ | 5906.11 | 9439.82 | 159.83 | 149.43-170.95 |
| (ng · hr/mL) | | | | |
| AUC_{0-inf} | 7715.67 | 12378.19 | 160.43 | 146.49-175.70 |
| (ng · hr/mL) | | | | |
| Cₘₐₓ (ng/mL) | 166.20 | 371.70 | 223.65 | 202.33-247.20 |

TGR-1202 is not associated with treatment related transaminitis or colitis, which distinguishes TGR-1202 from idelalisib. The difference in toxicity profiles between TGR-1202 and idelalisib is of profound significance when TGR-1202 is administered in combination with an immune check point inhibitor, such as a PD-1 or PD-L1 antibody, in the management of hematological cancers.

In some embodiments, for the methods and kits described herein, TGR-1202 or a pharmaceutically acceptable salt, solvate or prodrug thereof, is administered at a dose from: about 200 to about 1200 mg, about 400 to about 1000 mg, about 500 to about 800 mg, about 500 to about 1000 mg, about 600 to about 800 mg, about 600 to about 1000 mg, about 600 mg, about 700 mg, about 800 mg, about 900 mg, about 1000 mg, or about 1200 mg per day.

In some embodiments, TGR-1202, or a pharmaceutically acceptable salt, solvate or prodrug thereof, is administered at a dose from about 400 mg to about 800 mg per day. In some embodiments, TGR-1202, or a pharmaceutically acceptable salt, solvate or prodrug thereof, is administered at a dose of about 400 mg, about 600 mg or about 800 mg per day. One skilled in the art will appreciate that the dosage of TGR-1202 (or a pharmaceutically acceptable salt, solvate, or prodrug thereof) and/or frequency of administering TGR-1202 (or a pharmaceutically acceptable salt, solvate or prodrug thereof) may change during the course of therapy (lowered or increased) depending upon the patient's clinical response, side effects, etc., or during different phases of therapy (i.e., induction, treatment, or maintenance).

III. Anti-CD20 Antibodies

The present disclosure provides an innovative combination treatment and treatment regimen for patients with hematological cancers. The combination treatment includes, inter alia, administering to a subject in need thereof a therapeutically effective amount of at least one anti-CD20 antibody (e.g., ublituximab).

CD20 is a hydrophobic transmembrane phosphoprotein that is expressed predominantly in pre-B cells and mature peripheral B cells in humans and mice. In humans, CD20 is also strongly and homogeneously expressed in most mature B-cell malignancies, including, for example, most non-Hodgkin's B-cell lymphomas (NHL) and B-type Chronic Lymphocytic Leukemia's (B-CLL). The CD20 antigen is not expressed on haematopoietic stem cells or on plasmocytes.

Anti-CD20 monoclonal antibodies have been, and continue to be, developed for the treatment of B-cell diseases. The chimeric anti-CD20 monoclonal antibody rituximab (Rituxan®) has become the standard therapy for many CD20-positive B-cell lymphomas and was the first mAb approved for any oncology indication. Demarest, S. J. et al., mAbs 3:338-351 (2011). However, there are a substantial number of patients who are refractory to treatment with rituximab or who develop resistance in the course of prolonged treatment with rituximab (used as a single agent or even in combination with chemotherapeutic regimens).

Aside from rituximab, a number of other anti-CD20 antibodies are also known in the art, including for example, ublituximab (TG-1101), ofatumumab (HuMax; Intracel), ocrelizumab, veltuzumab, GA101 (obinutuzumab), AME-133v (Applied Molecular Evolution), ocaratuzumab (Mentrik Biotech), PRO131921, tositumomab, ibritumomab-tiuxetan, hA20 (Immunomedics, Inc.), BLX-301 (Biolex Therapeutics), Reditux (Dr. Reddy's Laboratories), and PRO70769 (described in WO2004/056312).

Rituximab is a genetically engineered chimeric murine/human monoclonal antibody directed against the CD20 antigen. Rituximab is the antibody called “C2B8” in U.S. Pat. No. 5,736,137. The amino acid sequence of rituximab antibody and exemplary methods for its production via recombinant expression in Chinese Hamster Ovary (CHO) cells are disclosed in U.S. Pat. No. 5,736,137, which is herein incorporated by reference in its entirety. Rituximab was initially approved by the FDA in 1997 for treating non-Hodgkin's lymphoma. Rituximab is commercially available as Rituxan®.

Ofatumumab is an anti-CD20 IgG1κ human monoclonal antibody. Studies indicated that ofatumumab dissociates from CD20 at a slower rate compared to the rituximab and binds a membrane-proximal epitope. Zhang et al., Mabs 1: 326-331 (2009). Epitope mapping has indicated that ofatumumab binds an epitope located closer to the N-terminus of CD20 compared to the location targeted by rituximab and includes an extracellular loop of the antigen. Id.

Ublituximab (also known as UBX, UTX, TG-1101, TGTX-1101, Utuxin™, LFB-R603, TG20, EMAB603) is a monoclonal antibody that targets a specific and unique epitope on CD20 and that has been bioengineered for enhanced clinical activity and potency. See, Miller et al., Blood 120: Abstract No. 2756 (2012); Deng, C. et. al. J. Clin Oncol 31:Abstract No. 8575 (2013); and O'Connor, O. A. et al., “A phase I trial of ublituximab (TG-1101), a novel glycoengineered anti-CD20 monoclonal antibody (mAb) in B-cell non-Hodgkin lymphoma patients with prior exposure to rituximab,” J. Clin. Oncol. 32:5s (2014), (suppl; Abstract No. 8524). Ublituximab is also described in U.S. Pat. No. 9,234,045. Ublituximab was engineered for potent activity, exhibiting a unique amino acid sequence and allowing a low fucose content, designed to induce superior antibody-dependent cell-mediated cytotoxicity (ADCC). Ublituximab has been studied in a variety of patient populations (e.g., NHL, CLL), both as a single agent, and in combination with other agents. For example, O'Connor et al., supra, showed that single-agent ublituximab was well-tolerated and active in rituxin-exposed patients. In a phase I trial, Lunning, M. et al., American Society of Hematology Annual Meeting and Exposition, Dec. 5-8, 2015, Abstract No. 1538, showed that ublituximab and TGR-1202 demonstrated activity and a favorable safety profile in relapsed/refractory B-cell NHL and high-risk CLL. And in a phase II trial, Sharman J. et. al., American Society of Hematology Annual Meeting and Exposition, Dec. 5-8, 2015, Abstract No. 3980, showed that ublituximab in combination with Ibrutinib was highly active in patients with relapsed and/or refractory mantle cell lymphoma.

In a preferred embodiment, the anti-CD20 antibody used in the methods (and kits) described herein is ublituximab or an anti-CD20 antibody that binds to the same epitope as ublituximab. In a particularly preferred embodiment, the anti-CD20 antibody is ublituximab.

In some embodiments, the ublituximab comprises the VH CDR1, CDR2, and CDR3 region of sequences SEQ ID NOS: 1, 2, and 3, and the VL CDR1, CDR2, and CDR3 region of sequences SEQ ID NOS: 6, 7, and 8. In some embodiments, the ublituximab comprises the VH of SEQ ID NO: 4 and the VL of SEQ ID NO: 9.

Ublituximab comprises the antibody sequences provided below:

Variable heavy chain (VH) CDR1:
(SEQ ID NO: 1)
Gly Tyr Thr Phe Thr Ser Tyr Asn
Variable heavy chain (VH) CDR2:
(SEQ ID NO: 2)
Ile Tyr Pro Gly Asn Gly Asp Thr
Variable heavy chain (VH) CDR3:
(SEQ ID NO: 3)
Ala Arg Tyr Asp Tyr Asn Tyr Ala Met Asp Tyr
Variable heavy chain (VH):
(SEQ ID NO: 4)
Gln Ala Tyr Leu Gln Gln Ser Gly Ala Glu Leu Val Arg Pro Gly Ala Ser
Val Lys Met Ser Cys Lys Ala Ser Gly Tyr Thr Phe Thr Ser Tyr Asn Met
His Trp Val Lys Gln Thr Pro Arg Gln Gly Leu Glu Trp Ile Gly Gly Ile
Tyr Pro Gly Asn Gly Asp Thr Ser Tyr Asn Gln Lys Phe Lys Gly Lys Ala
Thr Leu Thr Val Gly Lys Ser Ser Ser Thr Ala Tyr Met Gln Leu Ser Ser
Leu Thr Ser Glu Asp Ser Ala Val Tyr Phe Cys Ala Arg Tyr Asp Tyr Asn
Tyr Ala Met Asp Tyr Trp Gly Gln Gly Thr Ser Val Thr Val Ser Ser
Constant heavy chain:
(SEQ ID NO: 5)
Ala Ser Thr Lys Gly Pro Ser Val Phe Pro Leu Ala Pro Ser Ser Lys Ser
Thr Ser Gly Gly Thr Ala Ala Leu Gly Cys Leu Val Lys Asp Tyr Phe Pro
Glu Pro Val Thr Val Ser Trp Asn Ser Gly Ala Leu Thr Ser Gly Val His
Thr Phe Pro Ala Val Leu Gln Ser Ser Gly Leu Tyr Ser Leu Ser Ser Val
Val Thr Val Pro Ser Ser Ser Leu Gly Thr Gln Thr Tyr Ile Cys Asn Val
Asn His Lys Pro Ser Asn Thr Lys Val Asp Lys Lys Val Glu Pro Lys Ser
Cys Asp Lys Thr His Thr Cys Pro Pro Cys Pro Ala Pro Glu Leu Leu Gly
Gly Pro Ser Val Phe Leu Phe Pro Pro Lys Pro Lys Asp Thr Leu Met Ile
Ser Arg Thr Pro Glu Val Thr Cys Val Val Val Asp Val Ser His Glu Asp
Pro Glu Val Lys Phe Asn Trp Tyr Val Asp Gly Val Glu Val His Asn Ala
Lys Thr Lys Pro Arg Glu Glu Gln Tyr Asn Ser Thr Tyr Arg Val Val Ser
Val Leu Thr Val Leu His Gln Asp Trp Leu Asn Gly Lys Glu Tyr Lys Cys
Lys Val Ser Asn Lys Ala Leu Pro Ala Pro Ile Glu Lys Thr Ile Ser Lys
Ala Lys Gly Gln Pro Arg Glu Pro Gln Val Tyr Thr Leu Pro Pro Ser Arg
Asp Glu Leu Thr Lys Asn Gln Val Ser Leu Thr Cys Leu Val Lys Gly Phe
Tyr Pro Ser Asp Ile Ala Val Glu Trp Glu Ser Asn Gly Gln Pro Glu Asn
Asn Tyr Lys Thr Thr Pro Pro Val Leu Asp Ser Asp Gly Ser Phe Phe Leu
Tyr Ser Lys Leu Thr Val Asp Lys Ser Arg Trp Gln Gln Gly Asn Val Phe
Ser Cys Ser Val Met His Glu Ala Leu His Asn His Tyr Thr Gln Lys Ser
Leu Ser Leu Ser Pro Gly Lys
Variable light chain (VL) CDR1:
(SEQ ID NO: 6)
Ser Ser Val Ser Tyr
Variable light chain (VL) CDR2:
(SEQ ID NO: 7)
Ala Thr Ser
Variable light chain (VL) CDR3:
(SEQ ID NO: 8)
Gln Gln Trp Thr Phe Asn Pro Pro Thr
Variable light chain (VL):
(SEQ ID NO: 9)
Gln Ile Val Leu Ser Gln Ser Pro Ala Ile Leu Ser Ala Ser Pro Gly Glu
Lys Val Thr Met Thr Cys Arg Ala Ser Ser Ser Val Ser Tyr Met His Trp
Tyr Gln Gln Lys Pro Gly Ser Ser Pro Lys Pro Trp Ile Tyr Ala Thr Ser
Asn Leu Ala Ser Gly Val Pro Ala Arg Phe Ser Gly Ser Gly Ser Gly Thr
Ser Tyr Ser Phe Thr Ile Ser Arg Val Glu Ala Glu Asp Ala Ala Thr Tyr
Tyr Cys Gln Gln Trp Thr Phe Asn Pro Pro Thr Phe Gly Gly Gly Thr Arg
Leu Glu Ile Lys
Constant light chain:
(SEQ ID NO: 10)
Thr Val Ala Ala Pro Ser Val Phe Ile Phe Pro Pro Ser Asp Glu Gln Leu
Lys Ser Gly Thr Ala Ser Val Val Cys Leu Leu Asn Asn Phe Tyr Pro Arg
Glu Ala Lys Val Gln Trp Lys Val Asp Asn Ala Leu Gln Ser Gly Asn Ser
Gln Glu Ser Val Thr Glu Gln Asp Ser Lys Asp Ser Thr Tyr Ser Leu Ser
Ser Thr Leu Thr Leu Ser Lys Ala Asp Tyr Glu Lys His Lys Val Tyr Ala
Cys Glu Val Thr His Gln Gly Leu Ser Ser Pro Val Thr Lys Ser Phe Asn
Arg Gly Glu Cys

In some embodiments, in the methods and kits described herein, ublituximab is administered at a dose from: about 450 mg to about 1200 mg, about 500 to about 1200 mg, about 600 to about 1200 mg, about 500 to about 1000 mg, about 600 to about 1000 mg, about 600 to about 900 mg, about 500 mg, about 600 mg, about 700 mg, about 800 mg, or about 900 mg. In certain embodiments, the ublituximab is administered at a dose of about 900 mg.

Ublituximab can be administered about once every 1 to 9 weeks, about once every week, about twice every week, about once every 2 weeks, about once every 3 weeks, about once every 4 weeks, about once every 5 weeks, about once every 6 weeks, about once every 7 weeks, about once every 8 week, or about once every 9 weeks. One skilled in the art will appreciate that the dosage of ublituximab and/or frequency of administering ublituximab may change during the course of therapy (lowered or increased) depending upon the patient's clinical response, side effects, etc.

In some embodiments, in the treatment phase, ublituximab is administered at a dose from: about 450 to about 1200 mg, about 450 to about 1000 mg, about 500 to about 1200 mg, about 500 to about 1000 mg, about 500 to about 900 mg, about 600 to about 1200 mg, about 600 to about 1000 mg, about 600 to about 900 mg, about 500 mg, about 600 mg, about 700 mg, about 750 mg, about 800 mg, about 900 mg, about 1000 mg, about 1100 mg, or about 1200 mg about once every 4 to 7 weeks, about once every 5 to 7 weeks, about once every 5 to 6 weeks, about once a week, about once every 2 weeks, about once every 3 weeks, about once every 4 weeks, about once every 5 weeks, about once every 6 weeks, or about once every 7 weeks.

In some embodiments, the methods and kits described herein comprise an induction phase prior to the treatment phase. In the induction phase, ublituximab is administered at a dose from: about 450 to about 1200 mg, about 450 to about 1000 mg, about 500 to about 1200 mg, about 500 to about 1000 mg, about 500 to about 900 mg, about 600 to about 1200 mg, about 600 to about 1000 mg, about 600 to about 900 mg, about 500 mg, about 600 mg, about 700 mg, about 800 mg, about 900 mg, about 1000 mg, about 1100 mg, or about 1200 mg about once every 1 to 3 weeks, about once every 2 to 3 weeks, about once every 1 to 2 weeks, about once every 1 week, about once every 2 weeks, or about once every 3 weeks.

One skilled in the art will appreciate that the dosage of ublituximab and/or frequency of administering ublituximab may change during the course of therapy (lowered or increased) depending upon the patient's clinical response, side effects, etc., during the treatment phase and/or induction phase.

In some embodiments, the ublituximab is formulated and/or administered intravenously, preferably by infusion.

In some embodiments, the anti-CD20 antibody or fragment thereof binds to the same epitope as ublituximab. In some embodiments, the anti-CD20 antibody or fragment thereof binds to a sequence comprising amino acids N153-S179 of CD20. In some embodiments, the anti-CD20 antibody or fragment thereof binds to a discontinuous epitope in amino acids N153-S179 of CD20.

In some embodiments, the anti-CD20 antibody or fragment thereof binds to CD20 with an affinity characterized by a dissociation constant KD of less than about 10⁻⁷ M, less than about 10⁻⁸ M or less than about 10⁻⁹ M. In some embodiments, the anti-CD20 antibody or fragment thereof binds to CD20 with an affinity characterized by a dissociation constant KD of 10⁻¹⁰ to 10⁻⁹ M. In some embodiments the anti-CD20 antibody or fragment thereof binds to CD20 with an affinity characterized by a dissociation constant KD of 0.7×10⁻⁹ M. As used in the context of antibody binding dissociation constants, the term “about” allows for the degree of variation inherent in the methods utilized for measuring antibody affinity. For example, depending on the level of precision of the instrumentation used, standard error based on the number of samples measured, and rounding error, the term “about 10⁻² M” might include, for example, from 0.05 M to 0.005 M.

In some embodiments, the anti-CD20 antibody exhibits a high affinity to Fc-gammaRIII (CD16). In some embodiments, as a result of their high affinity for the Fc region of the antibody to CD16, such antibodies are not displaced by IgG polyclonal antibodies, especially by IgG present in blood serum. In some embodiments the antibody binds to CD16 (e.g., expressed on a macrophage) with an affinity of at least 2×10⁶ M⁻¹, at least 2×10⁷ M⁻¹, 2×10⁸ M⁻¹ or 2×10⁷ M⁻¹, e.g., as determined by Scatchard analysis or BlAcore technology (Label-free surface plasmon resonance based technology).

In some embodiments, the anti-CD20 antibody is glycoengineered. As used herein, a “glycoengineered” anti-CD20 antibody means that the sugar molecules (N-glycan) in the Fc region of the antibody have been altered or engineered, either genetically, enzymatically, chemically, or selected for during the manufacturing process. in order to, e.g., increase the affinity of the antibody for Fc receptors on effector cells and/or to reduce its specific carbohydrate content in its Fc region.

In some embodiments, the anti-CD20 antibody exhibits a glycosylation pattern characterized by low fucose content in its Fc region. For example, in some embodiments, a composition comprises anti-CD20 antibodies in which the antibodies comprise N-glycoside-linked sugar chains bound on the Fc-gamma glycosylation site (Asn 297, EU numbering), wherein among the N-glycoside-linked sugar chains of all the antibodies of the composition, the fucose content is less than 65%, less than 60%, less than 55%, less than 50%, less than 45%, or less than 40%. In some embodiments, among the N-glycoside-linked sugar chains of all the antibodies of the composition, the fucose content is 15 to 45% or 20 to 40%.

In some embodiments, the anti-CD20 antibody exhibits potent in vitro antibody-dependent cellular cytotoxicity (ADCC) and can be said to be “ADCC-optimized”. In some embodiments, the anti-CD20 antibody produces an ADCC plateau of at least about 10%, at least about 15%, at least about 20%, at least about 25%, or at least about 30% at a concentration of 50 ng/ml using natural killer (NK) cells from healthy donors. Techniques for measuring ADCC are known in the art and provided, for example, in de Romeuf, C. et al., British Journal of Haematology 140: 635-643 (2008). In some embodiments, the anti-CD20 antibody produces an ADCC plateau at about 35% at a concentration of 50 ng/ml using NK cells from healthy donors.

In some embodiments, the anti-CD20 antibody can decrease NF-kappa-B activity. In some embodiments, the anti-CD20 antibody can decrease SNAIL expression. In some embodiments, the anti-CD20 antibody can increase RKIP activity. In some embodiments, the anti-CD20 antibody can increase PTEN activity. In some embodiments, the anti-CD20 antibody can increase sensitization of a cell to TRAIL-apoptosis.

In some embodiments, the anti-CD20 antibody is Fc-gamma-RIIIA (CD16) optimized. Antibodies capable of activating type III Fc receptors and having a particular glycan structure have been described, for example, in U.S. Pat. No. 7,931,895, which is herein incorporated by reference in its entirety. Thus, in some embodiments, the anti-CD20 antibody is modified on Asn 297 (EU numbering) with N-glycosylations of the bi-antennary and/or oligomannoside type as described in U.S. Pat. No. 7,931,895. Methods of producing antibodies with strong affinity for receptor CD16 of the effector cells of the immune system are provided, for example, in U.S. Published Appl. No. 2005/0271652, which is herein incorporated by reference in its entirety.

In some embodiments, the anti-CD20 antibody has high ADCC activity. Methods of producing antibodies with high ADCC activity are provided, for example, in U.S. Pat. No. 7,713,524, which is herein incorporated by reference in its entirety.

Thus, in some embodiments, an isolated antibody or antigen-binding fragment, variant, or derivative thereof comprises, consists essentially of, or consists of an immunoglobulin heavy chain variable domain (VH domain), wherein at least one (i.e., one, two, or three) of the CDRs of the VH domain has an amino acid sequence that is at least 80%, 85%, 90%, 95%, 96%, 97%, 98%, 99%, or identical to the CDR1, CDR2, or CDR3 region of sequences SEQ ID NO:1, 2, or 3, wherein an antibody or antigen-binding fragment thereof comprising the VH domain can specifically or preferentially bind to CD20.

In another embodiment, an isolated antibody or antigen-binding fragment, variant, or derivative thereof comprises, consists essentially of, or consists of an immunoglobulin heavy chain variable domain (VH domain), wherein at least one (i.e., one, two, or three) of the CDRs of the VH domain has an amino acid sequence identical, except for 1, 2, 3, 4, or 5 conservative amino acid substitutions, to the CDR1, CDR2, or CDR3 region of sequences SEQ ID NO:1, 2, or 3, wherein an antibody or antigen-binding fragment, variant, or derivative thereof comprising the VH domain can specifically or preferentially bind to CD20.

In another embodiment, an isolated antibody or antigen-binding fragment, variant, or derivative thereof comprises, consists essentially of, or consists of a VH domain that has an amino acid sequence that is at least 80%, 85%, 90%, 91%, 92%, 93%, 94%, 95%, 96%, 97%, 98%, 99%, or 100% identical to a VH amino acid sequence of SEQ ID NO:4, wherein an antibody or antigen-binding fragment, variant, or derivative thereof comprising the VH domain can specifically or preferentially bind to CD20.

In another embodiment, an isolated antibody or antigen-binding fragment, variant, or derivative thereof comprises, consists essentially of, or consists of a heavy chain that has an amino acid sequence that is at least 80%, 85%, 90%, 91%, 92%, 93%, 94%, 95%, 96%, 97%, 98%, 99%, or 100% identical to a heavy chain amino acid sequence comprising SEQ ID NOs: 4 and 5, wherein an antibody or antigen-binding fragment, variant, or derivative thereof comprising the heavy chain can specifically or preferentially bind to CD20.

In some embodiments, an isolated antibody or antigen-binding fragment, variant, or derivative thereof comprises, consists essentially of, or consists of an immunoglobulin light chain variable domain (VL domain), wherein at least one (i.e., one, two, or three) of the CDRs of the VL domain has an amino acid sequence that is at least 80%, 85%, 90%, 95%, 96%, 97%, 98%, 99%, or identical to the CDR1, CDR2, or CDR3 region of sequences SEQ ID NO:6, 7, or 8, wherein an antibody or antigen-binding fragment thereof comprising the VL domain can specifically or preferentially bind to CD20.

In another embodiment, an isolated antibody or antigen-binding fragment, variant, or derivative thereof comprises, consists essentially of, or consists of an immunoglobulin light chain variable domain (VL domain), wherein at least one (i.e., one, two, or three) of the CDRs of the VL domain has an amino acid sequence identical, except for 1, 2, 3, 4, or 5 conservative amino acid substitutions, to the CDR1, CDR2, or CDR3 region of SEQ ID NO:6, 7, or 8, wherein an antibody or antigen-binding fragment, variant, or derivative thereof comprising the VL domain can specifically or preferentially bind to CD20.

In another embodiment, an isolated antibody or antigen-binding fragment, variant, or derivative thereof comprises, consists essentially of, or consists of a VL domain that has an amino acid sequence that is at least 80%, 85%, 90%, 91%, 92%, 93%, 94%, 95%, 96%, 97%, 98%, 99%, or 100% identical to a VL amino acid sequence of SEQ ID NO:9, wherein an antibody or antigen-binding fragment, variant, or derivative thereof comprising the VL domain can specifically or preferentially bind to CD20.

In another embodiment, an isolated antibody or antigen-binding fragment, variant, or derivative thereof comprises, consists essentially of, or consists of a light chain that has an amino acid sequence that is at least 80%, 85%, 90%, 91%, 92%, 93%, 94%, 95%, 96%, 97%, 98%, 99%, or 100% identical to a light chain amino acid sequence comprising SEQ ID NOs:9 and 10, wherein an antibody or antigen-binding fragment, variant, or derivative thereof comprising the light chain can specifically or preferentially bind to CD20.

In some embodiments, an isolated antibody or antigen-binding fragment, variant, or derivative thereof comprises, consists essentially of, or consists of an immunoglobulin heavy chain variable domain (VH domain) and an immunoglobulin light chain variable domain (VL domain), wherein at least one (i.e., one, two, or three) of the CDRs of the VH domain has an amino acid sequence that is at least 80%, 85%, 90%, 95%, 96%, 97%, 98%, 99%, or identical to the CDR1, CDR2, or CDR3 region of sequences SEQ ID NO:1, 2, or 3, wherein at least one (i.e., one, two, or three) of the CDRs of the VL domain has an amino acid sequence that is at least 80%, 85%, 90%, 95%, 96%, 97%, 98%, 99%, or identical to the CDR1, CDR2 or CDR3 region of sequences SEQ ID NO:6, 7, or 8, and wherein an antibody or antigen-binding fragment thereof comprising the VH domain and VL domain can specifically or preferentially bind to CD20.

In another embodiment, an isolated antibody or antigen-binding fragment, variant, or derivative thereof comprises, consists essentially of, or consists of an immunoglobulin heavy chain variable domain (VH domain), and an immunoglobulin light chain variable domain (VL domain), wherein at least one (i.e., one, two, or three) of the CDRs of the VH domain has an amino acid sequence identical, except for 1, 2, 3, 4, or 5 conservative amino acid substitutions, to the CDR1, CDR2, or CDR3 region of sequences SEQ ID NO:1, 2, or 3, wherein at least one (i.e., one, two, or three) of the CDRs of the VL domain has an amino acid sequence identical, except for 1, 2, 3, 4, or 5 conservative amino acid substitutions, to the CDR1, CDR2 or CDR3 region of SEQ ID NO:6, 7, or 8, and wherein an antibody or antigen-binding fragment, variant, or derivative thereof comprising the VH and VL can specifically or preferentially bind to CD20.

In some embodiments, the anti-CD20 antibody or antigen-binding fragment, variant, or derivative thereof comprises the VH CDR1, CDR2, and CDR3 region of sequences SEQ ID NO:1, 2, and 3, and the VL CDR1, CDR2, and CDR3 region of sequences SEQ ID NO:6, 7, and 8.

In another embodiment, an isolated antibody or antigen-binding fragment, variant, or derivative thereof comprises, consists essentially of, or consists of a VH domain and a VL domain, wherein the VH has an amino acid sequence that is at least 80%, 85%, 90%, 91%, 92%, 93%, 94%, 95%, 96%, 97%, 98%, 99%, or 100% identical to a VH amino acid sequence of SEQ ID NO:4, wherein the VL domain that has an amino acid sequence that is at least 80%, 85%, 90%, 91%, 92%, 93%, 94%, 95%, 96%, 97%, 98%, 99%, or 100% identical to a VL amino acid sequence of SEQ ID NO:9, and wherein an antibody or antigen-binding fragment, variant, or derivative thereof comprising the VH domain and VL domain can specifically or preferentially bind to CD20.

In some embodiments, the anti-CD20 antibody or antigen-binding fragment thereof comprises the VH of SEQ ID NO:4 and the VL of SEQ ID NO:9.

In some embodiments, the anti-CD20 antibody or antigen-binding fragment thereof binds to the same epitope as an antibody comprising the VH of SEQ ID NO:4 and the VL of SEQ ID NO:9.

In another embodiment, an isolated antibody or antigen-binding fragment, variant, or derivative thereof comprises, consists essentially of, or consists of a heavy chain and a light chain, wherein the heavy chain has an amino acid sequence that is at least 80%, 85%, 90%, 91%, 92%, 93%, 94%, 95%, 96%, 97%, 98%, 99%, or 100% identical to a heavy chain amino acid sequence comprising SEQ ID NOs: 4 and 5, wherein the light chain has an amino acid sequence that is at least 80%, 85%, 90%, 91%, 92%, 93%, 94%, 95%, 96%, 97%, 98%, 99%, or 100% identical to a light chain amino acid sequence comprising SEQ ID NOs: 9 and 10, and wherein an antibody or antigen-binding fragment, variant, or derivative thereof comprising the heavy chain and light chain can specifically or preferentially bind to CD20.

In some embodiments, the anti-CD20 antibody or antigen-binding fragment thereof comprises a heavy chain comprising SEQ ID NOs: 4 and 5 and a light chain comprising SEQ ID NOs: 9 and 10.

In some embodiments, the anti-CD20 antibody or antigen-binding fragment thereof binds to the same epitope as an antibody comprising SEQ ID NO:4 and SEQ ID NO:5.

In some embodiments, the anti-CD20 antibody is ublituximab.

In some embodiments, the anti-CD20 antibody is EMAB603 (see WO2006/064121, which is herein incorporated by reference in its entirety), produced by the clone R603-12D11, and deposited to the Collection Nationale des Cultures de Microorganismes under the accession number CNCM 1-3529.

In some embodiments, the anti-CD20 antibody is produced in the rat hybridoma YB2/0 cell line (cell YB2/3HL.P2.G11.16Ag.20, registered at the American Type Culture Collection (ATCC) under ATCC number CRL-1662).

The precise chemical structure of an antibody capable of specifically binding CD20 and retaining the desired activity depends on a number of factors. As ionizable amino and carboxyl groups are present in the molecule, a particular polypeptide can be obtained as an acidic or basic salt, or in neutral form. All such preparations that retain their biological activity when placed in suitable environmental conditions are included in the definition of anti-CD20 antibodies as used herein. Further, the primary amino acid sequence of the antibody can be augmented by derivatization using sugar moieties (glycosylation) or by other supplementary molecules such as lipids, phosphate, acetyl groups and the like. It can also be augmented by conjugation with saccharides. Certain aspects of such augmentation are accomplished through post-translational processing systems of the producing host; other such modifications can be introduced in vitro. In any event, such modifications are included in the definition of an anti-CD20 antibody used herein so long as the desired properties of the anti-CD20 antibody are not destroyed. It is expected that such modifications can quantitatively or qualitatively affect the activity, either by enhancing or diminishing the activity of the polypeptide, in the various assays. Further, individual amino acid residues in the chain can be modified by oxidation, reduction, or other derivatization, and the polypeptide can be cleaved to obtain fragments that retain activity. Such alterations that do not destroy the desired properties (e.g., binding specificity for CD20) do not remove the polypeptide sequence from the definition of anti-CD20 antibodies of interest as used herein.

The art provides substantial guidance regarding the preparation and use of polypeptide variants. In preparing variants of an anti-CD20 binding molecule, e.g., an antibody or antigen-binding fragment, variant, or derivative thereof, one of skill in the art can readily determine which modifications to the native protein's nucleotide or amino acid sequence will result in a variant that is suitable for use as a therapeutically active component of a pharmaceutical composition.

It is possible to introduce mutations only in framework regions or only in CDR regions of an antibody molecule. Introduced mutations can be silent or neutral missense mutations, i.e., have no, or little, effect on an antibody's ability to bind antigen. These types of mutations can be useful to optimize codon usage, or improve a hybridoma's antibody production. Alternatively, non-neutral missense mutations can alter an antibody's ability to bind antigen. The location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen-binding activity or alteration in binding activity (e.g., improvements in antigen-binding activity or change in antibody specificity). Following mutagenesis, the encoded protein can routinely be expressed and the functional and/or biological activity of the encoded protein, (e.g., ability to immunospecifically bind at least one epitope of a CD20 polypeptide) can be determined using techniques described herein or by routinely modifying techniques known in the art.

In certain embodiments, the anti-CD20 antibodies comprise at least one optimized complementarity-determining region (CDR). By “optimized CDR” is intended that the CDR has been modified and optimized sequences selected based on the sustained or improved binding affinity and/or anti-CD20 activity that is imparted to an anti-CD20 antibody comprising the optimized CDR. “Anti-CD20 activity” can include, e.g., activity which modulates one or more of the following activities associated with CD20, e.g., the ability to induce apoptosis of B-cells, the ability to induce ADCC against B-cells (e.g., CLL cells), the ability to inhibit NF-kappaB activity, the ability to inhibit Snail expression, the ability to de-repress RKIP, the ability to de-repress PTEN, the ability to sensitize a tumor cell to TRAIL-apoptosis or any other activity associated with CD20. Such activities are described, for example, in Baritaki, S. et al., Int. J. Oncol. 38: 1683-1694 (2011), which is herein incorporated by reference in its entirety. The modifications can involve replacement of amino acid residues within the CDR such that an anti-CD20 antibody retains specificity for the CD20 antigen and has improved binding affinity and/or improved anti-CD20 activity.

In certain anti-CD20 antibodies, or antigen-binding fragments thereof, at least a fraction of one or more of the constant region domains has been deleted or otherwise altered so as to provide desired biochemical characteristics such as reduced effector functions, the ability to non-covalently dimerize, increased ability to localize at the site of a tumor, reduced serum half-life, or increased serum half-life when compared with a whole, unaltered antibody of approximately the same immunogenicity. For example, certain antibodies are domain deleted antibodies which comprise a polypeptide chain similar to an immunoglobulin heavy chain, but which lack at least a portion of one or more heavy chain domains. For instance, in certain antibodies, one entire domain of the constant region of the modified antibody will be deleted, for example, all or part of the CH2 domain will be deleted.

In certain anti-CD20 antibodies or antigen-binding fragments thereof, the Fc portion can be mutated to decrease effector function using techniques known in the art. For example, modifications of the constant region can be used to modify disulfide linkages or oligosaccharide moieties that allow for enhanced localization due to increased antigen specificity or antibody flexibility. The resulting physiological profile, bioavailability and other biochemical effects of the modifications can easily be measured and quantified using well know immunological techniques without undue experimentation.

In certain embodiments, an anti-CD20 antibody or antigen-binding fragment thereof will not elicit a deleterious immune response in the animal to be treated, e.g., in a human. In one embodiment, anti-CD20 antibodies or antigen-binding fragments thereof can be modified to reduce their immunogenicity using art-recognized techniques. For example, antibodies can be humanized, primatized, deimmunized, or chimeric antibodies can be made. These types of antibodies are derived from a non-human antibody, typically a murine or primate antibody, that retains or substantially retains the antigen-binding properties of the parent antibody, but which is less immunogenic in humans. This can be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison, S. L. et al., Proc. Natl. Acad. Sci. 81:6851-6855 (1984); Morrison, S. L. et al., Adv. Immunol. 44:65-92 (1988); Verhoeyen, M. et al., Science 239:1534-1536 (1988); Padlan, E.A., Molec. Immun. 28:489-498 (1991); Padlan, E. A., Molec. Immun. 31:169-217 (1994), and U.S. Pat. Nos. 5,585,089, 5,693,761, 5,693,762, and 6,190,370, all of which are hereby incorporated by reference in their entirety.

Modified forms of antibodies or antigen-binding fragments thereof can be made from whole precursor or parent antibodies using techniques known in the art.

Anti-CD20 antibodies or antigen-binding fragments thereof can be made or manufactured using techniques that are known in the art. In certain embodiments, antibody molecules or fragments thereof are “recombinantly produced,” i.e., are produced using recombinant DNA technology. Anti-CD20 antibodies or fragments thereof can be generated by any suitable method known in the art including generation of polyclonal antibodies or preparation of monoclonal antibodies, e.g., through hybridoma or phage display.

A variety of host-expression vector systems can be utilized to express antibody molecules. The host cell can be co-transfected with two expression vectors, the first vector encoding a heavy chain derived polypeptide and the second vector encoding a light chain derived polypeptide. The two vectors can contain identical selectable markers which enable equal expression of heavy and light chain polypeptides. Alternatively, a single vector can be used which encodes both heavy and light chain polypeptides. In such situations, the light chain is advantageously placed before the heavy chain to avoid an excess of toxic free heavy chain (Proudfoot, Nature 322:52 (1986); Kohler, PNAS 77:2197 (1980)). The host cell can also be transfected with a single vector encoding a heavy chain derived polypeptide and a light chain derived polypeptide. The coding sequences for the heavy and light chains can comprise cDNA or genomic DNA.

The expression vector or vectors can be transferred to a host cell by conventional techniques and the transfected cells are then cultured by conventional techniques to produce an antibody. Thus, host cells containing a polynucleotide encoding an antibody, or a heavy or light chain thereof, operably linked to a heterologous promoter are provided. In certain embodiments for the expression of double-chained antibodies, vectors encoding both the heavy and light chains can be co-expressed in the host cell for expression of the entire immunoglobulin molecule.

Host-expression systems represent vehicles by which the coding sequences of interest can be produced and subsequently purified, but also represent cells which can, when transformed or transfected with the appropriate nucleotide coding sequences, express a CD20 antibody in situ. These include but are not limited to microorganisms such as bacteria (e.g., E. coli, B. subtilis) transformed with recombinant bacteriophage DNA, plasmid DNA, or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BLK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Bacterial cells such as E. coli, or eukaryotic cells, e.g., for the expression of whole recombinant antibody molecules, are used for the expression of a recombinant antibody molecule. For example, mammalian cells such as Chinese hamster ovary (CHO) cells, in conjunction with a vector such as the major intermediate early gene promoter element from human cytomegalovirus, is an effective expression system for antibodies (Cockett et al., BioTechnology 8:2 (1990)). In some embodiments, the anti-CD20 antibody is produced in a host cell that is not a CHO cell.

Once an antibody has been recombinantly expressed, it can be purified by any method known in the art for purifying an immunoglobulin molecule, e.g., by chromatography (ion exchange, affinity, particularly by affinity for the specific antigen after Protein A, and sizing column chromatography), centrifugation, differential solubility, or by any other standard technique for the purification of proteins.

In some embodiments, the anti-CD20 antibody is produced by a rat hybridoma cell line, such as, e.g., YB2/0 (ATCC CRL-1662).

IV. Anti-PD-1 or Anti-PD-L1 Antibodies
A. Anti-PD-1 Antibodies

The present disclosure provides an innovative combination treatment and treatment regimen for patients with hematological cancers. The combination treatment includes, inter alia, administering to a subject in need thereof a therapeutically effective amount of at least one anti-PD1 antibody (e.g., pembrolizumab).

The programmed death receptor-1 (PD-1) is a cell-surface receptor that is expressed on antigen-stimulated T cells, as well as B cells, monocytes, and NKT cells. In normal tissues, PD-1 on T cells acts as part of an immunoregulatory receptor-ligand system that enables self-tolerance by T cells, preventing autoimmunity and an excessive immune response, which could injure normal tissues. When PD-1 is not bound by its ligands, PD-L1 and PD-L2 (broadly expressed on hematopoietic and parenchymal cells), T cells respond to T cell receptor-specific signaling with a normal immune response. However, binding of PD-1 by PD-L1 or PD-L2 suppresses the immune response by inhibiting T cell proliferation, cytokine release, and cytotoxicity (see e.g., Brusa, D. et al., Haematologica. 98:953-963 (2013)). The PD-1 receptor-ligand pathway is used by tumors to evade immune surveillance by inactivating tumor antigen reactive cytotoxic T cells via PD-L1 and/or PD-L2 expression by tumor cells. When tumors “hijack” the PD-1 receptor-ligand pathway, neoplastic cells are allowed to proliferate.

Anti-PD-1 antibodies suitable for the methods (and kits) described herein include those that bind to PD-1 with high specificity and affinity, block the binding of PD-L1 and or PD-L2, and/or inhibit the immunosuppressive effect of the PD-1 signaling pathway.

In any of the embodiments disclosed herein, an anti-PD-1 antibody includes an antigen-binding portion or fragment that binds to the PD-1 receptor and exhibits the functional properties similar to those of whole antibodies in inhibiting ligand binding and upregulating the immune system. In certain embodiments, the anti-PD-1 antibody or antigen-binding portion thereof is a chimeric, humanized, or human monoclonal antibody, or a portion thereof. In certain embodiments, the antibody is a humanized antibody. In other embodiments, the antibody is a human antibody. Antibodies of an IgG1, IgG2, IgG3, or IgG4 isotype can be used.

In some embodiments, the anti-PD-1 antibody or antigen-binding portion thereof comprises a heavy chain constant region that is of a human IgG1 or IgG4 isotype. In some embodiments, the antibody comprises a light chain constant region that is a human kappa or lambda constant region. In other embodiments, the anti-PD-1 antibody or antigen-binding portion thereof is a monoclonal antibody or an antigen-binding portion thereof.

In some embodiments, the anti-PD-1 antibody used in the methods (and kits) described herein is nivolumab, pembrolizumab, or pidilizumab.

In some embodiments, the anti-PD-1 antibody is Nivolumab (trade name OPDIVO®; formerly designated 5C4, BMS-936558, MDX-1106, or ONO-4538). Nivolumab is a fully humanized IgG4 (S228P) PD-1 antibody that selectively prevents interaction with PD-1 ligands (PD-L1 and PD-L2), thereby blocking the down-regulation of antitumor T-cell functions (U.S. Pat. No. 8,008,449; WO2006/121168; Wang et al., Cancer Immunol Res. 2:846-56 (2014); Topalian, S. L. et al., N Engl J Med 366:2443-2454 (2012); Topalian, S. L. et al., Current Opinion in Immunology 24:207-212 (2012); Topalian, S. L. et al., J Clin Oncol 31 (suppl):3002 (2013)). Nivolumab has been approved by the U.S. FDA for the treatment of patients with unresectable or metastatic melanoma, metastatic squamous non-small cell lung cancer, advanced renal cell carcinoma, and classical Hodgkin lymphoma.

Pembrolizumab (trade name KEYTRUDA®; also known as lambrolizumab and MK-3475) is a humanized monoclonal IgG4 kappa antibody directed against PD-1. Hamid, O. et al., N Engl J Med 369:134-144 (2013). Pembrolizumab is described, for example, in U.S. Pat. Nos. 8,354,509 and 8,900,587 and WO2009/114335. Pembrolizumab has been approved by the U.S. FDA for the treatment of patients with advanced melanoma, non-small cell lung cancer, and head and neck squamous cell cancer. See, e.g., Poole, R. M., Drugs 74:1973-1981 (2014). In a preferred embodiment, the anti-PD-1 antibody used in the methods (and kits) described herein is pembrolizumab.

Pidilizumab (also known as CT-011 and MDV9300) is a humanized IgG1 kappa monoclonal antibody that binds to PD-1. Pidilizumab is in development by Medivation for the treatment of cancer and infectious diseases. Pidilizumab is described, for example, in U.S. Pat. No. 8,686,119 B2, WO 2013/014668 Al, WO2009/101611, Berger, R. et al., Clinical Cancer Research 14:3044-3051 (2008), and Armand, P. et al., J Clin Oncol 31:4199-4206 (2013).

While not wishing to be bound by theories, it is believed that hematological cancers, such as CLL, utilize immune dysregulation to evade cell death and promote tumor survival. Preclinical data demonstrates the importance PD-1 signaling in both the CLL clone (B regulatory immunophenotype) and the T cell repertoire in CLL. See Ringelstein-Harlev, S. et al., Blood 124:3319 (2014). Thus, anti-PD-1 antibodies (e.g., pembrolizumab) can therapeutically target both the CLL clone directly as well as correct defects in host T cell function that may allow CLL to escape immune surveillance.

Recent data from two studies demonstrated the activity and potential of both pembrolizumab and nivolumab in treating heavily pretreated patients with Hodgkin lymphoma and B cell lymphoproliferative disorders. See Moskowitz, C. H. et al., “PD-1 blockade with the monoclonal antibody pembrolizumab (MK-3475) in patients with classical Hodgkin lymphoma after brentuximab vedotin failure: preliminary results from a phase 1b study (KEYNOTE-013),” Blood 124:290 (2014)) and Lesokhin, A. M. et al., “Preliminary Results of a Phase I Study of Nivolumab (BMS-936558) in Patients with Relapsed or Refractory Lymphoid Malignancies,” Blood 124:291 (2014).

In some embodiments, in the methods (and kits) described herein, pembrolizumab is administered at a dosage range from about 100 to about 300 mg, about 100 to about 200 mg, about 100 mg, about 150 mg, about 200 mg, about 250 mg or about 3000 mg about once every 2 to 4 weeks, or about once every 3 to 4 weeks, about once every 2 weeks, about once every 3 weeks, or about once every 4 weeks. One skilled in the art will appreciate that the dosage of pembrolizumab and/or frequency of administering pembrolizumab may change during the course of therapy (lowered or increased) depending upon a patient's clinical response, side effects, etc., or during different phases of therapy (induction, treatment, or maintenance).

In some embodiments, the pembrolizumab is formulated and/or administered intravenously, preferably by infusion.

Other anti-PD-1 antibodies can be used in the methods and kits described herein, such as, e.g., AMP 514 (Amplimmune), PDR-001 (Novartis), MEDI-0690 (also known as AMP-514) (MedImmune LLC), SHR-1210 (Incyte Corp.), REGN-2810 (Regeneron Pharmaceuticals, Inc.), PF-06801591 (Pfizer), TSR-042 (also known as ANB011)(Tesaro, Inc.), BGB-A317 (BeiGene, Ltd.), and JS001 (Shanghai Junshi Bioscience Co., Ltd.).

In some embodiments, the anti-PD-1 antibody is AMP-224 (Amplimmune; also known as B7-DCIg). AMP-224 is disclosed, e.g., in WO2010/027827 and WO2011/066342. AMP-224 is a PD-L2 Fc fusion protein that blocks the interaction between PD-1 and B7-H1.

Other anti-PD-1 antibodies that can be used in the methods and kits of the invention are described in U.S. Pat. No. 8,609,089, U.S. Patent Publ. No. 2010/028330, and/or U.S. Patent Publ. No. 2012/0114649.
B. Anti-PD-L1 Antibodies

The present disclosure provides an innovative combination treatment and treatment regimen for patients with hematological malignancies. The combination treatment includes, inter alia, administering to a subject in need thereof a therapeutically effective amount of at least one anti-PD-L1 antibody. PD-L1 is the principal ligand of the PD-1 receptor. Because anti-PD-1 and anti-PD-L1 antibodies target the same signaling pathway and have been shown in clinical trials to exhibit similar levels of efficacy in a variety of cancers, including renal cell cancer (RCC) (see Brahmer, J. R. et al., N Engl J Med 366:2455-2465 (2012); Topalian, S. L. et al., N Engl J Med 366:2443-2454 (2012a); WO 2013/173223), an anti-PD-L1 antibody can be used instead of an anti-PD-1 antibody in any of the methods (and kits) disclosed herein.

PD-L1 (formerly B7-H1) is a B7 family member that is expressed on many cell types, including antigen-presenting cells (“APCs”) and activated T cells (Yamazaki, T. et al., J Immunol. 169:5538-5545 (2002)). PD-L1 binds to both PD-1 (CD279) and B7-1. Both the binding of T-cell-expressed B7-1 by PD-L1 and the binding of T-cell-expressed PD-L1 by B7-1 result in T cell inhibition (Butte, M. J. et al., Immunity 27: 111-122 (2007)). There is also evidence that, like other B7 family members, PD-L1 can also provide costimulatory signals to T cells (Subudhi, S. K. et al., J Clin. Invest. 113:694-700 (2004); Tamura, H. et al., Blood 97:1809-1816 (2001)). Furthermore, expression of PD-L1 on the cell surface has also been shown to be upregulated through IFN-γ stimulation.

The interaction between PD-1 and its ligand partners PD-L1 (B7-H1; CD274) and PD-L2 (B7-DC; CD273) is an important negative costimulatory signaling pathway involved in the regulation of T cell activation. PD-1 can be expressed on T cells, B cells, natural killer T (NKT) cells, activated monocytes and dendritic cells (DCs). PD-1 is expressed by activated, but not by unstimulated, human CD4⁺ and CD8⁺ T cells, B cells and myeloid cells. Nishimura, H. et al., Int. Immunol. 8: 773-780 (1996); Boettler, T. et al., J Virol. 80: 3532-3540 (2006). There are at least 4 variants of PD-1 that have been cloned from activated human T cells, including transcripts lacking (i) exon 2, (ii) exon 3, (iii) exons 2 and 3 or (iv) exons 2 through 4. Nielsen, C. et al., Cell. Immunol. 235: 109-116 (2005).

Normal human tissues seldom express PD-L1 protein on their cell surface, with the exception of tonsil, placenta, and a small fraction of macrophage-like cells in lung and liver, suggesting that under normal physiological conditions, PD-L1 mRNA is under tight posttranscriptional regulation. In contrast, PD-L1 protein is abundantly expressed on the cell surface in various human cancers. Chen, L. and Han, X., J. Clin. Invest. 125: 3384-3391 (2015).

PD-L2 expression, on the other hand, is more restricted than PD-L1. For instance, PD-L2 is inducibly expressed on DCs, macrophages, and bone marrow-derived mast cells.

Additionally, several studies show a receptor for PD-L1 that is independent of PD-1. B7.1 has also been identified as a binding partner for PD-L1. Butte, M. J. et al., Immunity 27: 111-122 (2007). Chemical crosslinking studies suggest that PD-L1 and B7.1 can interact through their IgV-like domains. B7.1:PD-L1 interactions can induce an inhibitory signal into T cells. Ligation of PD-L1 on CD4⁺ T cells by B7.1 or ligation of B7.1 on CD4⁺ T cells by PD-L1 delivers an inhibitory signal. T cells lacking CD28 and CTLA-4 show decreased proliferation and cytokine production when stimulated by anti-CD3 plus B7.1 coated beads. In T cells lacking all the receptors for B7.1 (i.e., CD28, CTLA-4 and PD-L1), T cell proliferation and cytokine production were no longer inhibited by anti-CD3 plus B7.1 coated beads. This indicates that B7.1 acts specifically through PD-L1 on the T-cell in the absence of CD28 and CTLA-4. Similarly, T cells lacking PD-1 showed decreased proliferation and cytokine production when stimulated in the presence of anti-CD3 plus PD-L1 coated beads, demonstrating the inhibitory effect of PD-L1 ligation on B7.1 on T cells. When T cells lack all known receptors for PD-L1 (i.e., no PD-1 and B7.1), T cell proliferation was no longer impaired by anti-CD3 plus PD-L1 coated beads. Thus, PD-L1 can exert an inhibitory effect on T cells either through B7.1 or PD-1.

PD-L1 expression has been found in several murine and human cancers, including human lung, ovarian, and colon carcinoma, and various myelomas (Iwai, Y. et al., PNAS 99: 12293-12297 (2002); Ohigashi, Y. et al., Clin Cancer Res 11:2947-2953 (2005)). PD-L1 has been suggested to play a role in tumor immunity by increasing apoptosis of antigen-specific T-cell clones (Dong, H. et al., Nat Med 8:793-800 (2002)).

The anti-PD-L1 antibodies and functional fragments thereof that can be used in the methods (and kits) of the present invention will have a variety of functional properties for treating cancers or malignant disease, including, but not limited to, having antibody dependent cellular cytotoxic (ADCC) activity, having antitumor activity, inhibiting the binding of PD-L1 to PD-1, and preventing PD-1 mediated inhibition of T-cell activation.

Further, as a result of the antagonism of signaling through PD-L1, including blocking PD-L1 from interacting with either PD-1, B7.1, or both, the disclosed antibodies and functional fragments will prevent PD-L1 from sending a negative costimulatory signal to T-cells and other antigen presenting cells, thus enhancing anti-tumor immunity and the immunological defense against cancer and malignant disease.

The PD-L1 antibodies can be polyclonal, monoclonal, chimeric, human, partially or fully humanized, and/or recombinant. For example, in some embodiments, the anti-PD-L1 antibody is a polyclonal antibody or a PD-L1-binding functional fragment thereof. In some embodiments, the anti-PD-L1 antibody is a monoclonal antibody or a PD-L1-binding functional fragment thereof. In some embodiments, the antibodies and functional fragments thereof can bind human, cyno, and/or murine PD-L1.

Polyclonal antibodies may be obtained by methods known in the art, such as by immunizing a selected animal with a PD-L1 antigen, collecting serum from the animal, and isolating and/or purifying antibodies from the serum. Monoclonal antibodies (mAbs) may be obtained by methods known in the art, for example, by fusing antibody-producing cells with immortalized cells to obtain a hybridoma, and/or by generating mAbs from mRNA extracted from bone marrow and spleen cells of immunized animals using combinatorial antibody library technology. Recombinant antibodies may be obtained by methods known in the art, for example, using phage or yeast display technologies and/or expressing or co-expressing antibody polypeptides. Other techniques for making antibodies are known in the art, and can be used to obtain antibodies used in the methods described herein.

The terms “PD-L1-binding functional fragment” or “functional fragment,” as used herein, refer to one or more fragments of an anti-PD-L1 antibody that retain the ability to bind PD-L1. Examples of binding fragments include (i) Fab fragments (monovalent fragments consisting of the VL, VH, CL, and CH1 domains); (ii) F(ab′)2 fragments (bivalent fragment comprising two Fab fragments linked by a disulfide bridge at the hinge region); (iii) Fd fragments (comprising the VH and CH1 domains); (iv) Fv fragments (comprising the VL and VH domains of a single arm of an antibody), (v) dAb fragments (comprising a VH domain); and (vi) isolated complementarity determining regions (CDR), e.g., VH CDR3. Other examples include single chain Fv (scFv) constructs. See e.g., Bird, R. E. et al., Science 242:423-426 (1988); Huston, J. S. et al., Proc. Natl. Acad. Sci. USA 85:5879-5883 (1988). Other examples include PD-L1-binding-domain immunoglobulin fusion proteins comprising (i) a PD-L1-binding domain polypeptide (such as a heavy chain variable region, a light chain variable region, or a heavy chain variable region fused to a light chain variable region via a linker peptide) fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region.

The hinge region of the disclosed antibodies may be modified by replacing one or more cysteine residues with, for example, serine residues, to prevent dimerization. See e.g., U.S. Patent Appl. Publ. No. 2003/0118592; U.S. Patent Appl. Publ. No. U.S. 2003/0133939. Additionally, in some embodiments, the disclosed antibodies may comprise other mutations, including but not limited to a variant Fc portion of an IgG1 having the point mutations S239D/1332E, S239D, or 1332E, or any combination thereof, or a variant Fe portion of an IgG4 having the point mutation S228P. Such modifications may alter the binding of the disclosed antibodies and functional fragments to Fe receptors (FcRs), and in some embodiments, the antibody may be modified to be more stable, while in some embodiments, the antibody may be modified to enhance ADCC function. When determining the number of the residue, the Kabat numbering of residues may be determined for a given antibody by alignment at regions of homology of the sequence of the antibody with a “standard” Kabat numbered sequence.

In some embodiments, the glycosylation patterns of the anti-PD-L1 antibodies may be modified or altered. For instance, in some embodiments, the disclosed antibodies and functional fragments thereof may be low fucose antibodies or they may be defucosylated or the antibodies may be expressed or produced in such a way that they are lacking fucose altogether (i.e., afucosylated). Modifying the fucose content of the antibody or functional fragment may be accomplished through various means known in the art, for instance, expressing the antibody or functional fragment in a cell that is FUT8 deficient or that has a mutated version of FUT8. Low fucose or defucosylated antibodies and functional fragment have increased ADCC activity. In addition to alterations in fucose, the disclosed antibodies and functional fragments may comprise other functional modifications to their glycosylation patterns. For instance, modifications at position 297 (e.g., N297A and N297Q) can prevent glycosylation of the Fc region altogether, thus eliminating Fe function, ADCC, and CDC.

In some embodiments, the anti-PD-L1 antibody used in the methods and kits of the invention is CTI-07, CTI-09, CTI-48, CTI-49, CTI-50, CTI-76, CTI-77, CTI-78, CTI-57, CTI-58, CTI-92, CTI-93, CTI-94, CTI-95, CTI-96, CTI-97, CTI-98, or a functional fragment thereof. See U.S. patent application Ser. No. 15/636,610 and PCT/US2017/039810, filed Jun. 28, 2017, which are incorporated by reference in their entirety. Tables 4 and 5 provide exemplary heavy chain CDR sequences (HCDR1, HCDR2, and HCDR3) and light chain CDR sequences (LCDR1, LCDR2, and LCDR3) of these anti-PD-L1 antibodies and functional fragments thereof.

In some embodiments, the anti-PD-L1 antibody used in the methods and kits of the invention is CTI-48, which is also known as CK-301. CK-301, which is in development by Checkpoint Therapeutics, Inc. and TG Therapeutics, Inc., is a novel, fully human PD-L1 specific IgGlantibody,which exhibits sub-nanomolar binding affinity for PD-L1. CK-301 blocks binding of PD-L1 to both PD-land B7-1 in enzyme-linked immunosorbent assays (ELISA) and cell-based competition assays. Phase I clinical trials of CK-301 are underway. See, Gorelik, L., et al., “Preclinical characterization of a novel fully human IgG1 anti-PD-L1 mAb CK-301,” American Association for Cancer Research Annual Meeting (AACR), Washington, D.C., Abstract No. 4606 (Apr. 4, 2017).

TABLE 4

| Antibody | HCDR1 | HCDR2 | HCDR3 |
|---|---|---|---|
| CTI-48 | GTFSRSAIS | VIIPAFGEANYAQKFQG | ARGRQMFGAGIDF |
| | (SEQ ID NO: 11) | (SEQ ID NO: 19) | (SEQ ID NO: 27) |
| CTI-49 | GTFSGYAIS | VIIPAFGTANYAQKFQG | ARGRQMFGAGIDF |
| | (SEQ ID NO: 12) | (SEQ ID NO: 20) | (SEQ ID NO: 27) |
| CTI-76 | GTFWRYAIS | VIIPIWGKANYAQKFQG | ARGRQMFGAGIDF |
| | (SEQ ID NO: 13) | (SEQ ID NO: 21) | (SEQ ID NO: 27) |
| CTI-77 | GTFGSYAIS | GIYPAFGTANYAQKFQG | ARGRQMFGAGIDF |
| | (SEQ ID NO: 14) | (SEQ ID NO: 22) | (SEQ ID NO: 27) |
| CTI-78 | GTFGTYAIS | GIYPRFGTANYAQKFQG | ARGRQMFGAGIDF |
| | (SEQ ID NO: 15) | (SEQ ID NO: 23) | (SEQ ID NO: 27) |
| CTI-50 | GTFSPKAIS | VIIPIFGPANYAQKFQG | ARGRQMFGAGIDF |
| | (SEQ ID NO: 16) | (SEQ ID NO: 24) | (SEQ ID NO: 27) |
| CTI-57 | YTLSSHGIT | WISAHSGHASNAQKVED | ARVWRALYHGMDV |
| | (SEQ ID NO: 17) | (SEQ ID NO: 25) | (SEQ ID NO: 28) |
| CTI-58 | YTLSSHGIT | WISAHSGHASNAQKVED | ARVHAALYHGMDV |
| | (SEQ ID NO: 17) | (SEQ ID NO: 25) | (SEQ ID NO: 29) |
| CTI-09 | GTFSSYAIS | GIIPIFGTANYAQKFQG | ARGRQMFGAGIDF |
| | (SEQ ID NO: 18) | (SEQ ID NO: 26) | (SEQ ID NO: 27) |
| CTI-07 | YTLSSHGIT | WISAHSGHASNAQKVED | ARVHAALYYGMDV |
| | (SEQ ID NO: 17) | (SEQ ID NO: 25) | (SEQ ID NO: 30) |
| CTI-97 | GTFSRSAIS | VIIPAFGEANYAQKFQG | ARGRQMFGAGIDF |
| | (SEQ ID NO: 11) | (SEQ ID NO: 19) | (SEQ ID NO: 27) |
| CTI-98 | GTFSRSAIS | VIIPAFGEANYAQKFQG | ARGRQMFGAGIDF |
| | (SEQ ID NO: 11) | (SEQ ID NO: 19) | (SEQ ID NO: 27) |
| CTI-92 | GTFSRSAIS | VIIPAFGEANYAQKFQG | ARGRQMFGAGIDF |
| | (SEQ ID NO: 11) | (SEQ ID NO: 19) | (SEQ ID NO: 27) |
| CTI-95 | GTFSRSAIS | VIIPAFGEANYAQKFQG | ARGRQMFGAGIDF |
| | (SEQ ID NO: 11) | (SEQ ID NO: 19) | (SEQ ID NO: 27) |
| CTI-93 | GTFSRSAIS | VIIPAFGEANYAQKFQG | ARGRQMFGAGIDF |
| | (SEQ ID NO: 11) | (SEQ ID NO: 19) | (SEQ ID NO: 27) |
| CTI-94 | GTFSRSAIS | VIIPAFGEANYAQKFQG | ARGRQMFGAGIDF |
| | (SEQ ID NO: 11) | (SEQ ID NO: 19) | (SEQ ID NO: 27) |
| CTI-96 | GTFSRSAIS | VIIPAFGEANYAQKFQG | ARGRQMFGAGIDF |
| | (SEQ ID NO: 11) | (SEQ ID NO: 19) | (SEQ ID NO: 27) |

TABLE 5

| Antibody | LCDR1 | LCDR2 | LCDR3 |
|---|---|---|---|
| CTI-48 | TRSSGSIDSNYVQ | EDNQRPS | QSYDSNNRHVI |
| | (SEQ ID NO: 31) | (SEQ ID NO: 33) | (SEQ ID NO: 35) |
| CTI-49 | TRSSGSIDSNYVQ | EDNQRPS | QSYDSNNRHVI |
| | (SEQ ID NO: 31) | (SEQ ID NO: 33) | (SEQ ID NO: 35) |
| CTI-76 | TRSSGSIDSNYVQ | EDNQRPS | QSYDSNNRHVI |
| | (SEQ ID NO: 31) | (SEQ ID NO: 33) | (SEQ ID NO: 35) |
| CTI-77 | TRSSGSIDSNYVQ | EDNQRPS | QSYDSNNRHVI |
| | (SEQ ID NO: 31) | (SEQ ID NO: 33) | (SEQ ID NO: 35) |
| CTI-78 | TRSSGSIDSNYVQ | EDNQRPS | QSYDSNNRHVI |
| | (SEQ ID NO: 31) | (SEQ ID NO: 33) | (SEQ ID NO: 35) |
| CTI-50 | TRSSGSIDSNYVQ | EDNQRPS | QSYDSNNRHVI |
| | (SEQ ID NO: 31) | (SEQ ID NO: 33) | (SEQ ID NO: 35) |
| CTI-57 | GGNNIGSKGVH | DDSDRPS | QVWDSSSDHWV |
| | (SEQ ID NO: 32) | (SEQ ID NO: 34) | (SEQ ID NO: 36) |
| CTI-58 | GGNNIGSKGVH | DDSDRPS | QVWDSSSDHWV |
| | (SEQ ID NO: 32) | (SEQ ID NO: 34) | (SEQ ID NO: 36) |
| CTI-09 | TRSSGSIDSNYVQ | EDNQRPS | QSYDSNNRHVI |
| | (SEQ ID NO: 31) | (SEQ ID NO: 33) | (SEQ ID NO: 35) |
| CTI-07 | GGNNIGSKGVH | DDSDRPS | QVWDSSSDHWV |
| | (SEQ ID NO: 32) | (SEQ ID NO: 34) | (SEQ ID NO: 36) |
| CTI-97 | TRSSGSIDSNYVQ | EDNQRPS | QSYDSNLRHVI |
| | (SEQ ID NO: 31) | (SEQ ID NO: 33) | (SEQ ID NO: 85) |
| CTI-98 | TRSSGSIDSNYVQ | EDNQRPS | QSYDSNLRHVI |
| | (SEQ ID NO: 31) | (SEQ ID NO: 33) | (SEQ ID NO: 85) |
| CTI-92 | TRSSGSIDSNYVQ | EDNQRPS | QSYDSNNRHVI |
| | (SEQ ID NO: 31) | (SEQ ID NO: 33) | (SEQ ID NO: 35) |
| CTI-95 | TRSSGSIDSNYVQ | EDNQRPS | QSYDSNNRHVI |
| | (SEQ ID NO: 31) | (SEQ ID NO: 33) | (SEQ ID NO: 35) |
| CTI-93 | TRSSGSIDSNYVQ | EDNQRPS | QSYDSNIRHVI |
| | (SEQ ID NO: 31) | (SEQ ID NO: 33) | (SEQ ID NO: 86) |
| CTI-94 | TRSSGSIDSNYVQ | EDNQRPS | QSYDSNLRHVI |
| | (SEQ ID NO: 31) | (SEQ ID NO: 33) | (SEQ ID NO: 85) |
| CTI-96 | TRSSGSIDSNYVQ | EDNQRPS | QSYDSNIRHVI |
| | (SEQ ID NO: 31) | (SEQ ID NO: 33) | (SEQ ID NO: 86) |

Additionally, the disclosed anti-PD-L1 antibodies and functional fragments may also comprise various framework regions. For instance, in some embodiments, the disclosed antibodies and functional fragments comprise SEQ ID NOs: 37-45 and/or 54-58.

In some embodiments, the variable heavy framework region 1 comprises SEQ ID NO: 37 or SEQ ID NO: 38. In some embodiments, the variable heavy framework region 2 comprises SEQ ID NO: 39, the variable heavy framework region 3 comprises SEQ ID NO: 40, and/or the variable heavy framework region 4 comprises SEQ ID NO: 41.

In some embodiments, the variable light framework region 1 comprises SEQ ID NO: 42, the variable light framework region 2 comprises SEQ ID NO: 43, the variable light framework region 3 comprises SEQ ID NO: 44, SEQ ID NO:87, or SEQ ID NO:88, and/or the variable light framework region 4 comprises SEQ ID NO: 45.

In some embodiments, the variable heavy chain sequence comprises an amino acid sequence selected from the group consisting of SEQ ID NOS: 47, 48, 49, 50, 51, and 52. In other embodiments, the variable heavy chain sequence comprises an amino acid sequence selected from the group consisting of SEQ ID NOS: 62, 63, 64, 65, 66, and 67.

In some embodiments, the variable light chain sequence comprises SEQ ID NO:53.

In some embodiments, the anti-PD-L1 antibody or functional fragment thereof comprises a heavy chain comprising SEQ ID NO: 46.

In some embodiments, the anti-PD-L1 antibody or functional fragment thereof comprises a heavy chain comprising: HCDR1 comprising SEQ ID NO: 17; HCDR2 comprising SEQ ID NO: 25; and HCDR3 comprising amino acids 6-13 of SEQ ID NO: 28; and a light chain comprising: LCDR1 comprising SEQ ID NO: 32; LCDR2 comprising SEQ ID NO: 34; and LCDR3 comprising SEQ ID NO: 36.

In some embodiments, the HCDR3 comprises SEQ ID NO: 28, and in some embodiments, the CDRH3 comprises SEQ ID NO: 29.

In some embodiments, the variable heavy framework region 1 comprises SEQ ID NO: 60, the variable heavy framework region 2 comprises SEQ ID NO: 39, the variable heavy framework region 3 comprises SEQ ID NO: 61, and/or the variable heavy framework region 4 comprises SEQ ID NO: 41.

In some embodiments, the variable light framework region 1 comprises SEQ ID NO: 56, the variable light framework region 2 comprises SEQ ID NO: 57, the variable light framework region 3 comprises SEQ ID NO: 58, and/or the variable light framework region 4 comprises SEQ ID NO: 45.

In some embodiments, the variable heavy chain sequence comprises SEQ ID NO: 59 or 60, and in some embodiments, the variable light chain sequence comprises SEQ ID NO: 61.

In some embodiments, certain alterations to the framework regions may be particularly advantageous. For instance, substituting glutamic acid (E) for glutamine (Q) in the first position of framework region one of the heavy chain of an antibody can increase manufacturing product stability efficiency. Accordingly, some embodiments of the disclosed antibodies and fragments will incorporate this modification. Thus, in some embodiments, the heavy chain of the disclosed antibodies or functional fragments will comprise SEQ ID NOs:47-52, while in other embodiments, the heavy chain of the disclosed antibodies or functional fragments will comprise SEQ ID NOs: 59-60 or 62-67. Furthermore, in some embodiments, the heavy chain of the disclosed antibodies or functional fragments will comprise SEQ ID NOs: 81-82, or a polypeptide encoded by a nucleic acid sequence comprising SEQ ID NOs: 69-78.

In some embodiments, the light chain of the disclosed antibodies or functional fragments will comprise SEQ ID NOs: 53 or 61. Furthermore, in some embodiments, the light chain of the disclosed antibodies or functional fragments will comprise a polypeptide encoded by a nucleic acid sequence comprising SEQ ID NOs:79-80.

One of ordinary skill in the art will understand that certain changes can be made to the disclosed sequences without compromising the binding affinity or function of the disclosed anti-PD-L1 antibodies and functional fragments. According, in some embodiments, the anti-PD-L1 antibodies or functional fragments will share about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the disclosed sequences.

In some embodiments, the disclosure provides for isolated nucleic acid sequences encoding an anti-PD-L1 antibody or functional fragment thereof, for example, SEQ ID NOs:69-80.

The disclosed antibodies and functional fragments thereof may be defined by sequence, or by functional characteristics. For instance, the disclosed antibodies and functional fragments thereof, can have a K_{D} of at least 3.0×10⁻⁸, at least 2.5×10⁻⁸, at least 2.0×10⁻⁸, at least 1.5×10⁻⁸, at least 1.0×10⁻⁸, at least 0.5×10⁻⁸, at least 9.95×10⁻⁹, at least 9.90×10⁻⁹, at least 9.85×10⁻⁹, at least 9.80×10⁻⁹, at least 9.75×10⁻⁹, at least 9.70×10⁻⁹, at least 9.65×10⁻⁹, at least 9.60×10⁻⁹, at least 9.55×10⁻⁹, at least 9.5×10⁻⁹, at least 9.45×10⁻⁹, at least 9.40×10⁻⁹, at least 9.35×10⁻⁹, at least 9.30×10⁻⁹, at least 9.25×10⁻⁹, at least 9.20×10⁻⁹, at least 9.15×10⁻⁹, at least 9.10×10⁻⁹, at least 9.05×10⁻⁹, at least 9.0×10⁻⁹, at least 8.95×10⁻⁹, at least 8.90×10⁻⁹, at least 8.85×10⁻⁹, at least 8.80×10⁻⁹, at least 8.75×10⁻⁹, at least 8.70×10⁻⁹, at least 8.65×10⁻⁹, at least 8.60×10^{-9,} at least 8.55×10⁻⁹, at least 8.5×10⁻⁹, at least 8.45×10⁻⁹, at least 8.40×10⁻⁹, at least 8.35×10⁻⁹, at least 8.30×10⁻⁹, at least 8.25×10⁻⁹, at least 8.20×10⁻⁹, at least 8.15×10⁻⁹, at least 8.10×10⁻⁹, at least 8.05×10⁻⁹, at least 8.0×10⁻⁹, at least 7.95×10⁻⁹, at least 7.90×10⁻⁹, at least 7.85×10⁻⁹, at least 7.80×10⁻⁹, at least 7.75×10⁻⁹, at least 7.70×10⁻⁹, at least 7.65×10⁻⁹, at least 7.60×10⁻⁹, at least 7.55×10⁻⁹, at least 7.5×10⁻⁹, at least 7.45×10⁻⁹, at least 7.40×10⁻⁹, at least 7.35×10⁻⁹, at least 7.30×10⁻⁹, at least 7.25×10⁻⁹, at least 7.20×10⁻⁹, at least 7.15×10⁻⁹, at least 7.10×10⁻⁹, at least 7.05×10⁻⁹, at least 7.0×10⁻⁹, at least 6.95×10⁻⁹, at least 6.90×10⁻⁹, at least 6.85×10⁻⁹, at least 6.80×10⁻⁹, at least 6.75×10⁻⁹, at least 6.70×10⁻⁹, at least 6.65×10⁻⁹, at least 6.60×10⁻⁹, at least 6.55×10⁻⁹, at least 6.5×10⁻⁹, at least 6.45×10⁻⁹, at least 6.40×10⁻⁹, at least 6.35×10⁻⁹, at least 6.30×10⁻⁹, at least 6.25×10⁻⁹, at least 6.20×10⁻⁹, at least 6.15×10⁻⁹, at least 6.10×10⁻⁹, at least 6.05×10^{-9,} at least 6.0×10⁻⁹, at least 5.95×10⁻⁹, at least 5.90×10⁻⁹, at least 5.85×10⁻⁹, at least 5.80×10⁻⁹, at least 5.75×10⁻⁹, at least 5.70×10⁻⁹, at least 5.65×10⁻⁹, at least 5.60×10⁻⁹, at least 5.55×10⁻⁹, at least 5.5×10⁻⁹, at least 5.45×10⁻⁹, at least 5.40×10⁻⁹, at least 5.35×10⁻⁹, at least 5.30×10⁻⁹, at least 5.25×10⁻⁹, at least 5.20×10⁻⁹, at least 5.15×10⁻⁹, at least 5.10×10⁻⁹, at least 5.05×10⁻⁹, at least 5.0×10⁻⁹, at least 4.95×10⁻⁹, at least 4.90×10⁻⁹, at least 4.85×10⁻⁹, at least 4.80×10⁻⁹, at least 4.75×10⁻⁹, at least 4.70×10⁻⁹, at least 4.65×10⁻⁹, at least 4.60×10⁻⁹, at least 4.55×10⁻⁹, at least 4.5×10⁻⁹, at least 4.45×10⁻⁹, at least 4.40×10⁻⁹, at least 4.35×10⁻⁹, at least 4.30×10⁻⁹, at least 4.25×10⁻⁹, at least 4.20×10⁻⁹, at least 4.15×10⁻⁹, at least 4.10×10⁻⁹, at least 4.05×10⁻⁹, at least 4.0×10⁻⁹, at least 3.95×10⁻⁹, at least 3.90×10⁻⁹, at least 3.85×10⁻⁹, at least 3.80×10⁻⁹, at least 3.75×10⁻⁹, at least 3.70×10⁻⁹, at least 3.65×10⁻⁹, at least 3.60×10⁻⁹, at least 3.55×10⁻⁹, at least 3.5×10⁻⁹, at least 3.45×10⁻⁹, at least 3.40×10⁻⁹, at least 3.35×10⁻⁹, at least 3.30×10⁻⁹, at least 3.25×10⁻⁹, at least 3.20×10⁻⁹, at least 3.15×10⁻⁹, at least 3.10×10⁻⁹, at least 3.05×10⁻⁹, at least 3.0×10⁻⁹, at least 2.95×10⁻⁹, at least 2.90×10⁻⁹, at least 2.85×10⁻⁹, at least 2.80×10⁻⁹, at least 2.75×10⁻⁹, at least 2.70×10⁻⁹, at least 2.65×10⁻⁹, at least 2.60×10⁻⁹, at least 2.55×10⁻⁹, at least 2.5×10⁻⁹, at least 2.45×10⁻⁹, at least 2.40×10⁻⁹, at least 2.35×10⁻⁹, at least 2.30×10⁻⁹, at least 2.25×10⁻⁹, at least 2.20×10⁻⁹, at least 2.15×10⁻⁹, at least 2.10×10⁻⁹, at least 2.05×10⁻⁹, at least 2.0×10⁻⁹, at least 1.95×10⁻⁹, at least 1.90×10⁻⁹, at least 1.85×10⁻⁹, at least 1.80×10⁻⁹, at least 1.75×10⁻⁹, at least 1.70×10⁻⁹, at least 1.65×10⁻⁹, at least 1.60×10⁻⁹, at least 1.55×10⁻⁹, at least 1.5×10^{-9,} at least 1.45×10⁻⁹, at least 1.40×10⁻⁹, at least 1.35×10⁻⁹, at least 1.30×10⁻⁹, at least 1.25×10⁻⁹, at least 1.20×10⁻⁹, at least 1.15×10⁻⁹, at least 1.10×10⁻⁹, at least 1.05×10⁻⁹, at least 1.0×10⁻⁹, at least 0.95×10⁻⁹, at least 0.90×10⁻⁹, at least 0.85×10⁻⁹, at least 0.80×10⁻⁹, at least 0.75×10⁻⁹, at least 0.70×10⁻⁹, at least 0.65×10^{-9,} at least 0.60×10⁻⁹, at least 0.55×10⁻⁹, at least 0.5×10⁻⁹, at least 0.45×10⁻⁹, at least 0.40×10⁻⁹, at least 0.35×10⁻⁹, at least 0.30×10⁻⁹, at least 0.25×10⁻⁹, at least 0.20×10⁻⁹, at least 0.15×10⁻⁹, at least 0.10×10⁻⁹, at least 0.05×10⁻⁹, at least 9.5×10⁻¹⁰ at least 9.0×10⁻¹ at least 8.5×10⁻¹ at least 8.0×10⁻¹⁰ or any value in between. For example, the disclosed antibodies and functional fragments thereof can have K_{D} values of 8.2×10⁻¹⁰, 2.3×10⁻⁹, 8.24×10⁻⁹, 3.25×10⁻⁹, 3.46×10⁻⁹, 1.91×10⁻⁹, 7.97×10⁻⁸, 2.41×10⁻⁸, 9.5×−10, or 8.6×10⁻¹⁰.

Likewise, the disclosed antibodies and functional fragments thereof can have IC₅₀ values between 4.0×10⁻⁵ μg/ml and 9.5×10⁻⁷ μg/ml or any value in between. For example, the disclosed antibodies and functional fragments thereof can have IC₅₀ values of 9.19×10⁻⁷, 4.156×10⁻⁵, 9.985×10⁻⁷, 1.037×10⁻⁶, or 3.463×10⁻⁶.

In some embodiments, other anti-PD-L1 antibodies, known to those skilled in the art, can be used in the methods (and kits) described herein. For example, PD-L1 antibodies disclosed in U.S. Patent Publ. No. 2015/0274835, published Oct. 1, 2015, U.S. Patent Publ. No. 2014/0356353, published Dec. 4, 2014, and WO 2010/077634, published Jul. 8, 2010 can be used.

In some embodiments, the anti-PD-L1 antibody used in the methods and kits described herein is durvalumab, BMS-936559, atezolizumab, or avelumab.

In some embodiments, the anti-PD-L1 antibody is Durvalumab (also known as MEDI 4736), a human IgG1 antibody that specifically binds to PD-L1. Durvalumab is in development by AstraZeneca and Medlmmune and is described in, e.g., Lutzky et al., Proc Am Soc Clin Oncol. 35 (Abstract No. 3001)(2014); U.S. Pat. No. 8,779,108; U.S. Patent Appl. Publ. No. 2014/0356353, published Dec. 4, 2014; Khleif, S. et al., Proceedings from the European Cancer Congress 2013; September 27-Oct. 1, 2013, Amsterdam, The Netherlands, Abstract No. 802; Brahmer, J. R. et al., J Clin Oncol 32 (suppl.):5s (Abstract No. 8021)(2014).

In some embodiments, the anti-PD-L1 antibody is BMS-936559 (also known as MDX1105 and 12A4). BMS-936559, developed by Bristol-Myers Squibb, is a fully human IgG4 monoclonal antibody that inhibits binding of PD-L1 to PD-1 and CD80 with high affinity. BMS-936559 is described in, e.g., U.S. Pat. No. 7,943,743, WO2007/005874, and WO 2013/173223.

In some embodiments, the anti-PD-L1 antibody is Atezolizumab (trade name TECENTRIQ® (Genentech/Roche); also known as MPDL3280A and RG7446). Atezolizumab is a fully humanized IgG1 monoclonal antibody that binds to PD-L1. Atezolizumab is described in, e.g., U.S. Pat. Nos. 8,217,149 and 7,943,743 and Herbst, R. S. et al., J Clin Oncol 31(suppl):3000 Abstract (2013). In 2016, TECENTRIQ® received approval by the U.S. FDA for the treatment of bladder cancer.

In some embodiments, the anti-PD-L1 antibody is Avelumab (also known as MSB0010718C), which is in development by Pfizer and Merck. Avelumab is a fully human anti-PD-L1 IgG1 antibody currently being investigated in clinical trials in multiple tumor types. Avelumab is described in U.S. Patent Publ. No. 2014/0341917); Kelly, K. et al., J Clin Oncol 34 (suppl; Abstract No. 3055) (2016); Kaufman. H. et al., J Clin Oncol 34 (suppl; Abstract No. 9508)(2016); Heery, C. R. et al., J Clin Oncol 33 (suppl; Abstract No. 3055)(2015); Heery C. R. et al., J Clin Oncol 33 (suppl; Abstract No. TPS3101)(2015); and Boyerinas, B. et al., Cancer Immunol. Res. 3:1148-1157 (2015).

In some embodiments, the anti-PD-L1 antibody is CX-072 (CytomX Therapeutics), which is a probody targeting PD-L1. In some embodiments, the anti-PD-L1 antibody is GX-P2 (Genexine), which is an anti-PD-L1 fusion protein.

In some embodiments, rather than using an antibody that targets PD-L1, a small molecule that targets PD-L1 can also be used in the methods and kits of the invention. For example CA-170, in development by Curis, Inc., is an orally available small molecule that selectively targets and inhibits PD-L1, PD-L2, and V-domain immunoglobulin suppressor of T-cell activation (VISTA) checkpoint regulators of immune activation. Curis is currently investigating CA-170 in a Phase 1 trial in patients with advanced solid tumors and lymphomas. See www.clinicaltrials.gov (NCT02812875).

V. Pharmaceutical Compositions

The PI3K-delta inhibitors, the anti-CD20 antibodies, and the anti-PD-1 or anti-PD-L1 antibodies used in the methods and kits described herein can be formulated into pharmaceutical compositions suitable for administration. The pharmaceutical compositions may comprise pharmaceutically acceptable excipients. A pharmaceutically acceptable excipient, as used herein, includes, but are not limited to, any and all solvents, dispersion media, or other liquid vehicles, dispersion or suspension aids, diluents, granulating and/or dispersing agents, surface active agents, isotonic agents, thickening or emulsifying agents, preservatives, binders, lubricants or oil, coloring, sweetening or flavoring agents, stabilizers, antioxidants, antimicrobial or antifungal agents, osmolality adjusting agents, pH adjusting agents, buffers, chelants, cyoprotectants, and/or bulking agents, as suited to the particular dosage form desired. Various excipients for formulating pharmaceutical compositions and techniques for preparing the composition are known in the art (see Remington: The Science and Practice of Pharmacy, 21^{st} Ed., A. R. Gennaro (Lippincott, Williams & Wilkins, Baltimore, Md., 2006; incorporated by reference in its entirety)

Exemplary diluents include, but are not limited to, calcium or sodium carbonate, calcium phosphate, calcium hydrogen phosphate, sodium phosphate, lactose, sucrose, cellulose, microcrystalline cellulose, kaolin, mannitol, sorbitol, and/or combinations thereof.

Exemplary granulating and/or dispersing agents include, but are not limited to, starches, pregelatinized starches, or microcrystalline starch, alginic acid, guar gum, agar, poly(vinyl-pyrrolidone), (providone), cross-linked poly(vinyl-pyrrolidone) (crospovidone), cellulose, methylcellulose, carboxymethyl cellulose, cross-linked sodium carboxymethyl cellulose (croscarmellose), magnesium aluminum silicate)(VEEGUM®, sodium lauryl sulfate, and/or combinations thereof.

Exemplary surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), sorbitan fatty acid esters (e.g., polyoxyethylene sorbitan monooleate [TWEEN® 80], sorbitan monopalmitate [SPAN® 40], glyceryl monooleate, polyoxyethylene esters, polyethylene glycol fatty acid esters (e.g.,)CREMOPHOR®), polyoxyethylene ethers (e.g., polyoxyethylene lauryl ether [BRIJ® 30]), PLUORTNC®F 68, POLOXAMER® 188, and/or combinations thereof.

Exemplary binding agents include, but are not limited to, starch, gelatin, sugars (e.g., sucrose, glucose, dextrose, dextrin, molasses, lactose, lactitol, mannitol), amino acids (e.g., glycine), natural and synthetic gums (e.g., acacia, sodium alginate), ethylcellulose, hydroxyethylcellulose, hydroxypropyl methylcellulose, and/or combinations thereof.

Exemplary antioxidants include, but are not limited to, alpha tocopherol, ascorbic acid, acorbyl palmitate, benzyl alcohol, butylated hydroxyanisole, m-cresol, methionine, butylated hydroxytoluene, monothioglycerol, sodium or potassium metabisulfite, propionic acid, propyl gallate, sodium ascorbate, and/or combinations thereof.

Exemplary chelating agents include, but are not limited to, ethylenediaminetetraacetic acid (EDTA), citric acid monohydrate, disodium edetate, fumaric acid, malic acid, phosphoric acid, sodium edetate, tartaric acid, trisodium edetate, and/or combinations thereof.

Exemplary antimicrobial or antifungal agents include, but are not limited to, benzalkonium chloride, benzethonium chloride, methyl paraben, ethyl paraben, propyl paraben, butyl paraben, benzoic acid, hydroxybenzoic acid, potassium or sodium benzoate, potassium or sodium sorbate, sodium propionate, sorbic acid, and/or combinations thereof.

Exemplary preservatives include, but are not limited to, vitamin A, vitamin C, vitamin E, beta-carotene, citric acid, ascorbic acid, butylated hydroxyanisol, ethylenediamine, sodium lauryl sulfate (SLS), sodium lauryl ether sulfate (SLES), and/or combinations thereof.

Exemplary buffers to control pH can include, but are not limited to, sodium phosphate, sodium citrate, sodium succinate, histidine (or histidine-HCl), sodium malate, sodium carbonate, and/or combinations thereof.

Exemplary lubricating agents include, but are not limited to, magnesium stearate, calcium stearate, stearic acid, silica, talc, malt, hydrogenated vegetable oils, polyethylene glycol, sodium benzoate, sodium or magnesium lauryl sulfate, and/or combinations thereof.

The pharmaceutical composition or formulation described here may contain a cyroprotectant to stabilize a polynucleotide described herein during freezing. Exemplary cryoprotectants include, but are not limited to mannitol, sucrose, trehalose, lactose, glycerol, dextrose, and/or combinations thereof.

The pharmaceutical compositions can be administered by any suitable method, e.g., parenterally, intraventricularly, orally, topically, rectally, vaginally, nasally, buccally, or via an implanted reservoir. As used herein, the term “parenteral” includes subcutaneous, intravenous, intramuscular, intra-articular, intra-synovial, intrasternal, intrathecal, intrahepatic, intralesional and intracranial injection or infusion techniques.

Parenteral formulations can be a single bolus dose, an infusion, or a loading bolus dose followed with a maintenance dose. These compositions can be administered at specific fixed or variable intervals, e.g., twice a week or once a week. In some embodiments, the anti-CD20 antibody and/or anti-PD-1 or anti-PD-L1 antibody is administered intravenously by infusion.

In certain embodiments, the pharmaceutical compositions can be orally administered in an acceptable dosage form including, e.g., capsules, tablets, aqueous suspensions, or solutions. In certain embodiments, the pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

Dosages for the agents of the invention are described herein. Those skilled in the art will appreciate, however, that specific dosage and treatment regimen for any particular patient will depend upon a variety of factors, including the particular therapeutic agents used, the patient's age, body weight, general health, sex, and diet, and the time of administration, rate of excretion, drug combination, and the severity of the particular disease being treated. Judgment of such factors by medical caregivers is within the ordinary skill of those in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

VI. Administration of the Combination

In some embodiments, the agents (i.e., the PI3K-delta selective inhibitor, the anti-CD20 antibody, and the anti-PD-1 or anti-PD-L1 antibody, as described herein) to be used in combination in the methods described herein, are administered to a subject separately.

In some embodiments, the agents (i.e., the PI3K-delta selective inhibitor, the anti-CD20 antibody, and the anti-PD-1 or anti-PD-L1 antibody, as described herein) to be used in combination in the methods described herein, are administered to a subject sequentially, although, as noted below, the particular order of administration is not an issue.

In some embodiments, the agents are administered to a subject simultaneously or sequentially. In some embodiments, the agents are contained in the same pharmaceutical composition. In some embodiments, the agents are formulated for oral administration (e.g., TGR-1202).

In some embodiments, the combination of agents is sequentially administered in induction, treatment, and/or maintenance phases.

In some embodiments, the combination of all agents is administered simultaneously in a treatment phase. In some embodiments, there is no induction phase.

In some embodiments, two of the agents (e.g., ublituximab and TGR-1202), are administered together in order to induce a partial anti-tumor response, followed by administration of three agents (e.g., ublituximab, TGR-1202, and an anti-PD-1 or anti-PD-L1 antibody) to enhance the anti-tumor response. In some embodiments, a complete anti-tumor response (CR) is observed following administration of all agents (i.e., the PI3K-delta selective inhibitor, the anti-CD20 antibody, and the PD-1 or PD-L1 antibody, as described herein) to said subject. In some embodiments, a subject administered any of the methods described herein achieves a complete response with minimal residual disease (MRD).

In some embodiments, a subject administered any of the methods described herein achieves a partial reponse (PR) when all three agents are administered in combination. In some embodiments, a subject administered any of the methods described herein achieves a partial response (PR) or a complete response (CR) that is durable for at least two months.

In some embodiments, at least one of the agents (the PI3K-delta selective inhibitor, the anti-CD20 antibody, or the anti-PD-1 or anti-PD-L1 antibody, as described herein), is administered in a maintenance therapy in order to keep the hematological cancer from returning after successful treatment. In some embodiments, the agent is administered in maintenance therapy for an extended period of time, e.g., until unmanageable toxicity, or disease progression occurs. In some embodiments, the maintenance therapy ends when disease progression occurs.

In some embodiments, other therapeutic agents can be coformulated with and/or coadministered with the PI3K-delta selective inhibitor, the anti-CD20 antibody, and/or the anti-PD-1 or anti-PD-L1 antibody, as described herein. In some embodiments, the methods described herein further comprise administering to the subject at least one additional therapeutic agent. In some embodiments, the at least one additional therapeutic agent is selected from the group consisting of mitotic inhibitors, alkylating agents, anti-metabolites, anthracyclines, vinca alkaloids, plant alkaloids, nitrogen mustards, proteasome inhibitors, intercalating antibiotics, growth factor inhibitors, cell-cycle inhibitors, biological response modifiers, anti-hormones, angiogenesis inhibitors, anti-androgens, DNA interactive agents, purine analogues, topoisomerase I inhibitors, topoisomerase II inhibitors, tubulin interacting agents, hormonal agents, thymidilate synthase inhibitors, non-PI3K-delta tyrosine kinase inhibitors, angiogenesis inhibitors, EGF inhibitors, VEGF inhibitors, CDK inhibitors, SRC inhibitors, c-Kit inhibitors, Herl/2 inhibitors, inhibitors of myc, anti-tumor antibodies, monoclonal antibodies directed against growth factor receptors, protein kinase modulators, radioactive isotopes, immunotherapies, glucocorticoids, and combinations thereof.

In some embodiments, the at least one additional therapeutic agent is an anti-cancer agent selected from the group consisting of DNA interactive agents, such as cisplatin or doxorubicin; topoisomerase II inhibitors, such as etoposide; topoisomerase I inhibitors such as CPT-11 or topotecan; tubulin interacting agents, such as paclitaxel, docetaxel or the epothilones (for example ixabepilone), either naturally occurring or synthetic; hormonal agents, such as tamoxifen; thymidilate synthase inhibitors, such as 5-fluorouracil; and anti-metabolites, such as methotrexate; other tyrosine kinase inhibitors such as Iressa and OSI-774; angiogenesis inhibitors; EGF inhibitors; VEGF inhibitors; CDK inhibitors; SRC inhibitors; c-Kit inhibitors; Herl/2 inhibitors and monoclonal antibodies directed against growth factor receptors such as erbitux (EGF) and herceptin (Her2); and other protein kinase modulators. Other anti-cancer agents that could be used in the methods and kits of the invention will be known to those skilled in the oncology art.

In some embodiments, the at least one additional therapeutic agent is selected from the group consisting of a proteasome inhibitor, Bortezomib (Velcade®), Carfilzomib (PR-171), PR-047, disulfiram, lactacystin, PS-519, eponemycin, epoxomycin, aclacinomycin, CEP-1612, MG-132, CVT-63417, PS-341, vinyl sulfone tripeptide inhibitors, ritonavir, PI-083, (+/−)-7-methylomuralide, (−)-7-methylomuralide, lenalidomide, and combinations thereof.

In some embodiments, the at least one additional therapeutic agent is a combination of chemotherapies, known to treat hematological malignancies, such as, e.g., “CHOP” (a combination including (i) cyclophosphamide such as cytoxan, (ii) doxorubicin or other topoisomerase II inhibitors such as adriamycin, (iii) vincristine or other vincas such as oncovin; and (iv) a steroid such as hydrocortisone or prednisolone); “R-CHOP” (a combination including rituxan, cyclophosphamide, doxorubicin, vincristine, and prednisone); “ICE” (a combination including ifosfamide, carboplatin, and etoposide); “R-ICE” (a combination including rituxan, ifosfamide, carboplatin, and etoposide); “R-ACVBP” (a combination of rituximab, doxorubicin, cyclophosphamide, vindesine, bleomycin and prednisone); “DA-EPOCH-R” (a combination of dose-adjusted etoposide, doxorubicin, cyclophosphamide, vincristine, prednisone and rituximab); “R-bendamustine” (a combination of bendamustine and rituximab); “GemOx or R-GemOx” (a combination of gemcitabine and oxaliplatin, with or without rituximab); and “DHAP” (a combination including dexamethasone, cytarabine, and cisplatin).

The combination of agents comprising a PI3K-delta inhibitor, an anti-CD20 antibody, and an anti-PD-1 or anti-PD-L1 antibody (or more than one of any or all agents) can be administered in any order or at any interval as determined by those skilled in the art. For example, a PI3K-delta inhibitor of formula A, ublituximab or an anti-CD20 antibody that binds to the same epitope as ublituximab, and an anti-PD-1 or anti-PD-L1 antibody can be administered sequentially (in any order), simultaneously, or via any combination of sequential and simultaneous administrations. Any combination of a PI3K-delta inhibitor of formula A, ublituximab or an anti-CD20 antibody that binds to the same epitope as ublituximab, and an anti-PD-1 or anti-PD-L1 antibody can be administered in the same pharmaceutical compositions or in separate pharmaceutical compositions.

Administration of the combination of agents, whether simultaneous, sequential (in any order) or both, can be performed according to any number of desired intervals of minutes (e.g., 0-60 minutes), hours (e.g., 0-24 hours), days (e.g., 0-7 days), and/or weeks (e.g., 0-52 weeks), as can be determined by one of skill in the art. The dosing can also vary over time, for example, starting with a once weekly dose for a period of time (e.g., for 1, 2, 3, 4, 5, or 6 weeks) followed by dosing once every two weeks, once every three weeks, once every four weeks, once every five weeks, or once every six weeks. Dosage regimens can be adjusted to provide the optimum desired response (e.g., tumor regression or remission). Exemplary dosages and dosing intervals can also vary over time (e.g., depending upon the patient's clinical response, side effects, etc.), or during different phases of therapy (induction, treatment, or maintenance).

VII. Methods of Treating Hematological Cancer

In one aspect, the present disclosure provides a method for treating or slowing the progression of a hematological cancer, by administering to a subject in need thereof a therapeutically effective amount of: (i) at least one inhibitor of PI3K-delta, or a pharmaceutically acceptable salt, solvate or prodrug thereof; (ii) at least one anti-CD20 antibody or fragment thereof that binds to the same epitope as ublituximab; and (iii) at least one anti-PD1 or anti-PD-L1 antibody. In some embodiments, administration of at least all three agents (i.e., a PI3K-delta inhibitor, an anti-CD20 antibody, and an anti-PD1 or anti-PD-L1 antibody), occurs during a treatment phase. In some embodiments, the treatment phase is preceded by an induction phase. In some embodiments, the treatment phase is not preceded by an induction phase.

Numerous types of hematological cancers can be treated by the disclosed methods (and kits). In some embodiments, the hematological cancer is selected from the group consisting of acute lymphocytic leukemia (ALL), acute myeloid leukemia (AML), chronic lymphocytic leukemia (CLL), small lymphocytic lymphoma (SLL), multiple myeloma (MM), non-Hodgkin's lymphoma (NHL), mantle cell lymphoma (MCL), follicular lymphoma (FL), Waldenstrom's macroglobulinemia (WM), diffuse large B-cell lymphoma (DLBCL), marginal zone lymphoma (MZL), including extranodal and nodal MZL, hairy cell leukemia (HCL), Burkitt's lymphoma (BL), and Richter's transformation.

In some embodiments, the hematological cancer is selected from the group consisting of chronic lymphocytic leukemia (CLL), small lymphocytic lymphoma (SLL), non-Hodgkin's lymphoma (NHL), mantle cell lymphoma (MCL), follicular lymphoma (FL), diffuse large B-cell lymphoma (DLBCL), and marginal zone lymphoma (MZL).

In some embodiments, the cancer expresses CD20.

In some embodiments, the cancer expresses PD-1.

In some embodiments, the cancer expresses PD-L1.

In some embodiments, the cancer is refractory to chemotherapy.

In some embodiments, the cancer is refractory to non-TGR-1202 PI3K-delta inhibitors (e.g., Idelalisib or Duvelisib).

In some embodiments, the cancer is refractory to non-ublituximab anti-CD20 antibodies. In some embodiments, the cancer is refractory to rituximab.

In some embodiments, the cancer is refractory to any agent described herein, i.e., an anti-CD20 antibody, a PI3K delta selective inhibitor, or anti-PD-1 or anti-PD-L1 antibody, when said agent was previously administered individually to a subject (i.e., the agent was used as a monotherapy).

In some embodiments, the cancer has relapsed.

In some embodiments, the human subject has one or more genetic mutations selected from the group consisting of 17p del, 11q del, p53, unmutated IgVH together with ZAP-70+ and/or CD38+, and trisomy 12.

The PI3K-delta inhibitor, the anti-CD20 antibody, and the anti-PD-1 or anti-PD-L1 antibody used in the methods (and kits) described herein can be administered in any order or at any interval as determined by one of skill in the art. For example, the PI3K-delta inhibitor, the anti-CD20 antibody, and the anti-PD-1 or anti-PD-L1 antibody can be administered sequentially (in any order), simultaneously, or via any combination of sequential and simultaneous administrations. The PI3K-delta inhibitor, the anti-CD20 antibody, and the anti-PD-1 or anti-PD-L1 antibody can be administered in the same pharmaceutical composition or in separate pharmaceutical compositions.

Administration of a PI3K-delta inhibitor, an anti-CD20 antibody, and an anti-PD-1 or anti-PD-L1 antibody, whether simultaneous, sequential (in any order) or both, can be performed according to any number of desired intervals of minutes (e.g., 0-60 minutes), hours (e.g., 0-24 hours), days (e.g., 0-7 days), and/or weeks (e.g., 0-52 weeks) as can be decided and determined by one of skill in the art. Exemplary dosages and dosing intervals can also vary over time (e.g., depending upon the patient's clinical response, side effects, etc.), or during different phases of therapy (induction, treatment, or maintenance).

In some embodiments, the treatment phase in the methods described herein lasts up to about 18 weeks, up to about 17 weeks, up to about 16 weeks, up to about 15 weeks, up to about 14 weeks, up to about 13 weeks, or up to about 12 weeks. In some embodiments, the treatment phase lasts about 12 weeks.

In some embodiments, in the treatment phase, the PI3K-delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate, or prodrug thereof, which is administered daily at a dose ranging from about 200 to about 1200 mg, about 400 to about 1200 mg, about 400 to about 1000 mg, about 400 to about 800 mg, about 500 to about 1200 mg, about 500 to about 1000 mg, about 500 to about 800 mg, about 400 mg, about 500 mg, about 600 mg, about 700 mg, about 800 mg, about 900 mg, about 1000 mg, or about 1200 mg. In some embodiments, the PI3K-delta inhibitor, or a pharmaceutically acceptable salt, solvate or prodrug thereof, is micronized and/or formulated for oral administration. In some embodiments, the PI3K-delta inhibitor, or a pharmaceutically acceptable salt, solvate or prodrug thereof, is administered under a fed condition. In some embodiments, the PI3K-delta inhibitor, or a pharmaceutically acceptable salt, solvate or prodrug thereof, is administered daily at about 800 mg during the treatment phase. In a preferred embodiment, the PI3K-delta inhibitor is the PTSA salt of (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, which is also called TGR-1202 or umbralisib tosylate.

In some embodiments, in the treatment phase, the anti-CD20 antibody is ublituximab, which is administered at a dose from: about 450 to about 1200 mg, about 450 to about 1000 mg, about 500 to about 1200 mg, about 500 to about 1000 mg, about 500 to about 900 mg, about 600 to about 1200 mg, about 600 to about 1000 mg, about 600 to about 900 mg, about 500 mg, about 600 mg, about 700 mg, about 750 mg, about 800 mg, about 900 mg, about 1000 mg, about 1100 mg, about 1200 mg, about once every 4 to 7 weeks, about once every 5 to 7 weeks, once every 5 to 6 weeks, about once a week, about once every 2 weeks, about once every 3 weeks, about once every 4 weeks, about once every 5 weeks, about once every 6 weeks, or about once every 7 weeks.

In some embodiments, ublituximab is administered at a dose of about 900 mg about once every 6 weeks. In some embodiments, the first dose of ublituximab is administered on day 1 of the sixth week after the treatment phase is initiated. In some embodiments, ublituximab is formulated for intravenous infusion.

In some embodiments, in the treatment phase, the anti-PD-1 antibody is pembrolizumab, which is administered at about 100 to about 300 mg, about 100 to about 200 mg, about 100 mg, about 150 mg, about 200 mg, about 250 mg or about 300 mg about once every 2 to 4 weeks, or about once every 3 to 4 weeks, or about once every 2 weeks, about once every 3 weeks, or about once every 4 weeks. In some embodiments, pembrolizumab is administered at a dose of about 100 mg or 200 mg about once every 2, 3, or 4 weeks. In some embodiments, the first dose of pembrolizumab is administered on day 1 when the treatment phase is initiated. In some embodiments, pembrolizumab is formulated for intravenous infusion.

In some embodiments, in the treatment phase, the anti-PD-L1 antibody is atezolizumab, which is administered at a dose of about 500 mg to about 1500 mg, every 2 to 5 weeks. In some embodiments, the atezolizumab is administered at a dose of about 1200 mg every 3 weeks. In some embodiments, the first dose of atezolizumab is administered on day 1 when the treatment phase is initiated. In some embodiments, atezolizumab is formulated for intravenous infusion.

In some embodiments, the methods described herein further comprise an induction phase, prior to the treatment phase. The induction phase lasts up to about 12 weeks, up to about 11 weeks, up to about 10 weeks, up to about 9 weeks, or up to about 8 weeks. In some embodiments, the induction phase lasts about 8 weeks.

In some embodiments, in the induction phase, the PI3K-delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate or prodrug thereof, which is administered at a daily dose range of about 200 to about 1200 mg, about 400 to about 1200 mg, about 400 to about 1000 mg, about 400 to about 800 mg, about 500 to about 1200 mg, about 500 to about 1000 mg, about 500 to about 800 mg, about 400 mg, about 500 mg, about 600 mg, about 700 mg, about 800 mg, about 900 mg, about 1000 mg, or about 1200 mg. In some embodiments, the P13K-delta inhibitor, or a pharmaceutically acceptable salt, solvate or prodrug thereof, is micronized and/or formulated for oral administration.

In some embodiments, the PI3K-delta inhibitor, (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate or prodrug thereof, is administered under a fed condition. In a preferred embodiment, the PI3K-delta inhibitor, or a pharmaceutically acceptable salt, solvate or prodrug thereof, is administered daily at about 800 mg daily during the induction phase.

In some embodiments, in the induction phase, the anti-CD20 antibody is ublituximab, which is administered at about 450 to about 1200 mg, about 450 to about 1000 mg, about 500 to about 1200 mg, about 500 to about 1000 mg, about 500 to about 900 mg, about 600 to about 1200 mg, about 600 to about 1000 mg, about 600 to about 900 mg, about 500 mg, about 600 mg, about 700 mg, about 800 mg, about 900 mg, or about 1000 mg about once every 1 to 3 weeks, about once every 2 to 3 weeks, about once every 1 to 2 weeks, about once every 1 week, about once every 2 weeks, or about once every 3 weeks. In some embodiments, ublituximab is administered at a dose of about 900 mg about once every 1 or 2 weeks during the induction phase. In some embodiments, the first dose of ublituximab is administered on day 1 of the induction phase. In some embodiments, the first dose of ublituximab is divided into 2 or 3 sub-doses to be administered in 2 or 3 consecutive days during the induction phase, or is divided into 2 sub-doses to be administered in 2 consecutive days during the induction phase. In some embodiments, the first sub-dose of ublituximab comprises up to 150 mg of ublituximab. In some embodiments, the second sub-dose of ublituximab comprises up to 750 mg of ublituximab. In some embodiments, ublituximab is formulated for intravenous infusion.

In some embodiments, the methods described herein further comprise a maintenance phase, following the treatment phase. The maintenance phase may last as long as a clinical benefit is observed, or until unmanageable toxicity or disease progression occurs. In some embodiments, the maintenance phase ends when disease progression occurs. In some embodiments, the maintenance phase lasts at least 3 weeks, at least 6 weeks, at least 9 weeks, at least 12 weeks, or at least 15 weeks.

In some embodiments, in the maintenance phase, the PI3K-delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate or prodrug thereof, which is administered at a daily dose range of about 200 to about 1200 mg, about 400 to about 1200 mg, about 400 to about 1000 mg, about 400 to about 800 mg, about 500 to about 1200 mg, about 500 to about 1000 mg, about 500 to about 800 mg, about 400 mg, about 500 mg, about 600 mg, about 700 mg, about 800 mg, about 900 mg, about 1000 mg, or about 1200 mg. In some embodiments, the PI3K-delta inhibitor, or a pharmaceutically acceptable salt, solvate or prodrug thereof, is micronized and/or formulated for oral administration. In some embodiments, the PI3K-delta inhibitor, or a pharmaceutically acceptable salt, solvate or prodrug thereof, is administered under a fed condition. In a preferred embodiment, the PI3K-delta inhibitor, or a pharmaceutically acceptable salt, solvate or prodrug thereof, is administered daily at about 800 mg during the maintenance phase.

While not wishing to be bound by theories, it is believed that the addition of an anti-PD-1 antibody (e.g., pembrolizumab) or an anti-PD-L1 antibody (e.g., atezolizumab or CK-301) in the treatment phase can enhance the efficacy of host T cells to induce apoptosis in hematological cancer patients following an induction phase in which a combination of an anti-CD20 antibody (e.g., ublituximab) and a PI3K-delta inhibitor (e.g., TGR-1202) is administered.

VIII. Kits

In one aspect, the present disclosure also provides a kit for treating a subject afflicted with a hematological cancer, the kit comprising: (i) a single dose or multiple doses of an anti-CD20 antibody or fragment thereof that binds to the same epitope as ublituximab; (ii) a single dose or multiple doses of a PI3K-delta inhibitor, wherein the PI3-delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate or prodrug thereof; (iii) a single dose or multiple doses of an anti-PD1 or anti-PD-L1 antibody; and (iv) instructions for using the agents (i)-(iii) according to the methods described herein.

In some embodiments, the anti-CD20 antibody in the kit is ublituximab or an anti-CD20 antibody (or fragment thereof) that binds to the same epitope as ublituximab. In some embodiments, the anti-CD20 antibody in the kit is ublituximab. In some embodiments, the single dose of ublituximab contains from about 50 to about 1200 mg, about 100 to about 1000 mg, about 150 to about 900 mg, about 250 to about 1200 mg, about 250 to about 900 mg, about 350 to about 1200 mg, about 350 to about 900 mg, about 450 to about 1200 mg, about 450 to about 900 mg, about 550 to about 1200 mg, about 550 to about 900 mg, about 650 to about 1200 mg, about 650 to about 900 mg, about 750 to about 1200 mg, about 750 to about 900 mg, or about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg of ublituximab. In some embodiments, the ublituximab dose is formulated for intravenous infusion.

In some embodiments, the PI3K-delta inhibitor in the kit is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate or prodrug thereof. In some embodiments, the PI3K-delta inhibitor in the kit is micronized. In some embodiments, the PI3K-delta inhibitor is formulated for oral administration. In some embodiments, the single dose P13K-delta inhibitor contains from about 100 to about 1200 mg, about 200 to about 1000 mg, about 300 to about 1000 mg, about 400 to about 800 mg, about 100 mg, 200 mg, about 300 mg, about 400 mg, about 500 mg, about 600 mg, about 700 mg, about 800 mg, about 900 mg, about 1000 mg, or about 1200 mg of (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate or prodrug thereof. In some embodiments, the P13K-delta inhibitor in the kit is the PTSA salt of (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, which is also known as TGR-1202 (umbralisib tosylate). In some embodiments, the single dose TGR-1202 is a tablet or a capsule for oral administration.

In some embodiments, the anti-PD-1 antibody in the kit is, e.g., nivolumab, pembrolizumab, or pidilizumab, or any other anti-PD-1 antibody described herein or known to those skilled in the art. In some embodiments, the anti-PD-1 antibody is pembrolizumab. In some embodiments, the single dose contains about 25 mg to about 300 mg, about 50 mg to 300 mg, about 100 to about 300 mg, about 150 to 300 mg, about 200 to 300 mg, or about 25 mg, about 50 mg, about 75 mg, about 100 mg, about 125 mg, about 150 mg, about 173 mg, about 200 mg, or about 250 mg, or about 300 mg of pembrolizumab. In some embodiments, the single dose pembrolizumab is formulated for intravenous infusion.

In some embodiments, the anti-PD-L1 antibody in the kit is, e.g., durvalumab, BMS-936559, atezolizumab, avelumab, or any other anti-PD-L1 antibody described herein (i.e., CTI-07, CTI-09, CTI-48, CTI-49, CTI-50, CTI-76, CTI-77, CTI-78, CTI-57, or CTI-58), or known to those skilled in the art. In some embodiments, the anti-PD-L1 antibody is atezolizumab. In some embodiments, the single dose contains about 1200 mg of atezolizumab. In some embodiments, the single dose atezolizumab is formulated for intravenous infusion.

In some embodiments, the kit further comprises an additional anti-cancer agent. In some embodiments, the additional anti-cancer agent is a chemotherapeutic agent selected from the group consisting of DNA interactive agents, such as cisplatin or doxorubicin; topoisomerase II inhibitors, such as etoposide; topoisomerase I inhibitors such as CPT-11 or topotecan; tubulin interacting agents, such as paclitaxel, docetaxel or the epothilones (for example ixabepilone), either naturally occurring or synthetic; hormonal agents, such as tamoxifen; thymidilate synthase inhibitors, such as 5-fluorouracil; and anti-metabolites, such as methotrexate; other tyrosine kinase inhibitors such as Iressa and OSI-774; angiogenesis inhibitors; EGF inhibitors; VEGF inhibitors; CDK inhibitors; SRC inhibitors; c-Kit inhibitors; Her1/2 inhibitors and monoclonal antibodies directed against growth factor receptors such as erbitux (EGF) and herceptin (Her2); and other protein kinase modulators.

One skilled in the art will readily recognize that the disclosed combination of agents (e.g., anti-CD20 antibody, PI3K-delta inhibitor, and anti-PD-1 or anti-PD-L1 antibody) described herein for use in the methods described herein can be readily incorporated into one of the established kit formats that are well-known in the art.

The present invention is further illustrated by the following examples which should not be construed as further limiting. The contents of all patent and non-patent references cited throughout this application are expressly incorporated herein by reference in their entireties.

### EXAMPLES

### Example 1

### Phase I/II Study of Pembrolizumab in Combination with Ublituximab (TG-1101) and Umbralisib Tosylate (TGR-1202) in Patients with Relapsed/Refractory (r/r) Chronic Lymphocytic Leukemia (CLL)

Background

In the U.S., an estimated 20,110 new cases of CLL will be reported for 2017 with deaths totaling 4,660 due to the disease according to the American Cancer Society. CLL affects mainly older adults, who account for one third of all diagnosed cases of leukemia, and is characterized by the accumulation of clonal mature B lymphocytes in the blood, bone marrow, and secondary lymphoid tissues. CLL is a heterogeneous disease, with several higher risk cytogenetic abnormalities that are generally more difficult to treat, including 17p deletion, P53 gene mutation, and 11q deletion. See Dohner, H. et al., N Eng J Med 343:1910-1916(2000).

CLL is a disorder that utilizes immune dysregulation to evade cell death and promote tumor survival. Chemotherapy regimens in combination with monoclonal antibody therapy comprise the current standard of care for patients with CLL. Frontline therapy for patients with CLL who are able to tolerate chemotherapy generally includes anti-CD20 monoclonal antibody rituximab, in combination with either fludarabine and cyclophosphamide, or bendamustine. Chlorambucil in combination with an anti-CD20 monoclonal antibody is also used. See Fischer et al., ASH Annual Meeting, Abstract No. 435 (2012); Eichhorst, B. et al., Blood 122:526-526 (2013). In addition to rituximab, other anti-CD20 antibodies including ofatumumab and obinutuzumab have also been considered for the treatment of CLL (see e.g., Goede, V. et al., N Eng JMed 370:1101-1110 (2014)).

Treatment using ibrutinib (IMBRUVICA®) has demonstrated clinical efficacy in patients with CLL. See Byrd, J. C. et al., N Engl JMed 371:213-223 (2014). A combination treatment using idelalisib (ZYDELIG®) and rituximab for CLL patients with relapsed disease has also been reported. See Furman, R. R. et al., N Eng JMed 370:997-1007 (2014). Although idealisib-rituximab combination treatment and ibrutinib demonstrated clinical activity in r/r CLL patients, neither treatment regimen was curative and both rarely resulted in patients obtaining a complete response or minimal residual negative disease in the peripheral blood or bone marrow.

Recent data suggests that PD-1 and its ligands PD-L1/PD-L2 mediate immune evasion in CLL. However, recent studies by Ding, W. et al., “Pembrolizumab in patients with CLL and Richter transformation or with relapsed CLL,” Blood 129: 3419-3427 (2017), demonstrate that pembrolizumab (“pembro”) alone is ineffective in patients with CLL (ORR 0%, median PFS 2.4 months). In 5 patients with r/r CLL, 3 responded to the combination of ibrutinib/nivolumab. Jain, N. et al., “Nivolumab Combined with Ibrutinib for CLL and Richter Transformation: A Phase II Trial,” 58^{th} ASH Annual Meeting; San Diego, Calif.; Dec. 2-6, 2016. Abstract No. 59. A key interaction may exist between PI3K signaling and immune checkpoint surveillance by which inhibition of PI3K decreases PD-L1 tumor expression. In this Example, the safety and activity of umbralisib tosylate, a next generation, highly-specific PI3K-δ inhibitor, was tested in combination with pembro and the glycoengineered anti-CD20 monoclonal antibody ublituximab in r/r CLL. It is believed that this is the first report on a combination of a PD-1 inhibitor with a PI3K-δ inhibitor, and an assessment of possible synergistic activity.

Study Design

In this Phase I/II multi-center clinical study, pembrolizumab, TGR-1202 (umbralisib tosylate), and ublituximab, were administered to 10 adult patients with CLL (1 with Richter's transformation (RT)) with relapsed or refractory disease requiring therapy.

The study was conducted to assess the safety of pembrolizumab+TGR-1202+ ublituximab following the combination induction treatment of ublituximab+TGR-1202. Thus, safety of the triplet combination was a primary endpoint.

The study also evaluated the clinical efficacy of pembrolizumab+TGR-1202+ ublituximab following the combination induction treatment of ublituximab+TGR-1202 in patients with relapsed-refractory CLL. Thus, efficacy of the triplet combination was a secondary endpoint. Efficacy was measured as overall response rate (ORR), complete response rate (CRR), and progression free survival (PFS) for this cohort.

Response and efficacy endpoints were defined per the 2008 International Workshop on Chronic Lymphocytic Leukemia (IWCLL) guidelines. See Hallek, M. et al., Blood 111:5446-5456 (2008).

Patients must have met all of the following inclusion criteria to be eligible for participation in this study:

1. CLL Cohorts—Diagnosis of B-cell CLL, with diagnosis established according to IWCLL criteria documented within the medical records. Patients must have received at least 2 cycles of one prior standard treatment regimen (prior anti-CD20 antibody or cytotoxic drugs, including investigational or commercially available therapies, may have been administered as single agents or as components of combination therapies).

2. RT Cohorts—Histologically confirmed diagnosis of Richter's transformation of CLL. Patients must have received at least one prior line of therapy for either CLL or RT.

3. CLL Cohorts—CLL that warrants treatment consistent with accepted IWCLL criteria for initiation of therapy. Any of the following conditions constitute CLL that warrants treatment:
a. Evidence of progressive marrow failure as manifested by the onset or worsening of anemia and/or thrombocytopenia, or
b. Massive (i.e., lower edge of spleen≥6 cm below the left costal margin), progressive, or symptomatic splenomegaly, or
c. Massive (i.e., ≥10 cm in the longest diameter), progressive, or symptomatic lymphadenopathy, or
d. Progressive lymphocytosis in the absence of infection, with an increase in blood absolute lymphocyte count (ALC) ≥50% over a 2-month period or lymphocyte doubling time of <6 months (as long as initial ALC was ≥30,000/0, or
e. Autoimmune anemia and/or thrombocytopenia that is poorly responsive to corticosteroids or other standard therapy, or
f. Constitutional symptoms, defined as any one or more of the following disease-related symptoms or signs occurring in the absence of evidence of infection:
   i. Unintentional weight loss of ≥10% within the previous 6 months, or
   ii. Significant fatigue (≥Grade 2), or
   iii. Fevers >100.5° F. or 38.0° C. for ≥2 weeks, or
   iv. Night sweats for >1 month.
4. Adequate organ system function, defined as follows:
   a. Absolute neutrophil count (ANC) >750/platelet count >40,000,
   b. Total bilirubin ≤1.5 times the upper limit of normal (ULN),
   c. Alanine aminotransferase (ALT) and aspartate aminotransferase (AST) ≤2.5× ULN if no liver involvement or ≤5× the ULN if known liver involvement,
   d. Calculated creatinine clearance >30 mL/min (as calculated by the Cockcroft-Gault formula).

5. ECOG performance status ≤2.

6. Male or female ≥18 years of age.

7. Ability to swallow and retain oral medication.

8. Female subjects of child bearing potential must be surgically sterile, be post-menopausal (per institutional guidelines), or must agree to use medically acceptable contraception for two weeks before beginning study drug, during the period of therapy and for 30 days following the last dose of study drug. All female subjects with reproductive potential must have a negative pregnancy test within 3 days prior to Cycle 1, Day 1. Women or men of reproductive potential may not participate unless they agree to use medically acceptable contraception. Subjects of child bearing potential must use two forms of medically acceptable contraception, to include: condoms, diaphragms, cervical cap, an intra-uterine device (IUD), surgical sterility (tubal ligation or a partner that has undergone a vasectomy), or oral contraceptives, OR must agree to completely abstain from heterosexual intercourse for two weeks before beginning study drug, during participation in this study, and for 30 days after the final dose of study drug. Abstinence at certain times of the cycle only, such as during the days of ovulation, after ovulation and withdrawal are not acceptable methods of birth control. The study doctor must approve the form of birth control. Female subjects should not become pregnant while participating in this research study or for 30 days following the last dose of study drug. Male subjects should not father a child or donate sperm while in this research study or for 30 days following the last dose of study drug.

9. Willingness and ability to comply with study and follow-up procedures, and give written informed consent.

Patients who met any of the following exclusion criteria were not enrolled in this study:

1. Patients receiving cancer therapy (i.e., chemotherapy, radiation therapy, immunotherapy, biologic therapy, hormonal therapy, surgery and/or tumor embolization) or any investigational drug within 14 days of enrollment.

2. Evidence of chronic active Hepatitis B (HBV not including patients with prior hepatitis B vaccination; or positive serum Hepatitis B antibody), or chronic active Hepatitis C infection (not including negative Hepatitis C confirmed by PCR), cytomegalovirus (CMV), or known history of HIV.

3. Evidence of ongoing systemic bacterial, fungal or viral infection, except localized fungal infections of skin or nails (patients may be receiving prophylactic antiviral or antibacterial therapies at investigator discretion).

4. Any severe and/or uncontrolled medical conditions or other conditions that could affect their participation in the study such as:
a. Symptomatic, or history of documented congestive heart failure (NY Heart Association functional classification III-IV),
b. Myocardial infarction within 3 months of randomization,
c. QTcF >470 msec,
d. Angina not well-controlled by medication,
e. Poorly controlled or clinically significant atherosclerotic vascular disease including cerebrovascular accident (CVA), transient ischemic attack (TIA), angioplasty, cardiac/vascular stenting within 3 months of randomization.

5. Malignancy within 2 years of study enrollment except for adequately treated basal, squamous cell carcinoma or non-melanomatous skin cancer, carcinoma in situ of the cervix, superficial bladder cancer not treated with intravesical chemotherapy or BCG within 6 months, localized prostate cancer and PSA <1.0 mg/dL on 2 consecutive measurements at least 3 months apart with the most recent one being within 4 weeks of study entry.

6. Patients with an active autoimmune disorder (with the exception of autoimmune hemolytic anemia or idiopathic thrombocytopenic purpura (ITP)).

Dosing Schedules

Ten (10) subjects were treated based on the following 3-phase dosing schedule.
Induction Phase (Cycles 1 and 2, with each cycle=28 days)

Eligible patients received TGR-1202 (umbralisib tosylate), 800mg orally daily.

Eligible patients also received ublituximab, 900 mg by intravenous (IV) infusion, on days 1, 8, and 15 of cycles 1 and 2. For cycle 1, ublituximab dosing was split over two days to minimize the risk of tumor lysis syndrome and infusion related reactions (up to 150 mg on day 1 and 750 mg on day 2).
Treatment or Consolidation Phase (Cycles 3-6, with each cycle=21 days)

In the treatment phase, pembrolizumab was initiated every 3 weeks in combination with ublituximab and daily TGR-1202, according to the following dosing schedule:

Ublituximab: IV infusion 900 mg on day 15 of cycles 4 and 6.

TGR-1202: 800 mg oral daily dose.

Pembrolizumab: Dose level 1: 100 mg IV infusion of pembrolizumab was administered every three weeks for four cycles of therapy. Pembrolizumab was administered on day 1 of each 21 day cycle. Dose level 2: 200 mg IV infusion pembrolizumab was administered every three weeks for four cycles of therapy. Pembrolizumab was administered on day 1 of each 21 day cycle.
Maintenance Phase (Cycles 7+, with each cycle=28 days)

Upon completion of cycle 6, patients continued TGR-1202 (umbralisib tosylate), 800 mg daily, as a maintenance therapy until progressive disease (PD) or unacceptable toxicity. Response assessments were based on the IWCLL 2008 criteria and performed after umbralisib tosylate+ublituximab (2 months), umbralisib tosylate, ublituximab, pembro (6 months) and during maintenance with umbralisib tosylate at month 12. Peripheral blood and/or bone marrow biopsy were obtained for correlative analyses at screening, month 2, and month 6. For correlative analysis, mononuclear cells were enriched and cryopreserved. Cryopreserved cells were subjected to multicolor immunophenotyping to analyze: B cells (CD5, CD38, CD3, HLA-DR, CD19, PDL2, CD27, PDL1 and viability) and T/NK cells (CD8, CD56, CCCR7, CD3, CD4, IgG4, CD19, TIM-3, CD25, PD1 and viability).

Following completion of the induction phase, a response assessment and correlative sampling was performed and then patients received pembrolizumab in combination with TGR-1202 and ublituximab. This unique design of two separates phases (ublituximab′TGR-1202 followed by pembrolizumab+TGR-1202+ ublituximab) was designed to study for the first time in CLL in vivo the importance and activity of PD-1 signaling blockade in CLL, and at the same time assess for added toxicity of pembrolizumab to the combination of ublituximab and TGR-1202.

The study used a traditional 3+3 phase I study design for dose escalation in two cohorts of 3-6 patients per dose level. If none of the first three subjects at the first pembrolizumab dose level (100 mg) experienced dose limiting toxicity (DLT), then dose escalation proceeded to the next level (200 mg). A minimum of three subjects were evaluated at the first dose level if no dose limiting toxicity was observed. No DLTs were reported among the 3 subjects at the 100 mg pembrolizumab dose level. One DLT of elevated ALT/AST among the first 3 enrolled subjects was reported at the 200 mg pembrolizumab dose level, necessitating enrollment of an additional 3 subjects at this dose level. No other DLTs were reported.

Patients have and will continue treatment until the occurrence of definitive disease progression, unacceptable toxicity, or withdrawal from the study for other reasons. Patients who discontinue study treatment for reasons other than progression will continue to be followed for progression and/or survival for a one year period of time from the time of enrollment. Table 6 lists all of the required assessments performed at each study visit.

TABLE 6
Study Assessments and Treatment Schedule for CLL Patients

| Cycles 1, 2, | | | | | | | | | | | | |
|---|---|---|---|---|---|---|---|---|---|---|---|---|
| and 7+ = 28 | | | | | | | | | | | | |
| days | Screening | | | | | | | | Cycles | Cycles | | |
| Cycles ≥ 3-6 = | D −28 | Cycle 1¹ | | | | Cycle 2² | | | 3, 4, 5, 6² | 4 & 6² | Maintenance³ | End of |
| 21 days | to D0 | D1 | D2 | D8 | D15 | D1 | D8 | D15 | D1 | D15 | D1 | Treatment⁴ |
| Informed | X | | | | | | | | | | | |
| consent | | | | | | | | | | | | |
| Medical | X | | | | | | | | | | | |
| history | | | | | | | | | | | | |
| ECOG | X | X | | | | X | | | X | | X | |
| Performance | | | | | | | | | | | (q3 mo)⁷ | |
| Status | | | | | | | | | | | | |
| Physical | X | X | | | | X | | | X | | X | |
| Examination | | | | | | | | | | | (q3 mo)⁷ | |
| Vital signs | X | X | X | X | X | X | X | X | X | X | X | |
| | | | | | | | | | | | (q3 mo)⁷ | |
| Hematology | X | X | X | X | X | X | X | X | X | X | X | |
| | | | | | | | | | | | (q3 mo)⁷ | |
| Chemistry, | X | X | | | | X | | | X | X | X | |
| LDH, Uric acid | | | | | | | | | | | (q3 mo)⁷ | |
| EKG | X | | | | | | | | | | | |
| Ublituximab | | X* | X* | X | X | X | X | X | | X | | |
| 900 mg | | | | | | | | | | | | |
| Hep B/C | X | | | | | | | | | | | |
| serology, quant | | | | | | | | | | | | |
| Igs, Coombs | | | | | | | | | | | | |
| test | | | | | | | | | | | | |
| CT imaging | X⁵ | | | | | | | | X⁶ | | X⁶ | |
| with IV | | | | | | | | | | | | |
| contrast (neck, | | | | | | | | | | | | |
| chest, | | | | | | | | | | | | |
| abdomen, | | | | | | | | | | | | |
| pelvis) | | | | | | | | | | | | |
| Serum | X⁸ | | | | | | | X | X⁶ | | | |
| Pregnancy Test | | | | | | | | | | | | |
| Study labs | X⁹ | | | | | | | X⁹ | | X⁹ | | X |
| (PB/BM) | (PB/BM) | | | | | | | (PB) | | (PB/ | | (PB/BM) |
| | | | | | | | | | | BM) | | |
| TGR-1202 | | Once Daily (until disease progression) | | | | | | | | | | |
| (800 mg) | | | | | | | | | | | | |
| Pembrolizumab | | | | | | | | | X | | | |
| Response | | | | | | | | | X⁶ | | X⁶ | |
| Assessment | | | | | | | | | | | | |
| Adverse Event | | X | X | X | X | X | X | X | X | | X | X |
| Assessment | | | | | | | | | | | (q3 mo)⁷ | |
| Conmed | | X | X | X | X | X | X | X | X | | X | |
| Assessment | | | | | | | | | | | (q3 mo)⁷ | |

*150 mg on Day 1 infusion and 750 mg on Day 2 infusion
¹Treatment Administration +/−1 day window. Physical Exam, Vital Signs, ECOG PS, Hematology and Serum Chem visit days have −1 day window.
²Treatment Administration +/−3 day window. Physical Exam, Vital Signs, ECOG PS, Hematology and Serum Chem visit days have a −3 day window for Cycles 2 through 6.
³Treatment Administration and labs or other assessments +/−7 day window.
⁴If clinically significant adverse event or abnormal result is observed that is not resolved by the end-of treatment visit, continue to monitor and record up through 30 days after study drug discontinuation.
⁵Baseline CT scan within 30 days prior to Cycle 1/Day 1.
⁶CT Scan/Response Assessment +/−7 day window. CT Scan/Response assessment week 8 (end of cycle 2), week 24 (end of cycle 6), after 12 months (week 52) on study and then at investigator discretion after 12 months, per standard of care.
⁷Visits at least every 3 months or as per investigator discretion.
⁸Serum pregnancy test at least 72 hours prior to Cycle 1/Day 1. If patient has experienced menopause or no longer has uterus/ovaries, this can be omitted.
⁹Peripheral Blood and Bone Marrow sample performed within 30 days prior to cycle 1. Peripheral Blood sample only within 14 days prior to completion of cycle 2. This can be collected on Cycle 3 day 1 pre-pembrolizumab. Peripheral Blood and Bone Marrow sample within 7 days following completion of Cycle 6 only. Bone Marrow sample will be used also for efficacy evaluation as needed.

Method Assessment

In addition to clinical examination, imaging-based evaluation was used in this study on all patients enrolled. CT scan was the preferred method for radiographic tumor assessment, however, MRI scanning may be used in patients for whom this may be a preferred alternative. If MRI is performed, a non-contrast CT of the chest should be performed. Contrast-enhanced scanning is preferred, but iodine-containing or gadolinium contrast material may be omitted in patients for whom use of a contrast agent would be medically contraindicated. Chest x-ray, ultrasound, endoscopy, laparoscopy, PET, radionuclide scans, or tumor markers will not be considered for response assessment.

For radiographic evaluations, the same method of assessment and the same technique (e.g., scan type, scanner, patient position, dose of contrast, injection/scan interval) was used to characterize each identified and reported lesion at baseline and during study treatment and follow-up. However, if a patient was imaged without contrast at baseline, subsequent assessments were performed with contrast, unless the patient could not tolerate the contrast.

Target Lesions

At baseline, up to 6 lymph nodes were selected as target lesions that would be used to quantitate disease status during study treatment. Ideally, the target lesions were located in disparate regions of the body. Only peripheral nodes need be selected as target lesions. However, it is optimal if mediastinal and retroperitoneal areas of disease are assessed whenever these sites are involved.

Target lesions were measured and recorded at baseline and as per the study assessment schedule. The cross-sectional dimensions (the largest cross-sectional diameter, i.e., the LD×LPD) were recorded (in cm) for each target lesion. The product of the perpendicular diameters (PPD) (in cm²) for each target lesion and the sum of the products (SPD) (in cm²) for all target lesions were calculated and recorded. The baseline SPD were used as references by which objective tumor response was characterized during treatment. The nadir LD of individual lesions and the nadir SPD will be used as references by which CLL progression will be characterized. All LD and LPD diameters were reported in centimeters and all PPDs and SPDs were reported in centimeters squared.

A nodal mass was may be selected as a nodal target lesion if it is both abnormal and measurable at baseline. A lymph node lesion was considered abnormal if it had a single diameter that is >1.5 cm and is considered measurable if it had 2 perpendicular diameters that can be accurately measured in cross section with the LD being ≥1.0 cm and the LPD also being ≥1.0 cm.

At follow-up time points, the LDs for individual lesions and the SPD of all nodal target lesions were considered. Because nodal target lesions that have one or both diameters >0 cm and <1.0 cm cannot be reliably measured, a default value of 1.0 cm was assigned for each diameter that meets these criteria and the resulting PPD was used in SPD calculations. Based on this convention, a CR may be achieved even if an SPD value is >0 cm² (i.e., if all lymph nodes measure <1.0 cm²).

A new node that measures >1.5 cm in the LD and >1.0 cm in the LPD was considered progressive disease (PD).

In cases where a large lymph node mass split into multiple components, all subcomponents regardless of size were used in calculating the SPD. Progression of the lesion was based on the SPD of sub-components. Lesion sub-components will have the true PPDs calculated. Similarly, lesion sub-components that are visible but neither abnormal nor measurable will have the default PPD of 1.0 cm² (1.0 cm×1.0 cm) used in calculating the SPD.

If lesions merged, a boundary between the lesions was established so the LD of each individual lesion can continue to be measured. If the lesions have merged in a way that they can no longer be separated by this boundary, the newly merged lesion was measured bi-dimensionally.

Spleen and Liver

Both the spleen and liver were assessed by CT/MRI scan and by physical examination at baseline and as per the study assessment schedule. The baseline and nadir values for the longest vertical dimension (LVD) of each organ were used as reference to further characterize the objective tumor response of the measurable dimensions of the CLL during treatment. All spleen and liver LVD measurements were recorded in centimeters.

By imaging, the spleen was considered enlarged if it was >12 cm in LVD, with the LVD being obtained by multiplying the number of sections on which the spleen is visualized by the thickness of the sections (e.g., if the spleen is seen in 14 contiguous cross-sectional images with 0.5-cm thickness, the LVD is recorded as 7 cm).

For patients with splenomegaly at baseline or at the splenic LVD nadir, respective response and progression evaluations of the spleen considered only changes relative to the enlargement of the spleen at baseline or nadir, not changes relative to the total splenic LVD.

A 50% decrease (minimum 2 cm decrease) from baseline in the enlargement of the spleen in its LVD or decrease to ≤12 cm by imaging was required for declaration of a splenomegaly response. Conversely, an increase in splenic enlargement by ≥50% from nadir (minimum increase of 2 cm) was required for declaration of splenic progression. By imaging, the liver was considered enlarged if it was >18 cm in LVD.

A 50% decrease (minimum 2 cm decrease) from baseline in the enlargement of the liver in its LVD or decrease to ≤18 cm was required for declaration of a hepatomegaly response. Conversely, an increase in liver enlargement by ≥50% from nadir (minimum increase of 2 cm) was required for declaration of hepatic progression.

Non-Target Lesions

Any other measurable and abnormal nodal lesions not selected for quantitation as target lesions were considered non-target lesions. In addition, non-measurable evidence of CLL such as nodal lesions with both diameters <1.0 cm, extra-nodal lesions, bone lesions, leptomeningeal disease, ascites, pleural or pericardial effusions, lymphangitis of the skin or lung, abdominal masses that are not confirmed and followed by imaging techniques, cystic lesions, previously irradiated lesions, and lesions with artifacts were considered as non-target disease.

The presence or absence of non-target disease was recorded at baseline and at the stipulated intervals during treatment. If present at baseline, up to 6 non-target lesions should be recorded. The non-target disease at baseline was used as a general reference to further characterize regression or progression of CLL during assessments of the objective tumor response during treatment. Measurements were not required, and these lesions were followed as “present” or “absent.”

Definitions of Tumor Response and Progression

To satisfy criteria for a complete response (CR), all of the following criteria must have been met:

(1) No evidence of new disease.

(2) ALC in peripheral blood of <4×10⁹/L.

(3) Regression of all target nodal masses to normal size ≤1.5 cm in the LD.

(4) Normal spleen and liver size.

(5) Regression to normal of all nodal non-target disease and disappearance of all detectable.

(6) Non-nodal, non-target disease.

(7) Morphologically negative bone marrow defined as <30% of nucleated cells being lymphoid cells and no lymphoid nodules in a bone marrow sample that is normocellular for age.

(8) Peripheral blood counts meeting all of the following criteria:
(i) ANC >1.5×10⁹/L without need for exogenous growth factors (e.g., G-CSF);
(ii) Platelet count ≥100×10⁹/L without need for exogenous growth factors;
(iii) Hemoglobin ≥110 g/L (11.0 g/dL) without red blood cell transfusions or need for exogenous growth factors (e.g., erythropoietin).

Patients who fulfill all the criteria for a CR (including bone marrow criteria) but who have a persistent anemia, thrombocytopenia, or neutropenia or a hypocellular bone marrow that is related to prior or ongoing drug toxicity (and not to CLL) were considered as a CR with incomplete marrow recovery (CRi).

To satisfy criteria for a partial response (PR), all of the following criteria must have been met:

(1) No evidence of new disease.

(2) Change in disease status meeting ≥2 of the following criteria, with 2 exceptions in which only 1 criterion is needed: 1) only lymphadenopathy is present at baseline; 2) only lymphadenopathy and lymphocytosis are present at baseline. In these 2 cases, only lymphadenopathy must improve to the extent specified below:
(i) In a patient with baseline lymphocytosis (ALC ≥4×10⁹/L), a decrease in peripheral blood ALC by ≥50% from baseline or a decrease to <4×10⁹/L;
(ii) A decrease by ≥50% from the baseline in the SPD of the target nodal lesions;
(iii) In a patient with enlargement of the spleen at baseline, a splenomegaly response as defined above;
(iv) In a patient with enlargement of the liver at baseline, a hepatomegaly response as defined above;
(v) A decrease by ≥50% from baseline in the CLL marrow infiltrate or in B-lymphoid nodules.

(3) No target, splenic, liver, or non-target disease with worsening that meets the criteria for definitive PD.

(4) Peripheral blood counts meeting one of the following criteria:
(i) ANC >1.5×10⁹/L or >50% increase over baseline without need for exogenous growth factors (e.g., G-CSF);
(ii) Platelet count >100×10⁹/L or ≥50% increase over baseline without need for exogenous growth factors;
(iii) Hemoglobin >110 g/L (11.0 g/dL) or ≥50% increase over baseline without red blood cell transfusions or need for exogenous growth factors (e.g., erythropoietin).

To satisfy criteria for stable disease (SD), the following criteria must have been met:

(1) No evidence of new disease.

(2) There is neither sufficient evidence of tumor shrinkage to qualify for PR nor sufficient evidence of tumor growth to qualify for definitive progression of disease (PD).

The occurrence of any of the following events indicates definitive PD:

(1) Evidence of any new disease:
(i) A new node that measures >1.5 cm in the LD and >1.0 cm in the LPD;
(ii) New or recurrent splenomegaly, with a minimum LVD of 14 cm;
(iii) New or recurrent hepatomegaly, with a minimum LVD of 20 cm;
(iv) Unequivocal reappearance of an extra-nodal lesion that had resolved;
(v) A new unequivocal extra-nodal lesion of any size;
(vi) *New non-target disease (e.g., effusions, ascites, or other organ abnormalities related to CLL).

*Isolated new effusions, ascites, or other organ abnormalities are not sufficient evidence alone of PD unless histologically confirmed. Thus, a declaration of PD should not be made if this is the only manifestation of apparently new disease.

(2) Evidence of worsening of target lesions, spleen or liver, or non-target disease:
(i) Increase from the nadir by ≥50% from the nadir in the SPD of target lesions;
(ii) Increase from the nadir by ≥50% in the LD of an individual node or extra-nodal mass that now has an LD of >1.5 cm and an LPD of >1.0 cm;
(iii) Splenic progression, defined as an increase in splenic enlargement by ≥50% from nadir (with a minimum 2 cm increase and a minimum LVD of 14 cm);
(iv) Hepatic progression, defined as an increase in hepatic enlargement by ≥50% from nadir (with a minimum 2 cm increase and minimum LVD of 20 cm);
(v) Unequivocal increase in the size of non-target disease (e.g., effusions, ascites, or other organ abnormalities related to CLL);
(vi) Transformation to a more aggressive histology (e.g., Richter's syndrome) as established by biopsy (with the date of the biopsy being considered the date of CLL progression if the patient has no earlier objective documentation of CLL progression).

(3) Decrease in platelet count or hemoglobin that is attributable to CLL, is not attributable to an autoimmune phenomenon, and is confirmed by bone marrow biopsy showing an infiltrate of clonal CLL cells:
(i) The current platelet count is <100×10⁹/L and there has been a decrease by >50% from the highest on-study platelet count.

(ii) The current hemoglobin is <110 g/L (11.0 g/dL) and there has been a decrease by >20 g/L (2 g/dL) from the highest on-study hemoglobin.

If there was uncertainty regarding whether there was true progression, the patient continued study treatment and remained under close observation pending confirmation of progression status by the IRC. In particular, worsening of constitutional symptoms in the absence of objective evidence of worsening CLL was not considered definitive disease progression; in such patients, both CLL-related and non-CLL-related causes for the constitutional symptoms were considered.

Worsening of disease during temporary interruption of study treatment (e.g., for inter current illness) was not necessarily indicative of resistance to study treatment. In these instances, CT/MRI or other relevant evaluations was considered in order to document whether definitive disease progression had occurred. If subsequent evaluations suggested that the patient had experienced persistent definitive CLL progression, then the date of progression was the time point at which progression was first objectively documented.

In a patient who did not have evidence of PD, the occurrence of any of the following conditions indicated a response status of non-evaluable (NE):
(1) There are no images or inadequate or missing images.
(2) Images of the liver and spleen are missing at that time point (with the exception that absence of splenic images will not result in an NE designation in a patient known to have undergone splenectomy).

Results:

Ten patients were initially treated: 9 with CLL (3 on the 100 mg pembro cohort and 6 in the 200 mg pembro cohort) and 1 with Richter's Transformation (100 mg pembro cohort). This example reports on the 9 CLL pts who were evaluable for safety and efficacy. Baseline demographics were as follows: male/female (5/4), median age 71 years (range 60-81), median prior therapies 1 (1-3), 78% refractory. 56% were treated with a BTK inhibitor (ibrutinib or acalabrutinib) prior to study enrollment, all of which were refractory to BTK therapy. 78% had at least 1 high risk genetic feature (del17p, del11q, TP53 mut, Notchl mut or complex karyotype). All grade and grade ≥3 adverse events (AEs) during cycles 3-6 (umbralisib tosylate, ublituximab, and pembro combination) are listed in Table 7. Of note, only one dose limiting toxicity (DLT) occurred (ALT/AST elevation—200 mg pembro cohort) which triggered expansion to 6 patients, with no additional DLTs reported. The maximum tolerated dose (MTD) was not reached and therefore the primary study endpoint was met. An increase in expected grade ≥3 PI3K-delta-associated toxicities including pneumonitis, colitis, and transaminitis (1 event) was not observed. ORR was 75% for the non-BTK refractory patients (3/4) and 60% in BTK refractory patients (3/5).

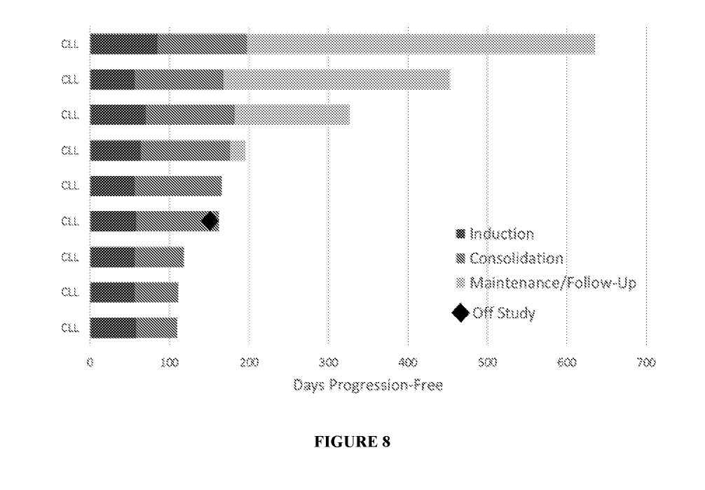

At the time of this analysis, only one patient experienced progressive disease (PD) and no deaths were observed (FIG. 7). Eight of nine patients remain on study in follow up (range 4-21+ months). Of note, patient 1 elected not to participate in the maintenance phase and has achieved an ongoing 21+ month progression free survival (PFS) without therapy. FIG. 8 includes a “swimmers plot” for all 9 CLL study patients. Four patients have had detailed correlative analyses. In one patient, there was a 20-fold increase in bone marrow CLL cells expressing PD-L1/PD-L2 post treatment with pembro. The proportions of the major T cell subsets (including Tregs) and PD1 levels did not change appreciably during therapy.

TABLE 7
All causality adverse events (AEs) during Cycles 3-6 with umbralisib
tosylate + ublituximab + pembro (n = 9)
(All Grades > 20% and all Grade 3/4 AEs)

| | All Grades | All Grades | Grade 3/4 | Grade 3/4 |
|---|---|---|---|---|
| Adverse Event (AE) | (N) | (%) | (N) | (%) |
| Neutropenia | 4 | 44 | 3 | 33 |
| Cough | 4 | 44 | | |
| Leukopenia | 3 | 33 | 1 | 11 |
| Decreased appetite | 3 | 33 | | |
| Anemia | 2 | 22 | 1 | 11 |
| Blood ALK phosphatase | 2 | 22 | | |
| increase | | | | |
| Chills | 2 | 22 | | |
| Fatigue | 2 | 22 | | |
| Headache | 2 | 22 | | |
| Hypothyroidism | 2 | 22 | | |
| Edema | 2 | 22 | | |
| Oropharyngeal pain | 2 | 22 | | |
| Rash | 2 | 22 | | |
| Thrombocytopenia | 2 | 22 | | |
| AST/ALT Increase | 1 | 11 | 1 | 11 |

Conclusion:

This is the first report of a PI3K-δ inhibitor studied in combination with anti-PD-1 therapy in patients with CLL. The triplet combination of umbralisib tosylate+ublituximab+pembro was well-tolerated with durable responses in patients refractory to BTK inhibitor therapy. Enrollment is ongoing in both the CLL and Richter's transformation cohorts.

### Example 2

### Combination of Ublituximab, TGR-1202, and Atezolizumab for Treating Patients with B-cell malignancies

Study Design

In a Phase I/II clinical study, ublituximab, TGR-1202, and atezolizumab are administered to patients with B-cell malignancies (e.g., CLL, NHL), including those with relapsed or refractory disease who require therapy. The study is conducted to determine the safety of TGR-1202+ublituximab+atezolizumab following the combination induction treatment of ublituximab+TGR-1202 in patients with relapsed-refractory B-cell malignancies.

The study also evaluates the clinical efficacy of TGR-1202+ublituximab+atezolizumab following the combination induction treatment of ublituximab+TGR-1202 in patients with a relapsed-refractory B-cell malignancy. Efficacy will be measured as overall response rate, complete response rate, and progression free survival for this cohort.

The dosing schedule and assessment of anti-tumor response are as described in Example 1, except that the anti-PD-L1 antibody, atezolizumab, is administered in the treatment phase, in place of the anti-PD-1 antibody, pembrolizumab. In this regard, while dosages for ublituximab and TGR-1202, in the induction and treatment phases, are the same as described in Example 1, the dosages for the anti-PD-L1 antibody, atezolizumab, in the treatment phase, is as follows:

Dose level 1: Atezolizumab: 600 mg IV infusion every three weeks for four cycles of therapy. Atezolizumab is administered on day 1 of each 21 day cycle.

Dose level 2: Atezolizumab: 1200 mg IV infusion every three weeks for four cycles of therapy. Atezolizumab is administered on day 1 of each 21 day cycle.

### Example 3

### Combination of Ublituximab, TGR-1202, and Atezolizumab (without an Induction Phase) for Treating Patients with B-cell Malignancies

Study Design

In a Phase I/II clinical study, ublituximab, TGR-1202, and atezolizumab are administered to patients with B-cell malignancies (e.g., CLL, NHL, Richter's transformation), including those with relapsed, refractory, or aggressive disease requiring therapy. The study is conducted to determine the safety of the triplet combination of TGR-1202+ublituximab+atezolizumab without the combination induction treatment of ublituximab+TGR-1202 in patients with relapsed-refractory B-cell malignancies.

The study also evaluates the clinical efficacy of TGR-1202+ublituximab+atezolizumab without the combination induction treatment of ublituximab+TGR-1202 in patients with a relapsed-refractory B-cell malignancy. Efficacy will be measured as overall response rate, complete response rate, and progression free survival for this cohort. Assessment of anti-tumor response will be carried out as reported in Example 1.

Since there is no induction phase, the dosing schedule for this triplet combination is as described in the treatment phase of Example 1, except that the anti-PD-L1 antibody, atezolizumab, is administered in place of the anti-PD-1 antibody, pembrolizumab. Also, unlike Example 1, all three agents (TGR-1202+ublituximab+atezolizumab) are administered together on day 1 of cycle 1. The dosages for ublituximab and TGR-1202 are the same as described in Example 1 and the dosages for the anti-PD-L1 antibody, atezolizumab, are as described in Example 2.

### Example 4

### Production of Optimized PD-L1 Antibodies

Antigens were biotinylated using the EZ-Link Sulfo-NHS-Biotinylation Kit from Pierce. Goat anti-human F(ab′)2 kappa-FITC (LC-FITC), Extravidin-PE (EA-PE), and streptavidin-633 (SA-633) were obtained from Southern Biotech, Sigma, and Molecular Probes, respectively. Streptavidin MicroBeads and MACS LC separation columns were purchased from Miltenyi Biotec.

Affinity Maturation

Binding optimization of naive clones was carried out utilizing three maturation strategies: diversification of VH CDRH1/CDRH2, PCR mutagenesis of the VH gene, and VH mutagenesis with a focus on CDRH3.

CTI-07 Lineage

The first cycle of optimization focused on selection of improved binders from a library in which the CTI-07 VH gene was diversified by mutagenic PCR using techniques known in the art. Round 1: Selections were performed by presenting VH mutated forms of the full-length CTI-07 IgG on the surface of yeast. These libraries were incubated with 100 nM biotinylated PD-L1, then detecting IgG expression by an anti-LC FITC reagent (IgG expression) and SA-633 (detection of antigen binding) and viable cells by propidium iodine staining. The top antigen binding/IgG expressing cells were selected by FACS. Round 2: Selections were performed as per Round 1, but using 10 nM biotinylated PD-L1 for discrimination of antigen binding. Round 3: Library expression was carried out as per Rounds 1 and 2. Round 3 employed the use of a poly-specificity reagent (PSR) to remove non-specific antibodies from the selection output (Xu, Y. et.al., PEDS 26:663-670 (2013)). These libraries were incubated with 1:10 dilution of biotinylated PSR reagent, IgG expression was detected by an anti-LC FITC reagent (IgG expression) and PSR binding was detected by EA-PE (detection of antigen binding) and viable cells by propidium iodine staining. The top 1-2% of IgG positive, PSR negative, PI negative cells were sorted and carried to Round 4. Round 4: Selections were performed as per Round 2, but using 1 nM biotinylated PD-L1 for discrimination of antigen binding. Top clones were plated, and sequenced to determine unique IgG sequences. Unique IgG sequences were submitted for antibody production, purification, and characterization.

The second cycle of optimization focused on the selection of improved binders from a library in which the VH gene was diversified by mutagenic PCR while also utilizing degenerate CDRH3 oligos to increase the mutagenic rate within CDRH3. This amplification technique was performed using techniques known in the art. Round 1: Selections were performed by presenting VH mutated forms of the full-length parent IgG on the surface of yeast. These libraries were incubated with 10 nM biotinylated PD-L1, then detecting IgG expression by an anti-LC FITC reagent (IgG expression) and SA-633 (detection of antigen binding) and viable cells by propidium iodine staining. The top antigen binding/IgG expressing cells were selected by FACS. Round 2: Selections were performed as per Round 1, but using 2 nM biotinylated PD-L1 for discrimination of antigen binding Top clones were plated, and sequenced to determine unique IgG sequences. Unique IgG sequences were submitted for antibody production, purification, and characterization.

CTI-09 Lineage

CTI-09 optimization employed the use of CDRH1 and CDRH2 variegation: The CDRH3 of CTI-09 was amplified by PCR and then recombined into a premade vector library with CDRH1 and CDRH2 variants of a diversity of 1×10⁸. Round 1: Selections were performed by presenting VH mutated forms of the full-length CTI-09 IgG on the surface of yeast. These libraries were incubated with 100 nM biotinylated PD-L1. Antigen positive cells were selected by magnetic separation via the Miltenyi MACS system. In short, libraries incubated with b-PD-L1 were incubated with streptavidin magnetic beads. Yeast/bead complexes were captured on a MACS LS column, with unlabeled cells passing into the waste. b-PD-L1 binding cells were then eluted into media for propagation for Round 2 of the selection process. Round 2: Selections were performed by presenting VH mutated forms of the full-length CTI-07 IgG on the surface of yeast. These libraries were incubated with 20 nM biotinylated PD-L1, then detecting IgG expression by an anti-LC FITC reagent (IgG expression) and SA-633 (detection of antigen binding) and viable cells by propidium iodine staining. The top antigen binding/IgG expressing cells were selected by FACS. Round 3: Library expression was carried out as per Rounds 1 and 2. Round 3 employed the use of a poly-specificity reagent (PSR) to remove non-specific antibodies from the selection output (Xu et. al., supra). These libraries were incubated with 1:10 dilution of biotinylated PSR reagent, IgG expression was detected by an anti-LC FITC reagent (IgG expression) and PSR binding was detected by EA-PE (detection of antigen binding) and viable cells by propidium iodine staining. The top 1-2% of IgG positive, PSR negative, PI negative cells were sorted and carried to Round 4. Round 4: The induced Round 3 output was incubated with 20 nM b-PD-L1. Cells were pelleted and washed to remove any remaining b-PD-L1. This cell pellet was resuspended in 1 uM unlabeled PD-L1. Top antigen binders were discriminated by their ability to retain b-PD-L1 antigen over time. Top clones were plated, and sequenced to determine unique IgG sequences. Unique IgG sequences were submitted for antibody production, purification and characterization.

Antibody Production and Purification

Yeast clones were grown to saturation and then induced for 48 hours at 30° C. with shaking. After induction, yeast cells were pelleted and the supernatants harvested for purification. IgGs were purified using a Protein A column and eluted with acetic acid, pH 2.0. Fab fragments were generated by papain digestion and purified over KappaSelect (GE Healthcare LifeSciences).

### Example 5

### Competitive FACS with PD-L1 Antibodies

Chinese hamster ovary (CHO) cells were transfected with a PD-L1 expression vector and subsequently selected for expression of the protein (PD-L1+cells). The CHO cells were incubated with 1 μg/ml biotin-labeled PD-1 for 1 hour.

Following incubation with biotin-labeled PD-1, anti-PD-L1 antibodies were added to the supernatant at 4-fold dilutions, starting at 10 μm/ml, and allowed to incubate for 1 hour. The cells were washed and then contacted with streptavidin-PE. Streptavidin-PE staining was analyzed by flow cytometry to determine percent inhibition of PD-1 binding by the anti-PD-L1 antibodies.

TABLE 8

| | Clinical | | | | |
|---|---|---|---|---|---|
| | Control mAb | CTI-09 | CTI-48 | CTI-50 | CTI-58 |
| IC₅₀, | 9.19e−007 | 4.156e−005 | 9.985e−007 | 1.037e−006 | 3.463e−006 |
| g/ml | | | | | |

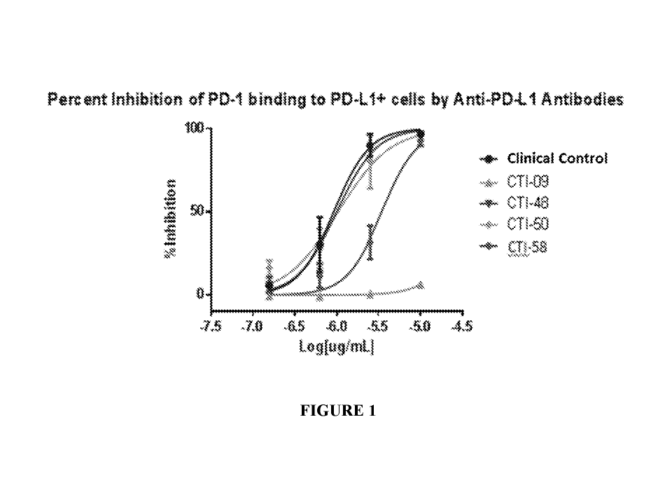

IC₅₀ values for several antibodies, including Clinical Control mAb (as defined by the VH domain represented by SEQ ID NO: 83 and the VL domain represented by SEQ ID NO:84), CTI-09, CTI-48, CTI-50, and CTI-58 were calculated and can be found in Table 8. FIG. 1 shows the results of this study.

### Example 6

### Antibody Binding Kinetics, Specificity, and Selectivity

Octet data analysis was used in determining affinity measurements to assess antibody binding kinetics. 2 mL of the loading sample was prepared at 20 ug/mL (default concentration) in kinetic buffer. Aliquot at least 200 uL into a black 96-well plate. Concentration ranges for the sample were based on the estimated K_{D} of the interaction (if available). Generally, the serial dilution was in a range from 0.1 K_{D} to 10 K_{D}. A 7-point dilution was made into the sample column using kinetic buffer as the sample diluent. The last well of the sample column was used as a reference well later in data analysis, should only contain kinetic buffer.

Biosensors were hydrated in kinetic buffer (lx PBS, 0.1% BSA, 0.02% Tween20, 0.05% Sodium Azide) at room temperature for 10 minutes.

TABLE 9

| | Loading | | | |
|---|---|---|---|---|
| Sample | Sample | | | |
| ID | ID | K_{D} (M) | kon (l/Ms) | kdis (l/s) |
| huPDL1 | CTI-48 | 8.47E−10 | 7.20E+05 | 6.10E−04 |
| msPDL1 | CTI-48 | N/A | N/A | N/A |
| cynoPDL1 | CTI-48 | 5.55E−10 | 1.14E+06 | 6.35E−04 |

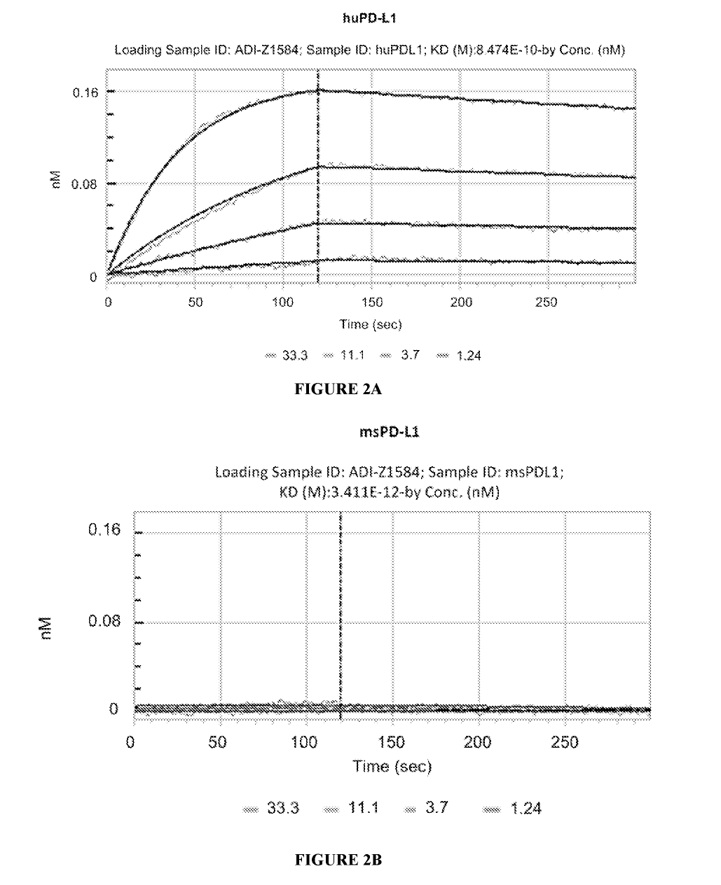

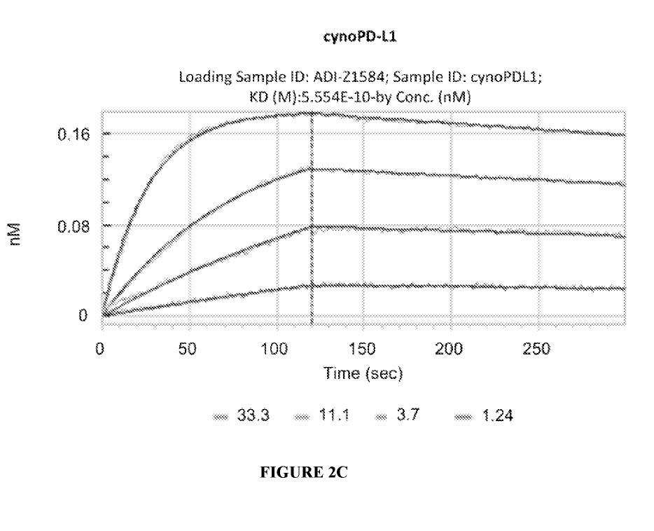

Kinetic values for one exemplary anti-PD-L1 antibody, CTI-48, can be found in Table 9. The experimental results are shown in FIG. 2.

### Example 7

### Antibody Binding Affinity

ForteBio affinity measurements were performed generally as previously described (see, e.g., Estep, P. et al., mAbs 5:270-278 (2013)). Briefly, ForteBio affinity measurements were performed by loading IgGs on-line onto AHQ sensors. Sensors were equilibrated off-line in assay buffer for 30 minutes and then monitored on-line for 60 seconds for baseline establishment. Sensors with loaded IgGs were exposed to 100 nM antigen for 5 minutes; afterwards, they were transferred to assay buffer for 5 minutes for off-rate measurement. Kinetics were analyzed using the 1:1 binding model.

TABLE 10

| | VH CDR3 | IgG K_{D} (M) | | |
|---|---|---|---|---|
| Ab Name | Lineage | Monovalent | kon (1/Ms) | koff (1/s) |
| CTI-09 | 1 | N.B. | | |
| CTI-48 | 1 | 8.24E−10 | 7.68E+05 | 6.33E−04 |
| CTI-49 | 1 | 2.31E−09 | 7.28E+05 | 1.68E−03 |
| CTI-76 | 1 | 8.24E−09 | 6.62E+05 | 5.45E−03 |
| CTI-77 | 1 | 3.25E−09 | 5.44E+05 | 1.77E−03 |
| CTI-78 | 1 | 3.46E−09 | 6.18E+05 | 2.14E−03 |
| CTI-50 | 1 | 1.91E−09 | 7.94E+05 | 1.52E−03 |
| CTI-07 | 2 | 7.97E−08 | 4.92E+05 | 3.92E−02 |
| CTI-58 | 2 | 2.41E−08 | 4.61E+05 | 1.11E−02 |
| Clinical Control | NA | 9.5E−10 | | |
| mAb | | | | |
| CTI-57 | 2 | 8.6E−10 | 5.2E+05 | 4.5E−04 |
| CTI-97 | 1 | 1.82E−09 | 5.11E+05 | 9.28E−04 |
| CTI-98 | 1 | 1.70E−09 | 5.02E+05 | 8.52E−04 |

Binding values for several exemplary PD-L1 antibodies are shown in Table 10. (N.B.=no binding).

### Example 8

### ADCC Activity of Anti-PD-L1 Antibodies

Reporter bioassays were performed in order to determine antibody-dependent cell—mediates cytotoxicity (ADCC) of the disclosed anti-PD-L1 antibodies. The assays utilized SUDHL-1 lymphoma cells and donor PBMCs. Various antibodies were tested at concentrations of 1 or 3 μg/ml.

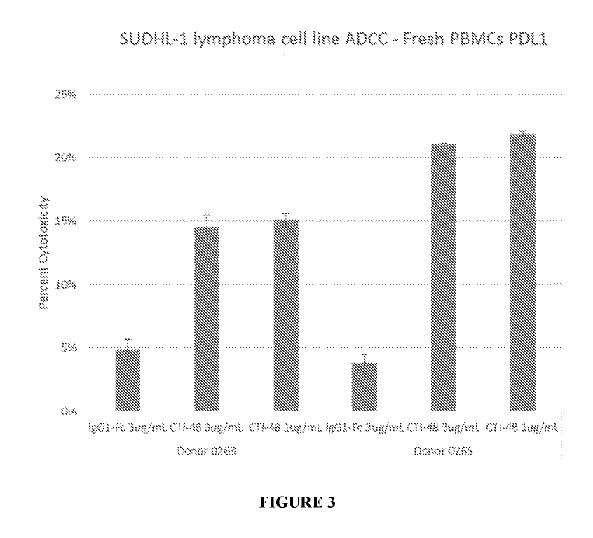

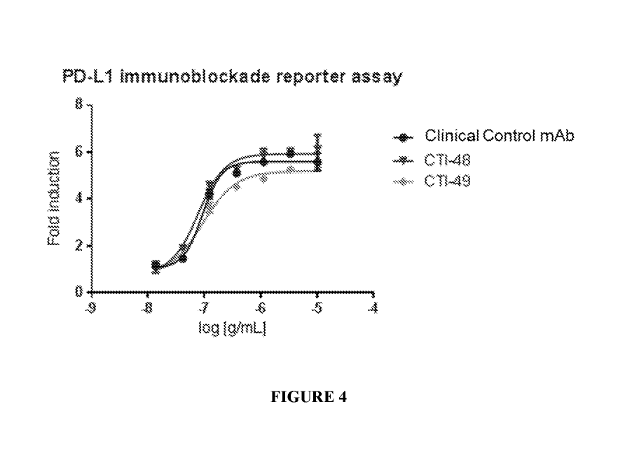

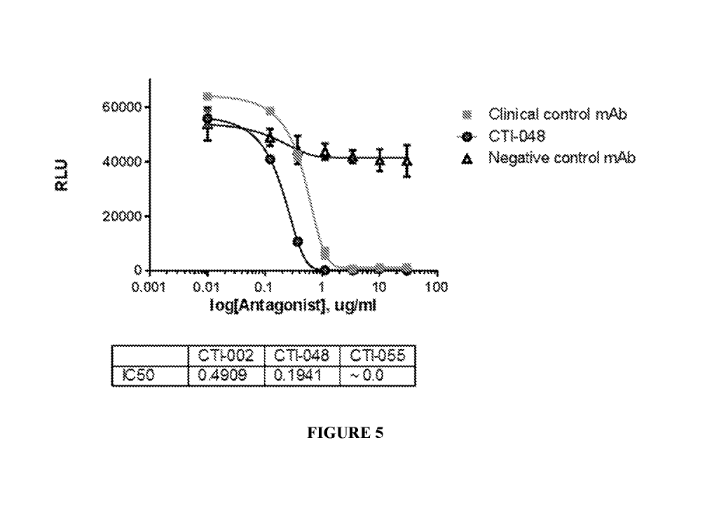

The results of this study, which are shown for exemplary anti-PD-L1 antibody CTI-48 in FIG. 3, are represented by percent cytotoxicity following a 4 hour incubation with the disclosed antibodies.

### Example 9

### Immunoblockade Reporter Assay

Immunoblockade assays were performed using a PD1/PD-L1 Blockade Assay Kit (Promega, CS187111) in 96 well plates. Three major events occur in the assay. Event 1: TCR-mediated NFAT activation occurs when engineered Jurkat PD-1 Effector cells and aAPC (artificial antigen presenting cell) PD-L1 cells are engaged through TCR/TCR activator interaction. Event 2: Inhibition of NFAT signal by PD-1:PD-L1 ligation when no blocking antibodies are present. Event 3: Recovery of NFAT signal by addition of anti-PD-1 or anti-PD-L1 blocking antibody.

The day before assay, 25 mL of cell recovery medium (10% FBS/F-12) was made in 50 mL conical tubes for Thaw-and-Use PD-L1 cells by adding 2.5 mL FBS to 22.5 mL F-12. One vial of Thaw-and-Use PD-L1 cells (CS187103) was removed from freezer storage and transferred to the bench on dry ice. The vial was thawed in a 37° C. water bath until cells were just thawed (about 3-4 minutes). The cell suspension was gently mixed in the vial by pipetting up and down. All the cells (0.5 mL) were transferred to a tube labeled “PD-L1 cells” containing 14.5 mL cell recovery medium, followed by gentle inversions. The cell suspension was transferred to a sterile reagent reservoir. Immediately, using a multichannel pipette, 100 μL of cell recovery medium was added per well to outside wells for assay plates. The plates were incubated overnight in a CO₂ incubator at 37° C.

On the day of assay, fresh assay buffer (RPMI 1640+1% FBS) was prepared, and seven-point three-fold serial dilutions were made in assay buffer for each of the test antibodies at 2× of final concentration. 95 μL of medium was removed from all the wells on the assay plates, and 40 μL of serial dilutions of the test antibodies was added to the wells containing PD-L1 cells. 80 μL per well assay buffer was added to the outside wells for each plate.

Thaw-and-Use PD-1 Effector cells (CS187105) were transferred into the assay plates, and the plates were incubated for six hours at 37° C. in CO₂ incubator. After the six-hour induction, the assay plates were removed from the CO₂ incubator and equilibrated at ambient temperature for 5-10 minutes. 80 μL of Bio-Glo™ Reagent was added to every test well, and the plates were incubated for another 5-10 minutes at ambient temperature. Luminescence was measured in a POLARstar Omega plate reader with 0.5 second integration.

The following antibodies were tested with final concentrations of 10 μg/mL, 3.33 μg/mL, 1.11 μg/mL, 0.37 μg/mL, 0.123 μg/mL, 0.041 μg/mL, and 0.014 μg/mL: CTI-2, Clinical Control mAb, CTI-09, CTI-48, CTI-50, CTI-07, and CTI-58.

Results for exemplary PD-L1 antibodies, including Clinical Control mAb, CTI-48, and CTI-49, are shown in Table 11 below.

TABLE 11

| | Clinical | | |
|---|---|---|---|
| | Control mAb | CTI-48 | CTI-49 |
| EC₅₀, g/ml | 9.213e−008 | 7.750e−008 | 9.191e−008 |

### Example 10

### PD-L1/B7.1 Inhibitor Screening Assay

A commercially available assay kit was used to screen and profile the interaction of the disclosed antibodies and the PD-L1/B7.1 interaction. The kit came in a 96-well format with biotin-labeled B7-1 (CD80), purified PD-L1, streptavidin-labeled HRP, and assay buffer for 100 binding reactions. The kit was used to detect biotin-labeled B7.1 by streptavidin-HRP.

First, PD-L1 was coated on a 96-well plate. Next, either one of the disclosed antibodies, a positive control, a substrate control, or a blank was added to each well and incubated prior to the addition of B7.1-biotin. Finally, the plate was treated with streptavidin-HRP followed by addition of an HRP substrate to produce chemiluminescence, which can then be measured by a chemiluminescence reader.

The following antibodies were tested for their inhibitory effect on the binding of PD-L1 and B7.1 at concentrations of 30 μg/mL, 10 μg/mL, 3.33 μg/mL, 1.11 μg/mL, 0.37 μg/mL, and 0.123 μg/mL: CTI-1, CTI-2, CTI-33, CTI-48, and CTI-55.

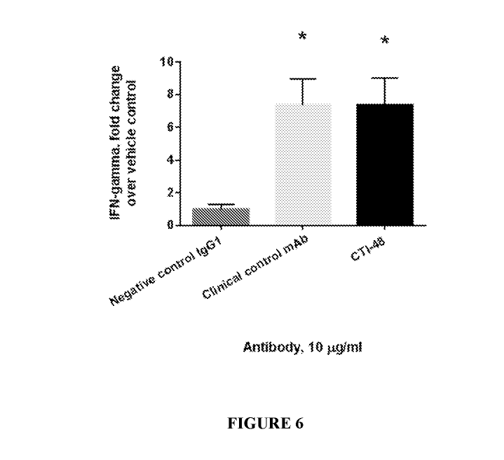

Exemplary results indicate that the IC₅₀ for binding inhibition of the disclosed antibodies ranged between 0.1816 and 0.5056 μg/mL. For instance, the IC₅₀ of CTI-48 was calculated to be 0.1816 μg/mL. A comparison of the activity of CTI-48 and a clinical control mAb is shown in FIG. 5.

### Example 11

### Effect of PD-L1 Antibodies on IFN-γ Production

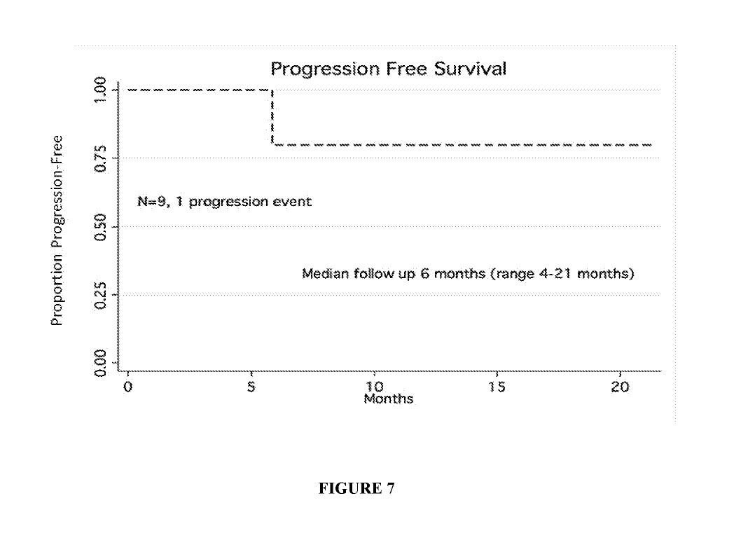

Antibodies were dosed into mixed lymphocyte reaction (MLR) cultures in order to determine the effects of the disclosed antibodies on IFN-γ production. The fold change in production of IFN-γ was determined after a 4-day MLR culture with antibodies at a concentration of 10 μg/mL. Exemplary results, including those of an appropriate isotype control (hIgG1), are shown in FIG. 6. As shown in FIG. 6, CTI-48 induced a comparable response to a clinical control mAb. Further, many of the tested PD-L1 antibodies elicited a statistically significant increase in IFN-γ production, including a roughly 10-fold increase by CTI-33 and CTI-55 over control levels.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

All patents and publications mentioned in the specification are indicative of the levels of those of ordinary skill in the art to which the disclosure pertains. All patents and publications are herein incorporated by reference to the same extent as if each individual publication was specifically and individually indicated to be incorporated by reference.

What is claimed is:

1. A method for treating a subject afflicted with chronic lymphocytic leukemia (CLL) comprising administering to the subject in a treatment phase:
(i) a therapeutically effective amount of a PI3-kinase delta inhibitor, wherein the PI3-kinase delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate, or prodrug thereof;
(ii) a therapeutically effective amount of ublituximab; and
(iii) a therapeutically effective amount of an anti-PD-1 antibody.

2. The method of claim 1, wherein the anti-PD-1 antibody is pembrolizumab.

3. The method of claim 1 or 2, wherein the PI3-kinase delta inhibitor is in the form of a p-toluenesulfonic acid (PTSA) salt.

4. The method of claim 2 or 3, wherein the PI3-kinase delta inhibitor, the ublituximab, and the pembrolizumab are administered to the subject simultaneously, sequentially, or both simultaneously and sequentially.

5. The method of any one of claim 1, 2, 3, or 4, wherein the PI3-kinase delta inhibitor is administered daily at a dose from: about 200 to about 1200 mg, about 400 to about 1000 mg, about 400 to about 800 mg, about 600 mg, about 700 mg, about 800 mg, about 900 mg, about 1000 mg, or about 1200 mg.

6. The method of claim 5, wherein the PI3-kinase delta inhibitor is administered daily at a dose of about 800 mg.

7. The method of any one of claims 1-6, wherein the PI3-kinase delta inhibitor is micronized.

8. The method of claim 7, wherein the PI3-kinase delta inhibitor is formulated for oral administration.

9. The method of any one of claims 1-8, wherein ublituximab is administered at a dose from: about 450 to about 1200 mg, about 450 to about 1000 mg, about 500 to about 1200 mg, about 500 to about 1000 mg, about 500 to about 900 mg, about 600 to about 1200 mg, about 600 to about 1000 mg, about 600 to about 900 mg, about 500 mg, about 600 mg, about 700 mg, about 750 mg, about 800 mg, about 900 mg, about 1000 mg, about 1100 mg, or about 1200 mg, about once every 4 to 7 weeks, about once every 5 to 7 weeks, about once every 5 to 6 weeks, about once a week, about once every 2 weeks, about once every 3 weeks, about once every 4 weeks, about once every 5 weeks, about once every 6 weeks, or about once every 7 weeks.

10. The method of claim 9, wherein ublituximab is administered at a dose of about 900 mg about once every 6 weeks.

11. The method of claim 9 or 10, wherein the first dose of ublituximab is administered on day 1 of the sixth week after the treatment phase is initiated.

12. The method of any one of claims 1-11, wherein ublituximab is formulated for intravenous infusion.

13. The method of any one of claims 1-12, wherein pembrolizumab is administered at a dose from: about 100 to about 300 mg, about 100 to about 200 mg, about 100 mg, about 150 mg, about 200 mg, or about 250 mg about once every 2 to 4 weeks, or about once every 3 to 4 weeks, or about once every 3 weeks.

14. The method of claim 13, wherein pembrolizumab is administered at a dose of about 100 mg or 200 mg about once every 3 weeks.

15. The method of claim 13 or 14, wherein the first dose of pembrolizumab is administered at a dose of about 100 mg.

16. The method of any one of claims 1-15, wherein pembrolizumab is formulated for intravenous infusion.

17. The method of any one of claims 1-16, wherein the duration of the treatment phase is up to about 15 weeks, up to about 14 weeks, up to about 13 weeks, or up to about 12 weeks.

18. The method of claim 17, wherein the duration of the treatment phase is about 12 weeks.

19. The method of any one of claims 1-18, further comprising, prior to the treatment phase, an induction phase, comprising administering to the subject:
(i) a therapeutically effective amount of a PI3-kinase delta inhibitor, wherein the PI3-kinase delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate or prodrug thereof; and
(ii) a therapeutically effective amount of ublituximab.

20. The method of claim 19, wherein the PI3-kinase delta inhibitor is in the form of a p-toluenesulfonic acid (PTSA) salt.

21. The method of claim 19 or 20, wherein the PI3-kinase delta inhibitor and the ublituximab are administered to the subject simultaneously, sequentially or both simultaneously and sequentially during the induction phase.

22. The method of any one of claims 19-21, wherein the PI3-kinase delta inhibitor is administered daily at a dose from: about 200 to about 1200 mg, about 400 to about 1000 mg, about 400 to about 800 mg, about 600 mg, about 700 mg, about 800 mg, about 900 mg, about 1000 mg, or about 1200 mg during the induction phase.

23. The method of claim 22, wherein the PI3-kinase delta inhibitor is administered daily at a dose of about 800 mg during the induction phase.

24. The method of any one of claims 19-23, wherein the PI3-kinase delta inhibitor is micronized.

25. The method of claim 24, wherein the PI3-kinase delta inhibitor is formulated for oral administration during the induction phase.

26. The method of any one of claims 19-25, wherein ublituximab is administered at a dose from: about 450 to about 1200 mg, about 450 to about 1000 mg, about 600 to about 1200 mg, about 600 to about 1000 mg, about 600 to about 900 mg, about 600 mg, about 700 mg, about 800 mg, or about 900 mg about once every 1 to 3 weeks, about once every 1 to 2 weeks, about once every 1 week, or about once every 2 weeks during the induction phase.

27. The method of claim 26, wherein ublituximab is administered at a dose of about 900 mg about once every 1 or 2 weeks during the induction phase.

28. The method of any one of claims 19-27, wherein ublituximab is formulated for intravenous infusion during the induction phase.

29. The method of any one of claims 19-28, wherein the first dose of ublituximab is administered on day 1 of the induction phase.

30. The method of claim 29, wherein the first dose of ublituximab, during the induction phase, is divided into 2 or 3 sub-doses to be administered in 2 or 3 consecutive days during the induction phase, or is divided into 2 sub-doses to be administered in 2 consecutive days.

31. The method of claim 30, wherein the first sub-dose of ublituximab comprises up to 150 mg of ublituximab.

32. The method of claim 30 or 31, wherein the second sub-dose of ublituximab comprises up to 750 mg of ublituximab.

33. The method of any one of claims 19-32, wherein the duration of the induction phase is up to about 12 weeks, up to about 11 weeks, up to about 10 weeks, up to about 9 weeks, or up to about 8 weeks.

34. The method of claim 33, wherein the duration of the induction phase is about 8 weeks.

35. The method of any one of claims 1-34, further comprising, after the treatment phase, a maintenance phase, which comprises administering to the subject a therapeutically effective amount of the PI3-kinase delta inhibitor, or a pharmaceutically acceptable salt, solvate or prodrug thereof.

36. The method of claim 35, wherein the PI3-kinase delta inhibitor is administered daily at a dose from: about 200 to about 1200 mg, about 400 to about 1000 mg, about 400 to about 800 mg, about 600 mg, about 700 mg, about 800 mg, about 900 mg, about 1000 mg, or about 1200 mg during the maintenance phase.

37. The method of claim 36, wherein the PI3-kinase delta inhibitor is administered daily at a dose of about 800 mg during the maintenance phase.

38. The method of claim 36 or 37, wherein the PI3-kinase delta inhibitor is micronized.

39. The method of claim 38, wherein the PI3-kinase delta inhibitor is formulated for oral administration during the maintenance phase.

40. The method of any one of claims 35-39, wherein the duration of the maintenance phase is as long as clinical benefit is observed, or until unmanageable toxicity or disease progression occurs.

41. The method of claim 40, wherein the maintenance phase ends when disease progression occurs.

42. The method of claim 40 or 41, wherein the duration of the maintenance phase is at least 3 weeks.

43. The method of any one of claims 1-42, wherein the subject is afflicted with relapsed-refractory CLL.

44. A method for treating a subject afflicted with relapsed-refractory chronic lymphocytic leukemia (CLL) comprising administering to the subject during a treatment phase:
(i) a daily amount of about 800 mg of a PI3-kinase delta inhibitor, wherein the PI3-kinase delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate, or prodrug thereof;
(ii) about 900 mg of ublituximab once every 6 weeks, wherein the first dose of ublituximab is administered on day 1 of the sixth week after the treatment phase is initiated; and
(iii) about 100 mg or 200 mg of pembrolizumab once every 3 weeks, wherein the first dose of pembrolizumab is administered on day 1 when the treatment phase is initiated;
wherein the duration of the treatment phase is about 12 weeks; and
wherein the PI3-kinase delta inhibitor, the ublituximab, and the pembrolizumab are administered to the subject simultaneously, sequentially, or both simultaneously and sequentially.

45. The method of claim 44, further comprising, prior to the treatment phase, an induction phase, comprising administering to the subject:
(i) a daily amount of about 800 mg of a PI3-kinase delta inhibitor, wherein the PI3-kinase delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate, or prodrug thereof; and
(ii) about 900 mg of ublituximab once every 1 or 2 weeks; wherein the first dose of ublituximab is administered on day 1 of the induction phase;
wherein the duration of the induction phase is about 8 weeks; and
wherein the PI3-kinase delta inhibitor and the ublituximab are administered to the subject simultaneously, sequentially or both simultaneously and sequentially.

46. The method of claim 45, wherein the first dose of ublituximab is divided into 2 sub-doses during the induction phase, wherein the first sub-dose comprises up to 150 mg of ublituximab; and the second sub-dose comprises up to 750 mg of ublituximab; and wherein the first and second sub-doses are administered on day 1 and day 2 of the induction phase, respectively.

47. The method of any one of claims 44-46, further comprising, after the treatment phase, a maintenance phase, comprising administering to the subject daily about 800 mg of a PI3-kinase delta inhibitor, or a pharmaceutically acceptable salt, solvate or prodrug thereof, wherein the duration of the maintenance phase is at least 3 weeks.

48. The method of any one of claims 44-47, wherein the PI3-kinase delta inhibitor is micronized and is formulated for oral administration.

49. The method of any one of claims 44-48, wherein the ublituximab and the pembrolizumab are formulated for intravenous infusion.

50. A kit for treating a subject afflicted with relapsed-refractory CLL, the kit comprising:
(i) a single dose or multiple doses of ublituximab;
(ii) a single dose or multiple doses of a PI3-kinase delta inhibitor, wherein the PI3-kinase delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate, or prodrug thereof;
(iii) a single dose or multiple doses of an anti-PD-1 antibody; and
(iv) instructions for using said ublituximab, said PI3-kinase delta inhibitor, and said anti-PD-1 antibody according to the method of one of claims 1-49.

51. The kit of claim 50, wherein said anti-PD-1 antibody is pembrolizumab.

52. A method of treating a subject afflicted with a hematologic cancer, comprising administering to the subject in a treatment phase:
(i) a therapeutically effective amount of a PI3 kinase-delta inhibitor;
(ii) a therapeutically effective amount of an anti-CD20 antibody; and
(iii) a therapeutically effective amount of an anti-PD-1 or anti-PD-L1 antibody.

53. The method of claim 52, wherein the PI3 kinase-delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate, or prodrug thereof.

54. The method of claim 53, wherein the PI3 kinase-delta inhibitor is in the form of a p-toluenesulfonic acid (PTSA) salt.

55. The method of any one of claims 52-54, wherein the anti-CD20 antibody is ublituximab or an antibody fragment that binds the same epitope as ublituximab.

56. The method of any one of claims 52-55, wherein the anti-PD-1 antibody is nivolumab, pembrolizumab, or pidilizumab.

57. The method of any one of claims 52-55, wherein the anti-PD-L1 antibody is CTI-07, CTI-09, CTI-48, CTI-49, CTI-50, CTI-76, CTI-77, CTI-78, CTI-57, CTI-58, CTI-97, CTI-98, CTI-92, CTI-95, CTI-93, CTI-94, CTI-96, durvalumab, BMS-936559, atezolizumab, or avelumab.

58. The method of any one of claims 52-57, wherein the hematological cancer is lymphoma, leukemia, or myeloma.

59. The method of claim 58, wherein the hematological cancer is acute lymphocytic leukemia (ALL), acute myeloid leukemia (AML), chronic lymphocytic leukemia (CLL), small lymphocytic lymphoma (SLL), multiple myeloma (MM), non-Hodgkin's lymphoma (NHL), mantle cell lymphoma (MCL), follicular lymphoma (FL), Waldenstrom's macroglobulinemia (WM), diffuse large B-cell lymphoma (DLBCL), marginal zone lymphoma (MZL), hairy cell leukemia (HCL), Burkitt's lymphoma (BL), or Richter's transformation.

60. The method of claim 58 or 59, wherein the hematological cancer expresses PD-1 or PD-L1.

61. The method of any one of claims 58-60, wherein the hematological cancer is relapsed-refractory disease.

62. The method of any one of claims 58-61, wherein the hematological cancer is relapsed-refractory CLL.

63. A method of treating a subject afflicted with a hematologic cancer, comprising administering to the subject in a treatment phase:
(i) a therapeutically effective amount of a PI3 kinase-delta inhibitor, wherein said PI3 kinase-delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate, or prodrug thereof;
(ii) a therapeutically effective amount of ublituximab; and
(iii) a therapeutically effective amount of an anti-PD-1 antibody or anti-PD-L1 antibody.

64. The method of claim 63, wherein the anti-PD-1 antibody is nivolumab, pembrolizumab, or pidilizumab.

65. The method of claim 63, wherein the anti-PD-L1 antibody is CTI-07, CTI-09, CTI-48, CTI-49, CTI-50, CTI-76, CTI-77, CTI-78, CTI-57, CTI-58, CTI-97, CTI-98, CTI-92, CTI-95, CTI-93, CTI-94, CTI-96, durvalumab, BMS-936559, atezolizumab, or avelumab.

66. The method of any of claim 1-49 or 52-65, wherein the subject is a human.

67. The method of any of claims 52-66, further comprising, prior to the treatment phase, an induction phase, comprising administering to the subject:
(i) a therapeutically effective amount of a PI3 kinase-delta inhibitor, wherein said PI3 kinase-delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate or prodrug thereof; and
(ii) a therapeutically effective amount of ublituximab.

68. The method of claim 67, wherein the PI3 kinase-delta inhibitor is in the form of a p-toluenesulfonic acid (PTSA) salt.

69. The method of any one of claims 52-68, further comprising, after the treatment phase, a maintenance phase, comprising administering to the subject a therapeutically effective amount of a PI3 kinase-delta inhibitor, or a pharmaceutically acceptable salt, solvate or prodrug thereof.

70. The method of any one of claims 52-69, wherein the PI3 kinase-delta inhibitor, the ublituximab, and the anti-PD-1 antibody or anti-PD-L1 antibody are administered to the subject simultaneously, sequentially, or both simultaneously and sequentially.

71. The method of any one of claim 1-49 or 52-70, wherein the PI3 kinase-delta inhibitor is TGR-1202 (umbralisib tosylate).

72. The method of any one of claim 52-55, 57-63, or 65-71, wherein the anti-PD-L1 antibody is CTI-48.

73. A kit for treating a subject afflicted with a hematological cancer, the kit comprising:
(i) a single dose or multiple doses of ublituximab;
(ii) a single dose or multiple doses of a PI3 kinase-delta inhibitor, wherein said PI3 kinase-delta inhibitor is (S)-2-(1-(4-amino-3-(3-fluoro-4-isopropoxyphenyl)-1H-pyrazolo [3,4-d]pyrim-idin-1-yl)ethyl-6-fluoro-3-(3-fluorophenyl)-4H-chromen-4-one, or a pharmaceutically acceptable salt, solvate, or prodrug thereof;
(iii) a single dose or multiple doses of an anti-PD-1 or anti-PD-L1 antibody; and
(iv) instructions for using said ublituximab, said PI3 kinase-delta inhibitor, and said anti-PD-1 or anti-PD-L1 antibody, according to the method of any one of claims 52-71.

74. The kit of claim 73, wherein said anti-PD-1 antibody is pembrolizumab.

75. The kit of claim 73, wherein said anti-PD-L1 antibody is atezolizumab.

76. The kit of any one of claims 73-75, wherein said PI3 kinase-delta inhibitor is in the form of a p-toluenesulfonic acid (PTSA) salt.

77. The kit of any one of claim 50-51 or 73-76, wherein the PI3-kinase delta inhibitor is TGR-1202 (umbralisib tosylate).

78. A PI3-kinase delta inhibitor and/or ublituximab for use in a method for treating a subject afflicted with chronic lymphocytic leukemia (CLL) comprising administering to the subject in a treatment phase:
(i) a therapeutically effective amount of the PI3-kinase delta inhibitor, wherein the PI3-kinase delta inhibitor is (S)-2-(1-(4-amino-3-(3-fluoro-4-isopropoxyphenyl)-1H-pyrazolo [3,4-d]pyrim-idin-1-yl)ethyl-6-fluoro-3-(3-fluorophenyl)-4H-chromen-4-one, or a pharmaceutically acceptable salt, solvate, or prodrug thereof;
(ii) a therapeutically effective amount of ublituximab; and
(iii) a therapeutically effective amount of an anti-PD-1 antibody.

79. A PI3-kinase delta inhibitor and/or ublituximab for use according to claim 78, wherein the anti-PD-1 antibody is pembrolizumab.

80. A PI3-kinase delta inhibitor and/or ublituximab for use according toclaim 78 or 79, wherein the PI3-kinase delta inhibitor is in the form of a p-toluenesulfonic acid (PTSA) salt.

81. A PI3-kinase delta inhibitor and/or ublituximab for use according to claim 79 or 80, wherein the PI3-kinase delta inhibitor, the ublituximab, and the pembrolizumab are administered to the subject simultaneously, sequentially, or both simultaneously and sequentially.

82. A PI3-kinase delta inhibitor and/or ublituximab for use according toany one of claim 78, 79, 80, or 81 wherein the PI3-kinase delta inhibitor is administered daily at a dose from: about 200 to about 1200 mg, about 400 to about 1000 mg, about 400 to about 800 mg, about 600 mg, about 700 mg, about 800 mg, about 900 mg, about 1000 mg, or about 1200 mg.

83. A PI3-kinase delta inhibitor and/or ublituximab for use according toclaim 82, wherein the PI3-kinase delta inhibitor is administered daily at a dose of about 800 mg.

84. A PI3-kinase delta inhibitor and/or ublituximab for use according toclaims 78-83, wherein the PI3-kinase delta inhibitor is micronized.

85. A PI3-kinase delta inhibitor and/or ublituximab for use according toclaim 84, wherein the PI3-kinase delta inhibitor is formulated for oral administration.

86. A PI3-kinase delta inhibitor and/or ublituximab for use according toany one of claims 78-85, wherein ublituximab is administered at a dose from: about 450 to about 1200 mg, about 450 to about 1000 mg, about 500 to about 1200 mg, about 500 to about 1000 mg, about 500 to about 900 mg, about 600 to about 1200 mg, about 600 to about 1000 mg, about 600 to about 900 mg, about 500 mg, about 600 mg, about 700 mg, about 750 mg, about 800 mg, about 900 mg, about 1000 mg, about 1100 mg, or about 1200 mg, about once every 4 to 7 weeks, about once every 5 to 7 weeks, about once every 5 to 6 weeks, about once a week, about once every 2 weeks, about once every 3 weeks, about once every 4 weeks, about once every 5 weeks, about once every 6 weeks, or about once every 7 weeks.

87. A PI3-kinase delta inhibitor and/or ublituximab for use according toclaim 86, wherein ublituximab is administered at a dose of about 900 mg about once every 6 weeks.

88. A PI3-kinase delta inhibitor and/or ublituximab for use according toclaim 86 or 87, wherein the first dose of ublituximab is administered on day 1 of the sixth week after the treatment phase is initiated.

89. A PI3-kinase delta inhibitor and/or ublituximab for use according to any one of claims 78-88, wherein ublituximab is formulated for intravenous infusion.

90. A PI3-kinase delta inhibitor and/or ublituximab for use according to any one of claims 78-89, wherein pembrolizumab is administered at a dose from: about 100 to about 300 mg, about 100 to about 200 mg, about 100 mg, about 150 mg, about 200 mg, or about 250 mg about once every 2 to 4 weeks, or about once every 3 to 4 weeks, or about once every 3 weeks.

91. A PI3-kinase delta inhibitor and/or ublituximab for use according to claim 90, wherein pembrolizumab is administered at a dose of about 100 mg or 200 mg about once every 3 weeks.

92. A PI3-kinase delta inhibitor and/or ublituximab for use according to claim 90 or 91, wherein the first dose of pembrolizumab is administered at a dose of about 100 mg.

93. A PI3-kinase delta inhibitor and/or ublituximab for use according to any one of claims 78-92, wherein pembrolizumab is formulated for intravenous infusion.

94. A PI3-kinase delta inhibitor and/or ublituximab for use according to any one of claims 78-93, wherein the duration of the treatment phase is up to about 15 weeks, up to about 14 weeks, up to about 13 weeks, or up to about 12 weeks.

95. A PI3-kinase delta inhibitor and/or ublituximab for use according to claim 94, wherein the duration of the treatment phase is about 12 weeks.

96. A PI3-kinase delta inhibitor and/or ublituximab for use according to any one of claims 78-95, further comprising, prior to the treatment phase, an induction phase, comprising administering to the subject:
(i) a therapeutically effective amount of the PI3-kinase delta inhibitor, wherein the PI3-kinase delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate or prodrug thereof; and
(ii) a therapeutically effective amount of ublituximab.

97. A PI3-kinase delta inhibitor and/or ublituximab for use according to claim 96, wherein the PI3-kinase delta inhibitor is in the form of a p-toluenesulfonic acid (PTSA) salt.

98. A PI3-kinase delta inhibitor and/or ublituximab for use according to claim 96 or 97, wherein the PI3-kinase delta inhibitor and the ublituximab are administered to the subject simultaneously, sequentially or both simultaneously and sequentially during the induction phase.

99. A PI3-kinase delta inhibitor and/or ublituximab for use according to any one of claims 96-98, wherein the PI3-kinase delta inhibitor is administered daily at a dose from: about 200 to about 1200 mg, about 400 to about 1000 mg, about 400 to about 800 mg, about 600 mg, about 700 mg, about 800 mg, about 900 mg, about 1000 mg, or about 1200 mg during the induction phase.

100. A PI3-kinase delta inhibitor and/or ublituximab for use according to claim 99, wherein the PI3-kinase delta inhibitor is administered daily at a dose of about 800 mg during the induction phase.

101. A PI3-kinase delta inhibitor and/or ublituximab for use according to any one of claims 96-100, wherein the PI3-kinase delta inhibitor is micronized.

102. A PI3-kinase delta inhibitor and/or ublituximab for use according to claim 101, wherein the PI3-kinase delta inhibitor is formulated for oral administration during the induction phase.

103. A PI3-kinase delta inhibitor and/or ublituximab for use according to any one of claims 96-102, wherein ublituximab is administered at a dose from: about 450 to about 1200 mg, about 450 to about 1000 mg, about 600 to about 1200 mg, about 600 to about 1000 mg, about 600 to about 900 mg, about 600 mg, about 700 mg, about 800 mg, or about 900 mg about once every 1 to 3 weeks, about once every 1 to 2 weeks, about once every 1 week, or about once every 2 weeks during the induction phase.

104. A PI3-kinase delta inhibitor and/or ublituximab for use according to claim 103, wherein ublituximab is administered at a dose of about 900 mg about once every 1 or 2 weeks during the induction phase.

105. A PI3-kinase delta inhibitor and/or ublituximab for use according to any one of claims 96-104, wherein ublituximab is formulated for intravenous infusion during the induction phase.

106. A PI3-kinase delta inhibitor and/or ublituximab for use according to any one of claims 96-105, wherein the first dose of ublituximab is administered on day 1 of the induction phase.

107. A PI3-kinase delta inhibitor and/or ublituximab for use according to claim 106, wherein the first dose of ublituximab, during the induction phase, is divided into 2 or 3 sub-doses to be administered in 2 or 3 consecutive days during the induction phase, or is divided into 2 sub-doses to be administered in 2 consecutive days.

108. A PI3-kinase delta inhibitor and/or ublituximab for use according to claim 107, wherein the first sub-dose of ublituximab comprises up to 150 mg of ublituximab.

109. A PI3-kinase delta inhibitor and/or ublituximab for use according to claim 107 or 106, wherein the second sub-dose of ublituximab comprises up to 750 mg of ublituximab.

110. A PI3-kinase delta inhibitor and/or ublituximab for use according to any one of claims 96-109, wherein the duration of the induction phase is up to about 12 weeks, up to about 11 weeks, up to about 10 weeks, up to about 9 weeks, or up to about 8 weeks.

111. A PI3-kinase delta inhibitor and/or ublituximab for use according to claim 110, wherein the duration of the induction phase is about 8 weeks.

112. A PI3-kinase delta inhibitor and/or ublituximab for use according to any one of claims 78-111, further comprising, after the treatment phase, a maintenance phase, which comprises administering to the subject a therapeutically effective amount of the PI3-kinase delta inhibitor, or a pharmaceutically acceptable salt, solvate or prodrug thereof.

113. A PI3-kinase delta inhibitor and/or ublituximab for use according to claim 112, wherein the PI3-kinase delta inhibitor is administered daily at a dose from: about 200 to about 1200 mg, about 400 to about 1000 mg, about 400 to about 800 mg, about 600 mg, about 700 mg, about 800 mg, about 900 mg, about 1000 mg, or about 1200 mg during the maintenance phase.

114. A PI3-kinase delta inhibitor and/or ublituximab for use according to claim 113, wherein the PI3-kinase delta inhibitor is administered daily at a dose of about 800 mg during the maintenance phase.

115. A PI3-kinase delta inhibitor and/or ublituximab for use according to claim 113 or 114, wherein the PI3-kinase delta inhibitor is micronized.

116. A PI3-kinase delta inhibitor and/or ublituximab for use according to claim 115, wherein the PI3-kinase delta inhibitor is formulated for oral administration during the maintenance phase.

117. A PI3-kinase delta inhibitor and/or ublituximab for use according to any one of claims 112-120, wherein the duration of the maintenance phase is as long as clinical benefit is observed, or until unmanageable toxicity or disease progression occurs.

118. A PI3-kinase delta inhibitor and/or ublituximab for use according to claim 117, wherein the maintenance phase ends when disease progression occurs.

119. A PI3-kinase delta inhibitor and/or ublituximab for use according to claim 117 or 118, wherein the duration of the maintenance phase is at least 3 weeks.

120. A PI3-kinase delta inhibitor and/or ublituximab for use according to any one of claims 78-119, wherein the subject is afflicted with relapsed-refractory CLL.

121. A PI3-kinase delta inhibitor and/or ublituximab for use in a method for treating a subject afflicted with relapsed-refractory chronic lymphocytic leukemia (CLL) comprising administering to the subject during a treatment phase:
(i) a daily amount of about 800 mg of the PI3-kinase delta inhibitor, wherein the PI3-kinase delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate, or prodrug thereof;
(ii) about 900 mg of ublituximab once every 6 weeks, wherein the first dose of ublituximab is administered on day 1 of the sixth week after the treatment phase is initiated; and
(iii) about 100 mg or 200 mg of pembrolizumab once every 3 weeks, wherein the first dose of pembrolizumab is administered on day 1 when the treatment phase is initiated;
wherein the duration of the treatment phase is about 12 weeks; and
wherein the PI3-kinase delta inhibitor, the ublituximab, and the pembrolizumab are administered to the subject simultaneously, sequentially, or both simultaneously and sequentially.

122. A PI3-kinase delta inhibitor and/or ublituximab for use according to claim 121, further comprising, prior to the treatment phase, an induction phase, comprising administering to the subject:
(i) a daily amount of about 800 mg of the PI3-kinase delta inhibitor, wherein the PI3-kinase delta inhibitor is (S)-2-(1-(4-amino-3-(3-fluoro-4-isopropoxyphenyl)-1H-pyrazolo[3,4-d]pyrim-idin-1-yl)ethyl)-6-fluoro -3-(3-fluorophenyl)-4H-chromen-4-one, or a pharmaceutically acceptable salt, solvate, or prodrug thereof; and
(ii) about 900 mg of ublituximab once every 1 or 2 weeks; wherein the first dose of ublituximab is administered on day 1 of the induction phase;
wherein the duration of the induction phase is about 8 weeks; and
wherein the PI3-kinase delta inhibitor and the ublituximab are administered to the subject simultaneously, sequentially or both simultaneously and sequentially.

123. A PI3-kinase delta inhibitor and/or ublituximab for use according to claim 122, wherein the first dose of ublituximab is divided into 2 sub-doses during the induction phase, wherein the first sub-dose comprises up to 150 mg of ublituximab; and the second sub-dose comprises up to 750 mg of ublituximab; and wherein the first and second sub-doses are administered on day 1 and day 2 of the induction phase, respectively.

124. A PI3-kinase delta inhibitor and/or ublituximab for use according to any one of claims 121-123, further comprising, after the treatment phase, a maintenance phase, comprising administering to the subject daily about 800 mg of the PI3-kinase delta inhibitor, or a pharmaceutically acceptable salt, solvate or prodrug thereof, wherein the duration of the maintenance phase is at least 3 weeks.

125. A PI3-kinase delta inhibitor and/or ublituximab for use according to any one of claims 121-124, wherein the PI3-kinase delta inhibitor is micronized and is formulated for oral administration.

126. A PI3-kinase delta inhibitor and/or ublituximab for use according to any one of claims 121-125, wherein the ublituximab and the pembrolizumab are formulated for intravenous infusion.

127. A PI3-kinase delta inhibitor and/or ublituximab for use in a method of treating a subject afflicted with a hematologic cancer, comprising administering to the subject in a treatment phase:
(i) a therapeutically effective amount of the PI3 kinase-delta inhibitor;
(ii) a therapeutically effective amount of an anti-CD20 antibody; and
(iii) a therapeutically effective amount of an anti-PD-1 or anti-PD-L1 antibody.

128. A PI3-kinase delta inhibitor and/or ublituximab for use according to claim 127, wherein the PI3 kinase-delta inhibitor is (S)-2-(1-(4-amino-3-(3-fluoro-4-isopropoxyphenyl)-1H-pyrazolo [3,4-d]pyrim-idin-1-yl)ethyl-6-fluoro-3-(3-fluorophenyl)-4H-chromen-4-one, or a pharmaceutically acceptable salt, solvate, or prodrug thereof.

129. A PI3-kinase delta inhibitor and/or ublituximab for use according to claim 128, wherein the PI3 kinase-delta inhibitor is in the form of a p-toluenesulfonic acid (PTSA) salt.

130. A PI3-kinase delta inhibitor and/or ublituximab for use according to any one of claims 127-129, wherein the anti-CD20 antibody is ublituximab or an antibody fragment that binds the same epitope as ublituximab.

131. A PI3-kinase delta inhibitor and/or ublituximab for use according to any one of claims 127-130, wherein the anti-PD-1 antibody is nivolumab, pembrolizumab, or pidilizumab.

132. A PI3-kinase delta inhibitor and/or ublituximab for use according to any one of claims 127-130, wherein the anti-PD-L1 antibody is CTI-07, CTI-09, CTI-48, CTI-49, CTI-50, CTI-76, CTI-77, CTI-78, CTI-57, CTI-58, CTI-97, CTI-98, CTI-92, CTI-95, CTI-93, CTI-94, CTI-96, durvalumab, BMS-936559, atezolizumab, or avelumab.

133. A PI3-kinase delta inhibitor and/or ublituximab for use according to any one of claims 127-132, wherein the hematological cancer is lymphoma, leukemia, or myeloma.

134. A PI3-kinase delta inhibitor and/or ublituximab for use according to claim 133, wherein the hematological cancer is acute lymphocytic leukemia (ALL), acute myeloid leukemia (AML), chronic lymphocytic leukemia (CLL), small lymphocytic lymphoma (SLL), multiple myeloma (MM), non-Hodgkin's lymphoma (NHL), mantle cell lymphoma (MCL), follicular lymphoma (FL), Waldenstrom's macroglobulinemia (WM), diffuse large B-cell lymphoma (DLBCL), marginal zone lymphoma (MZL), hairy cell leukemia (HCL), Burkitt's lymphoma (BL), or Richter's transformation.

135. A PI3-kinase delta inhibitor and/or ublituximab for use according to claim 133 or 134, wherein the hematological cancer expresses PD-1 or PD-L1.

136. A PI3-kinase delta inhibitor and/or ublituximab for use according to any one of claims 133-135, wherein the hematological cancer is relapsed-refractory disease.

137. A PI3-kinase delta inhibitor and/or ublituximab for use according to any one of claims 133-136, wherein the hematological cancer is relapsed-refractory CLL.

138. A method of treating a subject afflicted with a hematologic cancer, comprising administering to the subject in a treatment phase:
(i) a therapeutically effective amount of the PI3 kinase-delta inhibitor, wherein said PI3 kinase-delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate, or prodrug thereof;
(ii) a therapeutically effective amount of ublituximab; and
(iii) a therapeutically effective amount of an anti-PD-1 antibody or anti-PD-L1 antibody.

139. A PI3-kinase delta inhibitor and/or ublituximab for use according to claim 138, wherein the anti-PD-1 antibody is nivolumab, pembrolizumab, or pidilizumab.

140. A PI3-kinase delta inhibitor and/or ublituximab for use according to claim 138, wherein the anti-PD-L1 antibody is CTI-07, CTI-09, CTI-48, CTI-49, CTI-50, CTI-76, CTI-77, CTI-78, CTI-57, CTI-58, CTI-97, CTI-98, CTI-92, CTI-95, CTI-93, CTI-94, CTI-96, durvalumab, BMS-936559, atezolizumab, or avelumab.

141. A PI3-kinase delta inhibitor and/or ublituximab for use according to any of claims 78-140, wherein the subject is a human.

142. A PI3-kinase delta inhibitor and/or ublituximab for use according to any of claims 127-141, further comprising, prior to the treatment phase, an induction phase, comprising administering to the subject:
(i) a therapeutically effective amount of the PI3 kinase-delta inhibitor, wherein said PI3 kinase-delta inhibitor is (S)-2-(1-(4-amino-3-(3-fluoro-4-isopropoxyphenyl)-1H-pyrazolo[3,4-d]pyrim-idin-1-yl)ethyl-6-fluoro-3-(3-fluorophenyl)-4H-chromen-4-one, or a pharmaceutically acceptable salt, solvate or prodrug thereof; and
(ii) a therapeutically effective amount of ublituximab.

143. A PI3-kinase delta inhibitor and/or ublituximab for use according to claim 142, wherein the PI3 kinase-delta inhibitor is in the form of a p-toluenesulfonic acid (PTSA) salt.

144. A PI3-kinase delta inhibitor and/or ublituximab for use according to any one of claims 127-143, further comprising, after the treatment phase, a maintenance phase, comprising administering to the subject a therapeutically effective amount of the PI3 kinase-delta inhibitor, or a pharmaceutically acceptable salt, solvate or prodrug thereof.

145. A PI3-kinase delta inhibitor and/or ublituximab for use according to any one of claims 127-144, wherein the PI3 kinase-delta inhibitor, the ublituximab, and the anti-PD-1 antibody or anti-PD-L1 antibody are administered to the subject simultaneously, sequentially, or both simultaneously and sequentially.

146. A PI3-kinase delta inhibitor and/or ublituximab for use according to any one of claims 78-145, wherein the PI3 kinase-delta inhibitor is TGR-1202 (umbralisib tosylate).

147. A PI3-kinase delta inhibitor and/or ublituximab for use according to any one of claim 127-130, 132-138, or 140-146, wherein the anti-PD-L1 antibody is CTI-48.
